SEQUENCE LISTING (19 sequences)

SEQ ID NO: 1 - protein, 508 aa, unidentified
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNKFNKDQQSAFYEILNMPNLNEEQRNGFIQSLKDDPSQSTNVLGEAKKLNESQAPKADNNFNKEQQNAFYEILNMPNLNEEQRNGFIQSLKDDPSQSANLLAEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDGNKPGKEDGNKPGKEDNKKPGKEDGNKPGKEDNKKPGKEDGNKPGKEDGNKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 2 - protein, 593 aa, unidentified
MEKEKKVKYFLRKSAFGLASVSAAFLVGSTVFAVDSPIEDTPIIRNGGELTNLLGNSETTLALRNEESATADLTAAAVADTVAAAAAENAGAAAWEAAAAADALAKAKADALKEFNKYGVSDYYKNLINNAKTVEGVKDLQAQVVESAKKARISEATDGLSDFLKSQTPAEDTVKSIELAEAKVLANRELDKYGVSDYHKNLINNAKTVEGVKDLQAQVVESAKKARISEATDGLSDFLKSQTPAEDTVKSIELAEAKVLANRELDKYGVSDYYKNLINNAKTVEGVKALIDEILAALPKTDTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEMVTEVPGDAPTEPEKPEASIPLVPLTPATPIAKDDAKKDDTKKEDAKKPEAKKEDAKKAETLPTTGEGSNPFFTAAALAVMAGAGALAVASKRKED

SEQ ID NO: 3 - protein, 992 aa, unidentified
MKINKKLLMAALAGAIVVGGGANAYAAEEDNTDNNLSMDEISDAYFDYHGDVSDSVDPVEEEIDEALAKALAEAKETAKKHIDSLNHLSETAKKLAKNDIDSATTINAINDIVARADVMERKTAEKEEAEKLAAAKETAKKHIDELKHLADKTKELAKRDIDSATTINAINDIVARADVMERKTAEKEEAEKLAAAKETAKKHIDELKHLADKTKELAKRDIDSATTIDAINDIVARADVMERKLSEKETPEPEEEVTIKANLIFADGSTQNAEFKGTFAKAVSDAYAYADALKKDNGEYTVDVADKGLTLNIKFAGKKEKPEEPKEEVTIKVNLIFADGKTQTAEFKGTFEEATAKAYAYADLLAKENGEYTADLEDGGNTINIKFAGKETPETPEEPKEEVTIKVNLIFADGKIQTAEFKGTFEEATAKAYAYANLLAKENGEYTADLEDGGNTINIKFAGKETPETPEEPKEEVTIKVNLIFADGKTQTAEFKGTFEEATAEAYRYADLLAKVNGEYTADLEDGGYTINIKFAGKEQPGENPGITIDEWLLKNAKEEAIKELKEAGITSDLYFSLINKAKTVEGVEALKNEILKAHAGEETPELKDGYATYEEAEAAAKEALKNDDVNNAYEIVQGADGRYYYVLKIEVADEEEPGEDTPEVQEGYATYEEAEAAAKEALKEDKVNNAYEVVQGADGRYYYVLKIEDKEDEQPGEEPGENPGITIDEWLLKNAKEDAIKELKEAGISSDIYFDAINKAKTVEGVEALKNEILKAHAEKPGENPGITIDEWLLKNAKEAAIKELKEAGITAEYLFNLINKAKTVEGVESLKNEILKAHAEKPGENPGITIDEWLLKNAKEDAIKELKEAGITSDIYFDAINKAKTIEGVEALKNEILKAHKKDEEPGKKPGEDKKPEDKKPGEDKKPEDKKPGEDKKPEDKKPGKTDKDSPNKKKKAKLPKAGSEAEILTLAAAALSTAAGAYVSLKKRK

SEQ ID NO: 4 - protein, 55 aa, unidentified
MKFNKEQQNAFYEILHLPNLNEEQRNGFIQSLKDDPSQSANLLAEAKKLNDAQAP

SEQ ID NO: 5 - protein, 55 aa, unidentified
MSFNMEQQNAFYEILHLPNLNEEQRNGFIQSLRDDPSQSANLLAEAQELNDAQAP

SEQ ID NO: 6 - protein, 57 aa, unidentified
MKACGNKEQQNAFYEILHLPNLNEEQRNGFIQSLKDDPSQSANLLAEAKKLNDAQAP

SEQ ID NO: 7 - protein, 57 aa, unidentified
MKACGNKEQQNAFYEILHLPNLNEEQRNGFIQSLKDDPCQSANLLAEAKKLNDAQAP

SEQ ID NO: 8 - protein, 57 aa, unidentified
MKACGNMEQQNAFYEILHLPNLNEEQRNGFIQSLRDDPSQSANLLAEAQELNDAQAP

SEQ ID NO: 9 - protein, 57 aa, unidentified
MKACGNMEQQNAFYEILHLPNLNEEQRNGFIQSLRDDPCQSANLLAEAQELNDAQAP

SEQ ID NO: 10 - protein, 61 aa, unidentified
MKGGGGASFCMEQQNAFYEILHLPNLNEEQRNGFIQSLRDCPSQSANLLAEAQELNDAQAP

SEQ ID NO: 11 - protein, 61 aa, unidentified
MKGGGGASFNMCQQNAFYEILHLPNLNEEQRNGFIQSLRDCPSQSANLLAEAQELNDAQAP

SEQ ID NO: 12 - protein, 57 aa, unidentified
MKTYKLILNGKTLKGETTTEAVDAATAEKVFKQYANDNGVDGEWTYDDATKTFTVTE

SEQ ID NO: 13 - protein, 57 aa, unidentified
MKTYKLVINGKTLKGETTTEAVDAATAEKVFKQYANDNGVDGEWTYDDATKTFTVTE

SEQ ID NO: 14 - protein, 57 aa, unidentified
MKTYKLVINGKTLKGETTTKAVDAETAEKAFKQYANDNGVDGVWTYDDATKTFTVTE

SEQ ID NO: 15 - protein, 57 aa, unidentified
MSTYRLILNGVTLSGETTTEAVDAATAERVFRQYANDYGVDGEWTYDDATRTFTVTE

SEQ ID NO: 16 - protein, 65 aa, unidentified
MKEEVTIKVNLIFADGKTQTAEFKGTFEEATAKAYAYADLLAKENGEYTADLEDGGNTINIKFAG

SEQ ID NO: 17 - protein, 65 aa, unidentified
MAEEVTIRVNLIFADGSTQTAEFRGTFEEATAEAYAYADLLARENGEYTADLEDGGNTINIRFAG

SEQ ID NO: 18 - protein, 76 aa, unidentified
MATKASKGGGGTTYRLILNGVTLSGETTTEACDAATAERVFRQYANDYGVDGEWTYDCATRTFTVTEGEDEDEDED

SEQ ID NO: 19 - protein, 84 aa, unidentified
MATKASKGGGGASEEVTIRVNLIFADGSTQCAEFRGTFEEATAEAYAYADLLARENGEYTACLEDGGNTINIRFAGEDEDEDED

(19) United States
(12) Patent Application Publication
(10) Pub. No.: US 2026/0265179 A1
(43) Pub. Date: Sep. 10, 2026

(54) PHOTOSWITCHABLE COMPOUNDS AND AFFINITY LIGANDS, AND THEIR USE FOR THE OPTICAL CONTROL OF AFFINITY MATRICES

(71) Applicant: AFC Innovations GmbH, Garching (DE)

(72) Inventors: Andreas Reichert, Freising (DE); Christopher Graf, Bodenwöhr (DE); Fabian Rodewald, Freising (DE); Ingmar Polte, München (DE)

(21) Appl. No.: 19/160,927

(22) PCT Filed: Mar. 1, 2024
(86) PCT No.: PCT/EP2024/055494

§ 371 Date: Aug. 29, 2025

(30) Foreign Application Priority Data
Mar. 1, 2023 (EP) 23159528.1

(51) Int. Cl. C07C 245/08; C07C 311/21; C07D 207/452; C07K 1/13; C07K 1/22; G01N 30/02; G01N 30/52

(52) U.S. Cl. CPC C07C 245/08; C07C 311/21; C07D 207/452; C07K 1/13; C07K 1/22; G01N 30/02; G01N 2030/027; G01N 2030/525

(57) ABSTRACT

The present disclosure relates to a photoswitchable azobiaryl compound, a photoswitchable affinity ligand, a photoswitchable affinity matrix, the use of a photoswitchable compound, a photoswitchable affinity ligand, or a photoswitchable affinity matrix for isolating and/or purifying a target molecule, a method of isolating and/or purifying a target molecule, and a process for the preparation of a photoswitchable azobiaryl compound.

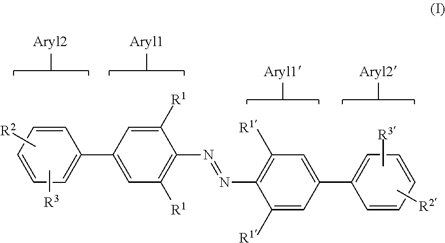

### TECHNICAL FIELD

The present disclosure relates to a photoswitchable azobiaryl compound, a photoswitchable affinity ligand, a photoswitchable affinity matrix, the use of a photoswitchable compound, a photoswitchable affinity ligand, or a photoswitchable affinity matrix for isolating and/or purifying a target molecule, a method of isolating and/or purifying a target molecule, and a process for the preparation of a photoswitchable azobiaryl compound.

### BACKGROUND OF THE INVENTION

Generally, chromatographic methods are used to separate and/or purify molecules, compounds or substances of interest such as proteins, nucleic acids, virus particles, cells, and polysaccharides from a composition of different substances. Affinity chromatography specifically involves passing such a composition over an affinity matrix comprising a ligand that is specific (i.e., a specific binding partner) for the target molecule comprised in the composition.

Upon contacting the ligand under conditions that allow strong binding, the target molecule is bound to the matrix and is therefore retained from the composition. After subsequent depletion of contaminating components, an elution buffer is commonly used to favor dissociation of the target molecule from the affinity matrix in the final step.

Affinity chromatography provides certain advantages over other types of chromatography. For example, affinity chromatography provides a purification method that can isolate a target protein from a mixture of the target protein and other biomolecules in a single step with high selectivity and high yield. Despite the advantages of current affinity chromatography methods, there exists a need to improve the mechanism of elution, often viewed as the most critical process step. The elution should ideally be carried out in a way that keeps the affinity matrix intact, allowing regeneration and multiple uses.

While binding of the target molecule to the affinity matrix occurs under mild buffer conditions that mimic the native environment regarding pH and ionic strength, the elution step often requires a drastic change, for example by strongly altering the pH, polarity, or ionic strength (Hage D S et al., J Pharm Biomed Anal. 2012 October; 69:93-105. doi: 10.1016/j.jpba.2012.01.004).

Unspecific elution conditions like altered pH, high concentrations of salts, organic cosolvents, detergents, metal ions, chelators, or reducing agents often impair the target molecule and/or the affinity matrix. Particularly if the target molecule is a protein, such elution conditions can result in denaturation, aggregation, or chemical modification, thus hampering the physiochemical properties or functional activity.

Alternatively, a competitor can be added to the elution buffer to displace the target molecule bound to the affinity ligand, for example imidazole in the case of the His₆-tag (Hochuli, E. et al., Nat Biotechnol 6, 1321-1325 (1988). https://doi.org/10.1038/nbt1188-1321). Evidently, using such small molecules as competing agents for elution results in contamination of the solution comprising the purified target molecule. Consequently, these reagents must be removed in time-consuming additional purification steps, for example by dialysis or gel filtration. Furthermore, after elution of the target molecule, the affinity matrix must be regenerated in a resource consuming procedure prior to the next round of sample application.

Perhaps the greatest success of affinity chromatography at scale has been achieved in the field of biopharmaceutical monoclonal antibody purification. In modern biotechnology and medicine, antibodies are an integral part of almost all fields of application. Especially for the treatment of life-threatening diseases, such as cancer or autoimmune disorders, antibodies have been a consistent game-changer.

However, antibody manufacturing is a challenging and very expensive process. This is reflected by high prices for antibody products and even higher costs for antibody therapeutics, limiting the impact on global health issues. These valuable proteins require engineering and manufacturing before they may be used. It is necessary for the researcher, or manufacturer, to isolate the antibodies from a crude extract.

A key step in the isolation of such antibodies is the well-established affinity chromatography with protein A resins. Protein A chromatography is a simple and highly selective method relying on the strong and specific interaction between protein A and the crystallizable fragment (Fc) of the antibody (Hober S et al., J Chromatogr B Analyt Technol Biomed Life Sci. 2007 Mar. 15; 848(1):40-7. doi: 10.1016/j.jchromb.2006.09.030).

However, the elution step poses a significant disadvantage for protein A purification because the target antibody is eluted from the column using a strong shift in buffer pH to acidic conditions. By this means, antibodies can be isolated to a high degree of purity but at the expense of non-optimal buffer conditions and adverse pH effects on this type of molecule. These conditions might even exclude sensitive variants or conjugates entirely from purification.

Alternatives to protein A have been explored, such as cation exchange, and non-chromatographic methods like precipitation, but they have not been able to compete with the well-established protein A platform when applied to industrial-scale bioprocessing (Kelley B. MAbs. 2009 September-October; 1(5):443-52. doi: 10.4161/mabs.1.5.9448).

Thus, there is a need in the art for improved affinity matrices, which are suitable for fast and simple isolation and/or purification of a target molecule. Further, there is a need for improved affinity matrices, wherein contamination and biochemical modification or aggregation of the eluted target molecule are reduced. Further, there is a need in the art for improved affinity matrices which meet the demand of modern chromatography processes in terms of short residence times, while providing the same binding capacity and reusability.

### SUMMARY OF THE INVENTION

These and other technical problems are solved by provision of the embodiments as defined herein and as characterized in the claims.

According to the first aspect of the present invention, a photoswitchable azobiaryl compound is provided according to formula (I)

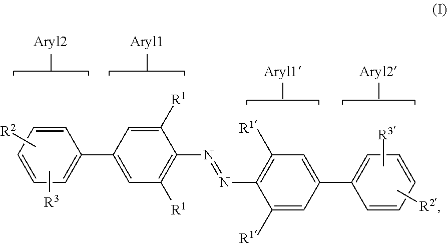

wherein R¹ and R^{1′} are independently selected from the group comprising H, F, Cl, Br, (CH₂)ₙCH₃, CH((CH₂)ₙCH₃)((CH₂)ₓCH₃), C((CH₂)ₙCH₃)((CH₂)ₓCH₃)((CH₂)_{z}CH₃), (CH₂)ₙCH((CH₂)ₓCH₃)((CH₂)_{z}CH₃), (CH₂)ₙC((CH₂)ₓCH₃)((CH₂)_{y}CH₃)((CH₂)_{z}CH₃), O(CH₂)ₙCH₃, OCH((CH₂)ₙCH₃)((CH₂)ₓCH₃), OC((CH₂)ₙCH₃)—((CH₂)ₓCH₃)((CH₂)_{z}CH₃), O(CH₂)ₙCH((CH₂)ₓCH₃)((CH₂)_{z}CH₃), O(CH₂)ₙC((CH₂)ₓCH₃)—((CH₂)_{y}CH₃)((CH₂)_{z}CH₃), S(CH₂)ₙCH₃, SCH((CH₂)ₙCH₃)((CH₂)ₓCH₃), SC((CH₂)ₙCH₃)((CH₂)ₓCH₃)—((CH₂)_{z}CH₃), S(CH₂)ₙCH((CH₂)ₓCH₃)((CH₂)_{z}CH₃), S(CH₂)ₙC((CH₂)ₓCH₃)((CH₂)_{y}CH₃)((CH₂)_{z}CH₃), NH₂, NH(CH₂)ₙCH₃, NHCH((CH₂)ₙCH₃)—((CH₂)ₓCH₃), NHC((CH₂)ₙCH₃)((CH₂)ₓCH₃)((CH₂)_{z}CH₃), NH(CH₂)ₙCH((CH₂)ₓCH₃)((CH₂)_{z}CH₃), NH(CH₂)ₙC—((CH₂)ₓCH₃)—((CH₂)_{y}CH₃)((CH₂)_{z}CH₃), N((CH₂)ₙCH₃)₂, N(CH((CH₂)ₙCH₃)((CH₂)ₓCH₃))₂, N(C((CH₂)ₙCH₃)—((CH₂)ₓCH₃)((CH₂)_{z}CH₃))₂, N((CH₂)ₙCH((CH₂)ₓCH₃)((CH₂)_{z}CH₃))₂, N((CH₂)ₙC((CH₂)ₓCH₃)((CH₂)_{y}CH₃)—((CH₂)_{z}CH₃))₂, aziridin-1-yl, azetidin-1-yl, pyrrolidin-1-yl, piperidin-1-yl, azepan-1-yl, azocan-1yl, morpholin-1-yl, 4-methyl-piperazin-1-yl, wherein n may be any integer from 0 to 10, wherein x may be any integer from 0 to 10, wherein y may be any integer from 0 to 10 and wherein z may be any integer from 0 to 10;
R² and R^{2′} are independently selected from the group comprising SO₃H, SO₃Li, SO₃Na, SO₃K, COOH, COONHS, CONH₂, CONH—(CH₂)ₙCH₃, CONH—((CH₂)ₙCH₃)((CH₂)_{z}CH₃), CONH—(CH₂)ₙSO₃H, CONH—(CH₂)ₙSO₃Li, CONH—(CH₂)ₙSO₃Na, CONH—(CH₂)ₙSO₃K, CONH—(CH₂)ₙCCH, CONH—(CH₂)ₙN₃, CONH—(CH₂)ₙNH₂, CONH—(CH₂)ₙCOOH, CONH—(CH₂)ₙNHAcI, CONH—(CH₂)ₙNHAcBr, CONH—(CH₂)ₙNHAcCl, CONH—(CH₂)ₙN(maleimide), CONH—(CH₂)ₙ—NH(2-chloromethyl acrylate), CONH—(CH₂)ₙ—NH(vinylsulfonate), CONH—(CH₂)ₙ—NHCOPhF₅, CONH—(CH₂)ₙ—NHSO₂PhF₅, CONH—(CH₂)ₙCOONHS, CONH-PEG-OH, CONH-PEG-NH₂, CONH-PEG-COOH, CONH-PEG-COONHS, CONH-PEG-O(CH₂)ₙSO₃H, CONH-PEG-O(CH₂)ₙSO₃Li, CONH-PEG-O(CH₂)ₙSO₃Na, CONH-PEG-O(CH₂)ₙSO₃K, CONH-PEG-NHAcI, CONH-PEG-NHAcBr, CONH-PEG-NHAcCl, CONH-PEG-N(maleimide), CONH—PEG-NH(2-chloromethyl acrylate), CONH-PEG-NH(vinylsulfonate), CONH-PEG-NHCOPhF₅, CONH—PEG-NHSO₂PhF₅, CONH-PEG-N₃, CONH-PEG-OCH₂CCH, CONH-PEG-NHCH₂CCH, CONH-PEG-N(CH₂CCH)₂, CONH-PEG-NHCO(CH₂)ₙCOOH, CONH-PEG-NHCO(CH₂)ₙCOONHS, CONH—(Xaa)ₙ-OH, CONH—(Xaa)ₙ-NH(CH₂)ₙSO₃H, CONH—(Xaa)ₙ-NH(CH₂)ₙSO₃Li, CONH—(Xaa)ₙ-NH(CH₂)ₙSO₃Na, CONH—(Xaa)ₙ-NH(CH₂)ₙSO₃K, CONH—(Xaa)ₙ-OMe, CONH—(Xaa)ₙ-ONHS, CONH—(Xaa)ₙ-ONHS, CONH—(Xaa)ₙ-NH(CH₂)ₙCCH, CONH—(Xaa)ₙ-NH(CH₂)ₙN₃, CONH—(Xaa)ₙ-NH(CH₂) zNHAcl, CONH—(Xaa)ₙ-NH(CH₂)_{z}NHAcBr, CONH—(Xaa)ₙ-NH(CH₂)_{z}NHAcCI, CONH—(Xaa)ₙ-NH(CH₂)_{z}-N(maleimide), CONH—(Xaa)ₙ-NH(CH₂)_{z}NH(2-chloromethyl acrylate), CONH—(Xaa)ₙ-NH(CH₂)_{z}NH(vinylsulfonate), CONH—(Xaa)ₙ-NH(CH₂)_{z}NHCOPhF₅, CONH—(Xaa)ₙ-NH(CH₂)_{z}NHSO₂PhF₅, CONH—(Xaa)ₙ-N₃, CONH—(Xaa)ₙ-OCH₂CCH, CONH—(Xaa)ₙ-NHCH₂CCH, CONH—(Xaa)ₙ-N(OCH₂CCH)₂, CONH—(Xaa)ₙ-NH—(CH₂)ₙCOOH, CONH—(Xaa)ₙ-NH—(CH₂)ₙCOONHS, CONH—(Xaa)ₙ-NH-PEG-OH, CONH—(Xaa)ₙ-NH-PEG-NH₂, CONH—(Xaa)ₙ-NH-PEG-COOH, CONH—(Xaa)ₙ-NH-PEG-COONHS, CONH—(Xaa)ₙ-NH-PEG-NHAcl, CONH—(Xaa)ₙ-NH-PEG-NHAcBr, CONH—(Xaa)ₙ-NH-PEG-NHACCI, CONH—(Xaa)ₙ-NH-PEG-N(maleimide), CONH—(Xaa)ₙ-NH-PEG-NH(2-chloromethyl acrylate), CONH—(Xaa)ₙ-NH-PEG-NH(vinylsulfonate), CONH—(Xaa)ₙ-NH-PEG-NHCOPhF₅, CONH—(Xaa)ₙ-NH-PEG-NHSO₂PhF₅, CONH—(Xaa)ₙ-NH-PEG-N₃, CONH—(Xaa)ₙ-NH-PEG-OCH₂CCH, CONH—(Xaa)ₙ-NH-PEG-NHCH₂CCH, CONH—(Xaa)ₙ-NH-PEG-N(CH₂CCH)₂, CONH—(Xaa)ₙ-NH-PEG-NHCO(CH₂)ₙCOOH, CONH—(Xaa)ₙ-NH-PEG-NHCO(CH₂)ₙCOONHS, wherein Xaa can be any canonical or non-canonical amino acid, wherein n may be any integer from 0 to 10, and wherein z may be any integer from 0 to 10;
R³ and R^{3′} are independently selected from the group comprising H, NH₂, NHCO (C₁-C₆-alkyl), NHCO (C₁-C₆-haloalkyl), NHBoc, NHCbz, NHalloc, NH(CH₂)ₙCH₃, NHCH((CH₂)ₙCH₃)((CH₂)ₓCH₃), NHC((CH₂)ₙCH₃)((CH₂)ₓCH₃)((CH₂)_{z}CH₃), NH(CH₂)ₙCH((CH₂)ₓCH₃)((CH₂)_{z}CH₃), NH(CH₂)ₙC((CH₂)ₓCH₃)—((CH₂)_{y}CH₃)((CH₂)_{z}CH₃), N((CH₂)ₙCH₃)₂, N(CH((CH₂)ₙCH₃)—((CH₂)ₓCH₃))₂, N(C((CH₂)ₙCH₃)((CH₂)ₓCH₃)((CH₂)_{z}CH₃))₂, N((CH₂)ₙCH((CH₂)ₓCH₃)—((CH₂)_{z}CH₃))₂, N((CH₂)ₙC((CH₂)ₓCH₃)((CH₂)_{y}CH₃)((CH₂)_{z}CH₃))₂, aziridin-1-yl, azetidin-1-yl, pyrrolidin-1-yl, piperidin-1-yl, azepan-1-yl, azocan-1yl, morpholin-1-yl, 4-methyl-piperazin-1-yl, NHCH₂CHCH₂, N(CH₂CHCH₂)₂, NHCH₂CCH, N(CH₂CCH)₂, NHCOCCH, NHCO(CH₂)ₙN₃, NHacrylate, NH(2-chloromethyl acrylate), NH(vinyl sulfonate), N(maleimide), N(2-bromomaleimide), N(2,3-dibromomaleimide), NHCOaryl, NHCOhaloaryl, NHSO₂aryl, NHSO₂haloaryl, (CH₂)ₙN(maleimide), (CH₂)ₙN(2-bromomaleimide), (CH₂)ₙN(2,3-dibromomaleimide), NHCO(CH₂)ₙN(maleimide), NHCO(CH₂)ₙN(2-bromomaleimide), NHCO(CH₂)ₙN(2,3-dibromomaleimide), NCS, NCO, NH(CH₂)ₙCH(O)CH₂, N((CH₂)ₙCH(O)CH₂)₂, NACCH₂CH(O)CH₂ wherein n may be any integer from 0 to 10, and wherein z may be any integer from 0 to 10.

The person skilled in the art will be aware that if the moiety ends on “NHS”, the term denotes “N-succinimidyl”, which is derived from N-Hydroxysuccinimide so that e.g. COONHS refers to carboxy N-succinimidyl ester and (Xaa)n-ONHS refers to C-terminal N-succinimidyl ester of peptide or amino acid.

Further, it is to be understood that “CCH” denotes an alkyne moiety, i.e. a triple bond, so that e.g. CONH—(CH₂)ₙCCH refers to CONH—(CH₂)ₙethinyl.

In a preferred embodiment, at least one of R¹ or R^{1′} is not H.

In a preferred embodiment of the first aspect of the invention, Aryl¹-Aryl² and Aryl¹′-Aryl^{2′} are multifunctionalized biaryl moieties, more preferably wherein at least one R¹ and at least one R^{1′} is not hydrogen, even more preferably wherein both R¹ and both R^{1′} are not hydrogen.

It is to be understood that the term “multifunctionalized biaryl moieties” denotes that the respective moiety possesses more than one substitute. Thus, if Aryl¹-Aryl² is a multifunctionalized biaryl moiety, the two linked aryls comprise at least two substituents.

In another preferred embodiment of the first aspect of the invention, at least one, preferably both, of R² and R^{2′} is CONH-Xaa-OH (Xaa=any canonical or non-canonical amino acid), or at least one, preferably both, of R² and R^{2′} comprise a linker selected from the group consisting of peptides, bifunctional alkanes, poly(alkylene oxides), more preferably wherein said poly(alkylene oxides) has a molecular weight selected from the group consisting of between about 100 g/mol and about 80,000 g/mol, even more preferably between about 100 g/mol and 6,000 g/mol. In another preferred embodiment of the first aspect of the invention, at least one of R² or R^{2′} is not COOH, preferably wherein both R² and R^{2′} are not COOH.

The terms “about” in the context of the present invention denotes an interval of accuracy that a person skilled in the art will understand to still ensure the technical effect of the feature in question. The term typically indicates a deviation from the indicated numerical value of ±5%, preferably ±2%, more preferably ±1%.

In one preferred embodiment of the first aspect of the invention, at least one, preferably both, of R³ and R^{3′} is a substituent selected from the group comprising amines, acrylamides, NHCO (C₁-C₆-haloalkyl) such as α-haloacetamides, vinyl sulfonates, isothiocyanates, isocyanates, epoxides, maleimides, haloaryl carboxy- and haloaryl sulfonamides such as fluorophenyl carboxy- and fluorophenyl sulfonamides, more preferably wherein R³ and R^{3′} are able to form an intramolecular linkage within an affinity ligand via amino acid side chains, even more preferably comprising a cysteine residue, a histidine residue, a lysine residue, a methionine residue or any canonical or non-canonical amino acid in the polypeptide that is accessible for reacting with the ligand-reactive moiety of the photoswitchable azobiaryl compound, alternatively more preferably wherein R³ and R^{3′} are able to form an intermolecular linkage between an affinity ligand and a second affinity ligand and/or a solid support via amino acid side chains, preferably involving a cysteine residue, a histidine residue, a lysine residue, a methionine residue or any canonical or non-canonical amino acid in the polypeptide or a functionality of the solid support that is accessible for reacting with the ligand-reactive moiety of the photoswitchable azobiaryl compound.

In another preferred embodiment of the first aspect of the invention, the compound is 4′-(2-(4-(2-iodoacetamido)-3′,5′-dimethoxybiphenyl-3-ylcarboxyamido)acetic acid)-4′-(2-(4-(2-iodoacetamido)-3′,5′-dimethoxybiphenyl-3-ylcarboxyamido)acetic acid)diazene.

In another preferred embodiment of the first aspect of the invention, the compound is 4′-(2-(4-(2-bromoacetamido)-3′, 5′-dimethoxybiphenyl-3-ylcarboxyamido)acetic acid)-4′-(2-(4-(2-bromoacetamido)-3′,5′-dimethoxybiphenyl-3-ylcarboxyamido)acetic acid)diazene.

In another preferred embodiment of the first aspect of the invention, the compound is 4′-(2-(4-(2-chloroacetamido)-3′,5′-dimethoxybiphenyl-3-ylcarboxyamido)acetic acid)-4′-(2-(4-(2-chloroacetamido)-3′,5′-dimethoxybiphenyl-3-ylcarboxyamido)acetic acid)diazene.

In another preferred embodiment of the first aspect of the invention, the compound is 4′-(2-(4-(2,5-dioxo-2,5-dihydro-1H-pyrrol-1-yl)acetamido)-3′,5′-dimethoxybiphenyl-3-ylcarboxamido)acetic acid)-4′-(2-(4-(2,5-dioxo-2,5-dihydro-1H-pyrrol-1-yl)acetamido)-3′,5′-dimethoxybiphenyl-3-ylcarboxamido)acetic acid)diazene. In another preferred embodiment of the first aspect of the invention, the compound is 4′-(2-(4-(perfluorophenyl)sulfonamido)-3′,5′-dimethoxybiphenyl-3-ylcarboxamido)acetic acid)-4′-(2-(4-(perfluorophenyl)sulfonamido)-3′,5′-dimethoxybiphenyl-3-ylcarboxamido)acetic acid)diazene. In another preferred embodiment of the first aspect of the invention, the compound is 4′-(2-(4-(perfluorobenzamido)-3′,5′-dimethoxybiphenyl-3-ylcarboxamido)acetic acid)-4′-(2-(4-(perfluorobenzamido))-3′,5′-dimethoxybiphenyl-3-ylcarboxamido)acetic acid)diazene. In another preferred embodiment of the first aspect of the invention, the compound is 4′-(2-(4-(2-iodoacetamido)-3′,5′-diethoxybiphenyl-3-ylcarboxyamido)acetic acid)-4′-(2-(4-(2-iodoacetamido)-3′,5′-diethoxybiphenyl-3-ylcarboxyamido)acetic acid)diazene. In another preferred embodiment of the first aspect of the invention, the compound is 4′-(2-(4-(2-bromoacetamido)-3′,5′-diethoxybiphenyl-3-ylcarboxyamido)acetic acid)-4′-(2-(4-(2-bromoacetamido)-3′,5′-diethoxybiphenyl-3-ylcarboxyamido) acetic acid)diazene. In another preferred embodiment of the first aspect of the invention, the compound is 4′-(2-(4-(2-chloroacetamido)-3′,5′-diethoxybiphenyl-3-ylcarboxyamido)acetic acid)-4′-(2-(4-(2-chloroacetamido)-3′,5′-diethoxybiphenyl-3-ylcarboxyamido)acetic acid)diazene. In another preferred embodiment of the first aspect of the invention, the compound is 4′-(2-(4-(2,5-dioxo-2,5-dihydro-1H-pyrrol-1-yl)acetamido)-3′,5′-diethoxybiphenyl-3-ylcarboxamido)acetic acid)-4′-(2-(4-(2,5-dioxo-2,5-dihydro-1H-pyrrol-1-yl)acetamido)-3′,5′-diethoxybiphenyl-3-ylcarboxamido)acetic acid)diazene. In another preferred embodiment of the first aspect of the invention, the compound is 4′-(2-(4-(perfluorophenyl)sulfonamido)-3′,5′-diethoxybiphenyl-3-ylcarboxamido)acetic acid)-4′-(2-(4-(perfluorophenyl) sulfonamido)-3′,5′-diethoxybiphenyl-3-ylcarboxamido)acetic acid)diazene. In another preferred embodiment of the first aspect of the invention, the compound is 4′-(2-(4-(perfluorobenzamido)-3′,5′-diethoxybiphenyl-3-ylcarboxamido)acetic acid)-4′-(2-(4-(perfluorobenzamido))-3′,5′-diethoxybiphenyl-3-ylcarboxamido)acetic acid)diazene. In another preferred embodiment of the first aspect of the invention, the compound is 4′-(2-(4-(2-iodoacetamido)-3′,5′-difluorobiphenyl-3-ylcarboxyamido)acetic acid)-4′-(2-(4-(2-iodoacetamido)-3′,5′-difluorobiphenyl-3-ylcarboxyamido)acetic acid)diazene. In another preferred embodiment of the first aspect of the invention, the compound is 4′-(2-(4-(2-bromoacetamido)-3′,5′-difluorobiphenyl-3-ylcarboxyamido)acetic acid)-4′-(2-(4-(2-bromoacetamido)-3′,5′-difluorobiphenyl-3-ylcarboxyamido)acetic acid)diazene. In another preferred embodiment of the first aspect of the invention, the compound is 4′-(2-(4-(2-chloroacetamido)-3′,5′-difluorobiphenyl-3-ylcarboxyamido)acetic acid)-4′-(2-(4-(2-chloroacetamido)-3′,5′-difluorobiphenyl-3-ylcarboxyamido)acetic acid)diazene. In another preferred embodiment of the first aspect of the invention, the compound is 4′-(2-(4-(2,5-dioxo-2,5-dihydro-1H-pyrrol-1-yl)acetamido)-3′,5′-difluorobiphenyl-3-ylcarboxamido)acetic acid)-4′-(2-(4-(2,5-dioxo-2,5-dihydro-1H-pyrrol-1-yl)acetamido)-3′,5′-difluorobiphenyl-3-ylcarboxamido)acetic acid)diazene. In another preferred embodiment of the first aspect of the invention, the compound is 4′-(2-(4-(perfluorophenyl)sulfonamido)-3′,5′-difluorobiphenyl-3-ylcarboxamido)acetic acid)-4′-(2-(4-(perfluorophenyl)sulfonamido)-3′,5′-difluorobiphenyl-3-ylcarboxamido)acetic acid)diazene. In another preferred embodiment of the first aspect of the invention, the compound is 4′-(2-(4-(perfluorobenzamido)-3′,5′-difluorobiphenyl-3-ylcarboxamido)acetic acid)-4′-(2-(4-(perfluorobenzamido))-3′,5′-difluorobiphenyl-3-ylcarboxamido)acetic acid)diazene. In another preferred embodiment of the first aspect of the invention, the compound is 4′-(2-(4-(2-iodoacetamido)-3′,5′-dichlorobiphenyl-3-ylcarboxyamido)acetic acid)-4′-(2-(4-(2-iodoacetamido)-3′,5′-dichlorobiphenyl-3-ylcarboxyamido)acetic acid)diazene. In another preferred embodiment of the first aspect of the invention, the compound is 4′-(2-(4-(2-bromoacetamido)-3′,5′-dichlorobiphenyl-3-ylcarboxyamido)acetic acid)-4′-(2-(4-(2-bromoacetamido)-3′,5′-dichlorobiphenyl-3-ylcarboxyamido)acetic acid)diazene. In another preferred embodiment of the first aspect of the invention, the compound is 4′-(2-(4-(2-chloroacetamido)-3′,5′-dichlorobiphenyl-3-ylcarboxyamido)acetic acid)-4′-(2-(4-(2-chloroacetamido)-3′,5′-dichlorobiphenyl-3-ylcarboxyamido) acetic acid)diazene. In another preferred embodiment of the first aspect of the invention, the compound is 4′-(2-(4-(2,5-dioxo-2,5-dihydro-1H-pyrrol-1-yl)acetamido)-3′,5′-dichlorobiphenyl-3-ylcarboxamido)acetic acid)-4′-(2-(4-(2,5-dioxo-2,5-dihydro-1H-pyrrol-1-yl)acetamido)-3′,5′-dichlorobiphenyl-3-ylcarboxamido)acetic acid)diazene. In another preferred embodiment of the first aspect of the invention, the compound is 4′-(2-(4-(perfluorophenyl)sulfonamido)-3′,5′-dichlorobiphenyl-3-ylcarboxamido)acetic acid)-4′-(2-(4-(perfluorophenyl)sulfonamido)-3′,5′-dichlorobiphenyl-3-ylcarboxamido)acetic acid)diazene. In another preferred embodiment of the first aspect of the invention, the compound is 4′-(2-(4-(perfluorobenzamido)-3′,5′-dichlorobiphenyl-3-ylcarboxamido)acetic acid)-4′-(2-(4-(perfluorobenzamido))-3′,5′-dichlorobiphenyl-3-ylcarboxamido)acetic acid)diazene. In another preferred embodiment of the first aspect of the invention, the compound is 4′-(2-(4-(2-iodoacetamido)-3′, 5′-dibromobiphenyl-3-ylcarboxyamido)acetic acid)-4′-(2-(4-(2-iodoacetamido)-3′,5′-dibromobiphenyl-3-ylcarboxyamido)acetic acid)diazene. In another preferred embodiment of the first aspect of the invention, the compound is 4′-(2-(4-(2-bromoacetamido)-3′,5′-dibromobiphenyl-3-ylcarboxyamido)acetic acid)-4′-(2-(4-(2-bromoacetamido)-3′,5′-dibromobiphenyl-3-ylcarboxyamido)acetic acid)diazene. In another preferred embodiment of the first aspect of the invention, the compound is 4′-(2-(4-(2-chloroacetamido)-3′,5′-dibromobiphenyl-3-ylcarboxyamido)acetic acid)-4′-(2-(4-(2-chloroacetamido)-3′,5′-dibromobiphenyl-3-ylcarboxyamido)acetic acid)diazene. In another preferred embodiment of the first aspect of the invention, the compound is 4′-(2-(4-(2,5-dioxo-2,5-dihydro-1H-pyrrol-1-yl)acetamido)-3′,5′-dibromobiphenyl-3-ylcarboxamido)acetic acid)-4′-(2-(4-(2,5-dioxo-2,5-dihydro-1H-pyrrol-1-yl)acetamido)-3′,5′-dibromobiphenyl-3-ylcarboxamido)acetic acid)diazene. In another preferred embodiment of the first aspect of the invention, the compound is 4′-(2-(4-(perfluorophenyl)sulfonamido)-3′,5′-dibromobiphenyl-3-ylcarboxamido)acetic acid)-4′-(2-(4-(perfluorophenyl)sulfonamido)-3′,5′-dibromobiphenyl-3-ylcarboxamido)acetic acid)diazene. In another preferred embodiment of the first aspect of the invention, the compound is 4′-(2-(4-(perfluorobenzamido)-3′,5′-dibromobiphenyl-3-ylcarboxamido)acetic acid)-4′-(2-(4-(perfluorobenzamido))-3′,5′-dibromobiphenyl-3-ylcarboxamido)acetic acid)diazene.

In a preferred embodiment of the first aspect of the invention, the compound is more compact in the cis-state and more stretched in the trans-state, more preferably wherein the distance between the flanking C⁴-atoms of the azobiaryl differs by about 0.5 nm to about 50 nm between cis and trans, even more preferably about 1 nm to about 30 nm, most preferably about 1 nm to about 20 nm.

In one preferred embodiment of the first aspect of the invention, the configuration of the photoswitchable azobiaryl compound can be altered by irradiation with (a) particular wavelength(s) of light in a reversible manner, more preferably wherein the configuration is switched from a cis to a trans-state, or alternatively more preferably wherein the configuration is switched from a trans to a cis-state.

In one preferred embodiment of the preceding embodiment of the first aspect of the invention, the wavelength(s) of light are at least 400 nm, more preferably at most 750 nm, even more preferably at most 700 nm.

In a preferred embodiment of the first aspect of the invention, at least 80% of the compound is in the trans-state when exposed to wavelength(s) of light from about 400 nm to 490 nm, and at least 80% is in the cis-state when exposed to wavelength(s) of light from about 600 nm to 700 nm.

In another preferred embodiment of the first aspect of the invention, the compound is soluble at pH 8.0 in water from about 0.001 mM to about 2 mM, more preferably more than 0.1 mM, most preferably more than 1 mM.

In a preferred embodiment of the first aspect of the invention, the thermal half-life of the cis-state at room temperature in water at pH 8.0 is from about 1 minute to about 72 h, more preferably more than 1 h, most preferably more than 12 h.

According to the second aspect of the present invention, a photoswitchable affinity ligand is provided comprising an affinity ligand in stable association with the photoswitchable azobiaryl compound according to the first aspect of the invention.

In a preferred embodiment of the second aspect of the invention, the affinity ligand is selected from the group consisting of a peptide, an oligopeptide, a polypeptide, a protein, an antibody or an antigen-binding fragment thereof, an immunoglobulin or a fragment thereof, an enzyme, a hormone, a cytokine, a complex, an oligonucleotide, a polynucleotide, a nucleic acid, a carbohydrate, a liposome, a nanoparticle, a cell, a biomacromolecule, a biomolecule or a small molecule.

In one preferred embodiment of the second aspect of the invention, the photoswitchable azobiaryl compound is stably associated with two conjugation sites within the affinity ligand in a bifunctional manner.

In another preferred embodiment of the second aspect of the invention, exposure to light of a specific wavelength induces a conformational switch causing a loss of specific affinity of the photoswitchable affinity ligand to the target molecule.

In a preferred embodiment of the second aspect of the invention, the affinity ligand is selected from the group comprising immunoglobulin (Ig)-binding proteins, more preferably selected from the group comprising protein A, protein G and protein L or variants thereof with the ability to specifically bind to immunoglobulins.

In a preferred embodiment of the second aspect of the invention, the affinity ligand has none, only one, or a defined set of lysine residues for site-directed immobilization to a solid phase.

In one preferred embodiment of the second aspect of the invention, the affinity ligand comprises the B domain of protein A (SEQ ID NO. 4), optionally carrying two substitutions of wild-type residues with cysteines, or comprises a protein domain having at least 80% sequence identity to SEQ ID NO. 4, more preferably wherein the affinity ligand comprises a protein domain having the amino acid sequence of SEQ ID NO. 6 or SEQ ID NO. 7, even more preferably wherein the affinity ligand comprises a protein domain having the amino acid sequence of SEQ ID NO. 7.

According to a preferred embodiment of the present invention, the dissociation constants (KD) for binding of affinity ligands as envisaged herein to the target molecule ranges between sub-nM to mM, and is preferably at most 1000 nM, more preferably at most 100 nM.

In another preferred embodiment of the second aspect of the invention, the affinity ligand comprises a lysine-deficient B domain of protein A (SEQ ID NO. 5), optionally substituted with two cysteine residues, or comprises a protein domain having at least 80% sequence identity to SEQ ID NO. 5, more preferably wherein the affinity ligand comprises a protein domain having the amino acid sequence of SEQ ID NO. 8, SEQ ID NO. 9, SEQ ID NO. 10 or SEQ ID NO. 11, even more preferably wherein the affinity ligand comprises a protein domain having the amino acid sequence of SEQ ID NO. 11.

In one preferred embodiment of the second aspect of the invention, the affinity ligand comprises at least one of the three homologous domains of protein G, defined as C1 (SEQ ID NO. 12), C2 (SEQ ID NO. 13) and C3 (SEQ ID NO. 14), optionally substituted with two cysteine residues, or comprises a protein domain having at least 80% sequence identity to SEQ ID NO. 12, SEQ ID NO. 13 or SEQ ID NO. 14. Preferably, the affinity ligand comprises C1, optionally substituted with two cysteine residues.

In another preferred embodiment of the second aspect of the invention, the affinity ligand comprises at least a lysine-deficient C1 domain of protein G (SEQ ID NO. 15), optionally substituted with two cysteine residues, or comprises a protein domain having at least 80% sequence identity to SEQ ID NO. 15.

In yet another preferred embodiment of the second aspect of the invention, the affinity ligand comprises at least a variant of the lysine-deficient C1 domain of protein G (SEQ ID NO. 18), or comprises a protein domain having at least 80% sequence identity to SEQ ID NO. 18.

Within the disclosure of the present invention, lysine-deficient shall indicate that a lysine present in a given amino acid sequence is exchanged for any other amino acid.

In one preferred embodiment of the second aspect of the invention, the affinity ligand comprises a C-domain of protein L, optionally substituted with two cysteine residues, or a protein domain having at least 80% sequence identity thereto, more preferably wherein the C-domain of protein L is the C2 domain of protein L (SEQ ID NO. 16) or a protein domain having at least 80% sequence identity thereto.

In another preferred embodiment of the second aspect of the invention, the affinity ligand comprises a lysine-deficient C2-domain of protein L (SEQ ID NO. 17), optionally substituted with two cysteine residues, or a protein domain having at least 80% sequence identity thereto. In yet another preferred embodiment of the second aspect of the invention, the affinity ligand comprises a variant of the lysine-deficient C2-domain of protein L (SEQ ID NO. 19) or a protein domain having at least 80% sequence identity thereto.

According to the third aspect of the present invention, a photoswitchable affinity matrix is provided comprising a solid support, and a photoswitchable azobiaryl compound according to the first aspect of the invention in stable association with an affinity ligand, wherein the photoswitchable azobiaryl compound and the affinity ligand are in stable association with the solid support, or a photoswitchable affinity ligand according to the second aspect of the invention in stable association with the solid support.

In another preferred embodiment of the third aspect of the invention, a photoswitchable affinity ligand is in stable association with the solid support via a site-specific attachment.

According to the fourth aspect of the present invention, the use of a photoswitchable compound is provided for isolating and/or purifying a target molecule.

In a preferred embodiment of the invention, in particular of the fourth aspect of the invention, the compound is not 4′-carboxyphenylazophenylalanine, more preferably the compound is not 3′-carboxyphenylazophenylalanine or 4′-carboxyphenylazophenylalanine, even more preferably the compound is not 3′-carboxyphenylazophenylalanine or a derivative thereof, and not 4′-carboxyphenylazophenylalanine or a derivative thereof.

In a preferred embodiment of the fourth aspect of the invention, the photoswitchable compound is a photoswitchable azobiaryl compound according to the first aspect of the invention.

According to the fifth aspect of the present invention, the use of a photoswitchable affinity ligand is provided comprising an affinity ligand in stable association with a photoswitchable compound for isolating and/or purifying a target molecule.

In a preferred embodiment of the fifth aspect of the invention, the photoswitchable compound is not 4′-carboxyphenylazophenylalanine, more preferably the compound is not 3′-carboxyphenylazophenylalanine or 4′-carboxyphenylazophenylalanine, even more preferably the compound is not 3′-carboxyphenylazophenylalanine or a derivative thereof, and not 4′-carboxyphenylazophenylalanine or a derivative thereof.

In a preferred embodiment of the fifth aspect of the invention, the photoswitchable affinity ligand is a photoswitchable affinity ligand according to the first aspect of the invention.

According to the sixth aspect of the present invention, the use of a photoswitchable affinity matrix according to the third aspect of the invention is provided for isolating and/or purifying a target molecule.

In a preferred embodiment of the fourth, fifth or sixth aspect of the invention, the target molecule is an immunoglobulin, more preferably the target molecule is an Fc-domain containing immunoglobulin, even more preferably an IgG type immunoglobulin.

According to the seventh aspect of the present invention, a method for isolating and/or purifying a target molecule is provided, wherein the method comprises the steps of providing a composition comprising a target molecule, contacting the composition with an affinity matrix comprising a photoswitchable compound in stable association with an affinity ligand and a solid support to form an affinity matrix, for a time sufficient to allow specific binding of the target molecule to the affinity matrix, washing the affinity matrix with a washing solution to remove the components of the composition not specifically bound to the affinity matrix, irradiating the affinity matrix with (a) wavelength(s) of light of at least about 400 nm in order to cause a loss of specific binding of the affinity matrix to the target molecule, and eluting the target molecule from the affinity matrix with an eluent.

According to the eighth aspect of the present invention, a method for isolating and/or purifying a target molecule is provided, wherein the method comprises the steps of providing a composition comprising a target molecule, contacting the composition with an affinity matrix comprising a photoswitchable azobiaryl compound comprising at least two substituted biphenyl moieties, preferably the photoswitchable azobiaryl compound according to the first aspect of the invention in stable association with an affinity ligand, wherein the photoswitchable azobiaryl compound and the affinity ligand are in stable association with a solid support to form an affinity matrix; or a photoswitchable affinity ligand according to the second aspect of the invention in stable association with a solid support to form an affinity matrix; or a photoswitchable affinity matrix according to the third aspect of the present invention, for a time sufficient to allow specific binding of the target molecule to the affinity matrix, washing the affinity matrix with a washing solution to remove the components of the composition not specifically bound to the affinity matrix, changing the irradiation of the affinity matrix with (a) particular wavelength(s) of light in order to cause a loss of specific binding or affinity of the affinity matrix to the target molecule, and eluting the target molecule from the affinity matrix with an eluent.

### DESCRIPTION OF FIGURES

FIG. 1 shows photoswitchable azobiaryl compounds according to the present invention. (A) Basic chemical structure of the photoswitchable azobiaryl compound, comprising an azobenzene core and peripheral groups R¹, R², R³ and R^{1′}, R^{2′}, R^{3′} defined according to the present invention; (B) Chemical structure of a preferred photoswitchable azobiaryl compound 4′-(2-(4-(2-iodoacetamido)-3′,5′-dimethoxybiphenyl-3-ylcarboxyamido)acetic acid)-4′-(2-(4-(2-iodoacetamido)-3′,5′-dimethoxybiphenyl-3-ylcarboxyamido)acetic acid)diazene referred to herein as PS1; (C) Distance of atoms in the p-position of PS1 in the stretched trans-state (24.8 A), and (D) in the more compact cis-state (12.9 A).

FIG. 2 shows the synthesis of (A) the photoswitchable azobiaryl compound PS1 and (B) the Mn^{III} SalophenCl catalysator.

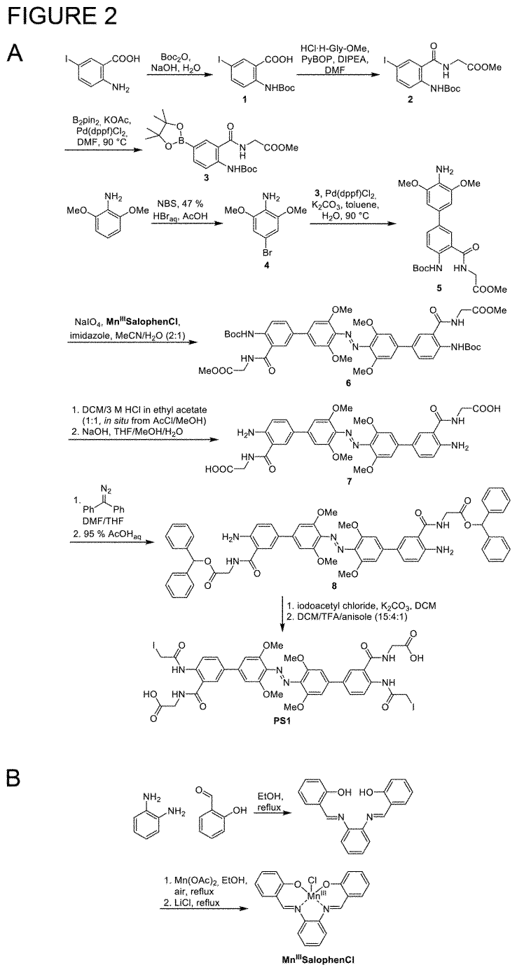

FIG. 3 shows (A) coupling of photoswitchable azobiaryl compound PS1 with N-acetyl cysteines, and photo-switching of the reaction product upon alternating irradiation cycles, (B) HPLC analysis of PS1 coupled to N-acetyl cysteine after photo-switching (isomerization) with 465 nm (blue) and 635 nm (red) irradiation, and (C) UV-VIS spectra of the photoswitchable azobiaryl compound PS1 coupled to N-acetyl cysteine in aqueous buffer (solid line: trans isomer; dotted line: cis-isomer).

FIG. 4 shows the ESI-MS spectrum of unmodified SpA #1 (upper panel) and PS1 modified SpA #1 (lower panel).

FIG. 5 shows (A) light-controlled affinity chromatography of an IgG sample using SpA #1-PS1; (B) SDS-PAGE-Analysis of the raw material and the elution fraction. Both samples showed characteristic bands for the heavy (approx. 50 kDa) and light (approx. 25 kDa) chain of IgG molecules, indicating a specific binding and subsequent, light-controlled elution of immunoglobulin G. The isolated IgG was also analyzed by an analytical SEC (C) and nano differential scanning fluorimetry (nanoDSF) (D) using a Tycho NT.6 (NanoTemper Technologies). The results showed the high quality of the protein after purification with the SpA #1-PS1 affinity matrix. (E) The proposed concept of a light-controlled affinity modulation. The effect of affinity alteration is most likely a consequence of the distortion of the protein ligand secondary structure upon the photoisomerization of the photoswitch PS1.

FIG. 6 shows chromatographic profiles illustrating the affinity purification of an immunoglobulin G (IgG) mixture (Cutaquig®) employing light-switchable protein variants: (A) The chromatogram for the purification process using the Protein G variant, SpG #1, modified with the azo-biaryl based photoswitch PS2; (B) The chromatogram for the purification process using the Protein L variant, PpL #1, also modified with PS2. Both panels demonstrate the application of samples and subsequent washing steps under red light illumination. The elution of specifically bound IgG molecules is initiated by blue light illumination, as denoted by an arrow in each chromatogram. This figure effectively illustrates the controlled manipulation of binding and release events in the purification process through light-responsive protein engineering.

FIG. 7 shows (A) HPLC analysis demonstrating the photo-switching behavior of the azobiaryl compound PS4 across successive irradiation cycles, including periods in the dark, following exposure to 593 nm (yellow light), 450 nm (blue light), and 312 nm (UV light) irradiations. This analysis illustrates the reversible transitions of PS4 under different wavelengths of light, highlighting its photo-responsive characteristics. (B) UV-VIS spectra of the photoswitchable azobiaryl compound PS4, where the solid line represents the trans-isomer and the dotted line signifies the cis-isomer. These spectra provide insight into the optical properties of PS4, detailing the absorption differences between its isomeric forms, which underpin its utility in photo-switching applications.

### DETAILED DESCRIPTION OF THE INVENTION

Before the present invention is described in more detail below, it is to be understood that this invention is not limited to the particular methodology, protocols and reagents described herein as these may vary. It is also to be understood that the terminology used herein is for the purpose of describing particular embodiments only and is not intended to limit the scope of the present invention which will be limited only by the appended claims. Unless defined otherwise, all technical and scientific terms used herein have the same meanings as commonly understood by one of ordinary skill in the art.

The present invention is based on the identification of a basic chemical structure of photoswitchable azobiaryl compounds which may be stably associated with an affinity ligand in order to allow digital switching of the affinity ligand between high and low affinity to a protein of interest by using light of different wavelengths. The specific advantages of the compounds of the present invention further include solubility in aqueous solutions in comparison to similar molecules of the prior art, which are soluble only in organic solvents and thus prevent the functionalization of affinity ligands without disturbing their function.

These advantages are brought about by the structure of the photoswitchable azobenzene compounds according to the present invention, wherein R¹ and R^{1′} facilitate the bathochromic shift of the trans to cis-isomerization-wavelength, R² and R^{2′} are solubility-enhancing moieties, and R³ and R^{3′} are ligand-reactive and/or solid support-reactive moieties.

While Köhl et al. (Synthesis 2014, 46, 2376-2382) disclose azobiaryl compounds that are structurally similar to the compounds of the present invention, the compounds disclosed therein are used in a different and unrelated technical field, and also have a different substitution pattern than the compounds of the present invention, thus leading to structures distinguished from the compounds of the present invention as described herein.

Comparable to traditional methods of affinity purification, target molecules are captured effectively from a heterogeneous protein composition by the immobilized photoswitchable affinity ligand of the present invention. By employing the compound of the present invention, however, the affinity of the affinity ligand associated with the photoswitchable azobiaryl compound towards the target can be switched digitally with light of different wavelengths.

By this means, molecules can be isolated in a buffer of choice, comforting their sensitive nature and eliminating the need for buffer exchange procedures in the following process step. Furthermore, the mode of operation allows for rapid chromatography cycling with drastically reduced buffer consumption, which implies the potential for scale-up and great possible savings. Due to the elimination of a buffer-triggered elution step and the fast regeneration of the affinity matrix after elution, the virtual binding capacity and productivity of the photoswitchable affinity chromatography system is clearly superior to processes known in the art.

Host cell protein (HCP) contaminant clearance is a significant concern during affinity chromatography (Wolter, T., & Richter, A. (2005). Assays for controlling host-cell impurities in biopharmaceuticals. BioProcess Int, 3(2), 40-46). Conventionally, an intermediate pH wash is employed between column loading and elution to minimize HCP levels during elution. HCP contaminants that co-elute at low pH comprise species that associate with the product and/or with the chromatographic resin. By eliminating the step of altering buffer conditions, only specifically bound target molecules are eluted using a light-switchable chromatography matrix.

Photoswitches are molecules that are capable of being reversibly interconverted between (at least) two states by means of light irradiation. Azobenzenes remain one of the most popular photoswitches owing to their stability, reliability and tunability: azobenzenes provide high extinction coefficients and quantum yields, allowing switching between cis- and trans-isomers with low intensity light, and are stable to repeated switching cycles.

There are several performance metrics that may be used to judge azobenzene-cores as molecular photoswitches, when it comes to the optical control of affinity matrices:
Wavelength for cis- to trans- and trans- to cis-isomerization
Thermal stability of the cis-isomer
Completeness of switching at a given wavelength
Degree of change in end-to-end distance upon photoisomerization (shape variation)

Most of the azobenzene-based photoswitches developed to date require the use of UV light for photoisomerization from the trans to the cis-state. This limits their use in applications such as affinity chromatography outside of research and on an industrial level and larger scales, mainly because UV light is strongly scattered, making penetration of materials used as solid support, e.g., an agarose bead or a polymer-based membrane, difficult. Furthermore, the more-energetic UV light can trigger degradation and chemical modification of these materials.

Also, photoswitches as known and previously explored in the art appear to only be soluble in polar to non-polar organic solvents and not in aqueous solutions. Clearly, this makes the known photoswitches unusable in aqueous environments which are commonly employed in purification of therapeutics, such as therapeutic antibodies, wherein crude extracts, affinity ligands as well as the proteins of interest are water-soluble.

A shift of the wavelength for isomerization towards the visible region (bathochromic shift) is therefore desirable. From the aspect of energy, the trans-isomer of azobenzene compounds has a lower intrinsic energy than the cis-isomer. Thus, the azo structure recovers spontaneously from the cis to the trans-state. Accordingly, the cis-state has a worse thermostability and shorter lifetime due to its higher energy state.

A short-lived cis-isomer means that an intense light source would be required in order to maintain a substantial fraction of the cis-isomer, an undesirable limitation for application in affinity chromatography. In order to achieve high cis to trans-isomerization efficiency, the two isomers of an azobenzene derivative must offer well-separated absorption bands. Most frequently, trans to cis-photoisomerization is achieved by irradiating in the region of the high-energy π-π* band for the trans-isomer, whereas cis to trans-photoisomerization occurs through irradiation in the low-energy π-π* band of the cis-isomer (Merino, E., & Ribagorda, M. (2012). Control over molecular motion using the cis-trans photoisomerization of the azo group. Beilstein journal of organic chemistry, 8(1), 1071-1090).

However, an overlap in the absorbances between cis- and trans-isomers causes incomplete photoswitching. In order to exploit and/or amplify the end-to-end distance upon photoisomerization to a greater length scale, it is vital to embed the azobenzene-core in a conformationally rigid molecular scaffold. To this end, a viable strategy consists of including them into rigid aromatic structures, e.g., a biaryl.

The wavelength for cis to trans-isomerization, the thermal stability, the photoconversion, and the shape variation strongly depend on the substitution pattern of the azobenzene-core.

Azobenzene photoswitches have previously been reported as being utilized for organic synthesis (Wolf, E., & Cammenga, H. K. (1977). Thermodynamic and kinetic investigation of the thermal isomerization of cis-azobenzene. Zeitschrift für Physikalische Chemie, 107 (1), 21-38), functional materials including self-healing materials (Suginome, H. (2004). CRC Handbook of Organic Photochemistry and Photobiology), adhesives (Morgenstern, K. (2009). Isomerization reactions on single adsorbed molecules. Accounts of chemical research, 42 (2), 213-223), photoresists (Pace, G., Ferri, V., Grave, C., Elbing, M., von Hanisch, C., Zharnikov, M., . . . & Samorì, P. (2007). Cooperative light-induced molecular movements of highly ordered azobenzene self-assembled monolayers. Proceedings of the National Academy of Sciences, 104 (24), 9937-9942), and optical materials (Choi, B. Y., Kahng, S. J., Kim, S., Kim, H., Kim, H. W., Song, Y. J., . . . & Kuk, Y. (2006). Conformational molecular switch of the azobenzene molecule: a scanning tunneling microscopy study. Physical review letters, 96 (15), 156106). In these applications, they have been employed in solid/liquid states or as solutions in organic solvents.

However, there is a particular need for, and interest in, photoswitches that are functional in aqueous solution, for example, for modulation of biological activities. Because of the essentially hydrophobic character of azobenzene derivates, modification of molecules with such photoswitches was limited to reactions that may be carried out in organic solvents or organic solvent/water mixtures.

Sensitive biomolecules such as polypeptides or proteins may however irreversibly unfold under these conditions. As the intact 3D-structure of a polypeptide or protein is a requirement to address the correct attachment positions, aqueous buffer systems are needed to enable stable association of a photoswitch. This technical problem is solved by the compound of the present invention introducing a solubility-enhancing moiety as described herein. The solubility-enhanced photoswitchable azobiaryl compounds have good solubility in aqueous solutions at physiological pH, which represents one of the technical advantages of the present invention.

Accordingly, the photoswitchable azobiaryl compound which is defined by formula (I) as provided herein has the advantages that the critical aspects discussed above can be addressed to match the challenging demand for affinity chromatography by the selection of appropriate substituents.

Thus, in the first aspect of the invention, a photoswitchable azobiaryl compound is provided according to formula (I)

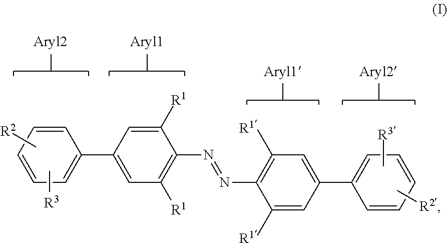

wherein R¹ and R^{1′} are independently selected from the group comprising H, F, Cl, Br, (CH₂)ₙCH₃, CH((CH₂)ₙCH₃)((CH₂)ₓCH₃), C((CH₂)ₙCH₃)((CH₂)ₓCH₃)((CH₂)_{z}CH₃), (CH₂)ₙCH((CH₂)ₓCH₃)((CH₂)_{z}CH₃), (CH₂)ₙC((CH₂)ₓCH₃)((CH₂)_{y}CH₃)((CH₂)_{z}CH₃), O(CH₂)ₙCH₃, OCH((CH₂)ₙCH₃)((CH₂)ₓCH₃), OC((CH₂)ₙCH₃)—((CH₂)ₓCH₃)((CH₂)_{z}CH₃), O(CH₂)ₙCH((CH₂)ₓCH₃)((CH₂)_{z}CH₃), O(CH₂)ₙC((CH₂)ₓCH₃)—((CH₂)_{y}CH₃)((CH₂)_{z}CH₃), S(CH₂)ₙCH₃, SCH((CH₂)ₙCH₃)((CH₂)ₓCH₃), SC((CH₂)ₙCH₃)((CH₂)ₓCH₃)—((CH₂)_{z}CH₃), S(CH₂)ₙCH((CH₂)ₓCH₃)((CH₂)_{z}CH₃), S(CH₂)ₙC((CH₂)ₓCH₃)((CH₂)_{y}CH₃)((CH₂)_{z}CH₃), NH₂, NH(CH₂)ₙCH₃, NHCH((CH₂)ₙCH₃)—((CH₂)ₓCH₃), NHC((CH₂)ₙCH₃)((CH₂)ₓCH₃)((CH₂)_{z}CH₃), NH(CH₂)ₙCH((CH₂)ₓCH₃)((CH₂)_{z}CH₃), NH(CH₂)ₙC—((CH₂)ₓCH₃)—((CH₂)_{y}CH₃)((CH₂)_{z}CH₃), N((CH₂)ₙCH₃)₂, N(CH((CH₂)ₙCH₃)((CH₂)ₓCH₃))₂, N(C((CH₂)ₙCH₃)—((CH₂)ₓCH₃)((CH₂)_{z}CH₃))₂, N((CH₂)ₙCH((CH₂)ₓCH₃)((CH₂)_{z}CH₃))₂, N((CH₂)ₙC((CH₂)ₓCH₃)((CH₂)_{y}CH₃)—((CH₂)_{z}CH₃))₂, aziridin-1-yl, azetidin-1-yl, pyrrolidin-1-yl, piperidin-1-yl, azepan-1-yl, azocan-1yl, morpholin-1-yl, 4-methyl-piperazin-1-yl, wherein n may be any integer from 0 to 10, wherein x may be any integer from 0 to 10, wherein y may be any integer from 0 to 10 and wherein z may be any integer from 0 to 10;
R² and R^{2′} are independently selected from the group comprising SO₃H, SO₃Li, SO₃Na, SO₃K, COOH, COONHS, CONH₂, CONH—(CH₂)ₙCH₃, CONH—((CH₂)ₙCH₃)((CH₂)_{z}CH₃), CONH—(CH₂)ₙSO₃H, CONH—(CH₂)ₙSO₃Li, CONH—(CH₂)ₙSO₃Na, CONH—(CH₂)ₙSO₃K, CONH—(CH₂)ₙCCH, CONH—(CH₂)ₙN₃, CONH—(CH₂)ₙNH₂, CONH—(CH₂)ₙCOOH, CONH—(CH₂)ₙNHAcl, CONH—(CH₂)ₙNHAcBr, CONH—(CH₂)ₙNHAcCl, CONH—(CH₂)ₙN(maleimide), CONH—(CH₂)ₙ—NH(2-chloromethyl acrylate), CONH—(CH₂)ₙ—NH(vinylsulfonate), CONH—(CH₂)ₙ—NHCOPhF₅, CONH—(CH₂)ₙ—NHSO₂PhF₅, CONH—(CH₂)ₙCOONHS, CONH-PEG-OH, CONH-PEG-NH₂, CONH-PEG-COOH, CONH-PEG-COONHS, CONH-PEG-O(CH₂)ₙSO₃H, CONH-PEG-O(CH₂)ₙSO₃Li, CONH-PEG-O(CH₂)ₙSO₃Na, CONH-PEG-O(CH₂)ₙSO₃K, CONH-PEG-NHAcl, CONH-PEG-NHAcBr, CONH-PEG-NHACCI, CONH-PEG-N(maleimide), CONH—PEG-NH(2-chloromethyl acrylate), CONH-PEG-NH(vinylsulfonate), CONH-PEG-NHCOPhF₅, CONH—PEG-NHSO₂PhF₅, CONH-PEG-N₃, CONH-PEG-OCH₂CCH, CONH-PEG-NHCH₂CCH, CONH-PEG-N(CH₂CCH)₂, CONH-PEG-NHCO(CH₂)ₙCOOH, CONH-PEG-NHCO(CH₂)ₙCOONHS, CONH—(Xaa) n OH, CONH—(Xaa)ₙ-NH(CH₂)ₙSO₃H, CONH—(Xaa)ₙ-NH(CH₂)ₙSO₃Li, CONH—(Xaa)ₙ-NH(CH₂)ₙSO₃Na, CONH—(Xaa)ₙ-NH(CH₂)ₙSO₃K, CONH—(Xaa)ₙ-OMe, CONH—(Xaa)ₙ-ONHS, CONH—(Xaa)ₙ-ONHS, CONH—(Xaa)ₙ-NH(CH₂)ₙCCH, CONH—(Xaa)ₙ-NH(CH₂)ₙN₃, CONH—(Xaa)ₙ-NH(CH₂)_{z}NHAcl, CONH—(Xaa)ₙ-NH(CH₂)_{z}NHAcBr, CONH—(Xaa)ₙ-NH(CH₂)_{z}NHAcCI, CONH—(Xaa)ₙ-NH(CH₂)_{z}—N(maleimide), CONH—(Xaa)ₙ-NH(CH₂)_{z}NH(2-chloromethyl acrylate), CONH—(Xaa)ₙ-NH(CH₂)_{z}NH(vinylsulfonate), CONH—(Xaa)ₙ-NH(CH₂)_{z}NHCOPhF₅, CONH—(Xaa)ₙ-NH(CH₂)_{z}NHSO₂PhF₅, CONH—(Xaa)ₙ-N₃, CONH—(Xaa)ₙ-OCH₂CCH, CONH—(Xaa)ₙ-NHCH₂CCH, CONH—(Xaa)ₙ-N(OCH₂CCH)₂, CONH—(Xaa)ₙ-NH—(CH₂)ₙCOOH, CONH—(Xaa)ₙ-NH—(CH₂)ₙCOONHS, CONH—(Xaa)ₙ-NH-PEG-OH, CONH—(Xaa)ₙ-NH-PEG-NH₂, CONH—(Xaa)ₙ-NH-PEG-COOH, CONH—(Xaa)ₙ-NH-PEG-COONHS, CONH—(Xaa)ₙ-NH-PEG-NHAcl, CONH—(Xaa)ₙ-NH-PEG-NHAcBr, CONH—(Xaa)ₙ-NH-PEG-NHACCI, CONH—(Xaa)ₙ-NH-PEG-N(maleimide), CONH—(Xaa)ₙ-NH-PEG-NH(2-chloromethyl acrylate), CONH—(Xaa)ₙ-NH-PEG-NH(vinylsulfonate), CONH—(Xaa)ₙ-NH-PEG-NHCOPhF₅, CONH—(Xaa)ₙ-NH-PEG-NHSO₂PhF₅, CONH—(Xaa)ₙ-NH-PEG-N₃, CONH—(Xaa)ₙ-NH-PEG-OCH₂CCH, CONH—(Xaa)ₙ-NH-PEG-NHCH₂CCH, CONH—(Xaa)ₙ-NH-PEG-N(CH₂CCH)₂, CONH—(Xaa)ₙ-NH-PEG-NHCO(CH₂)ₙCOOH, CONH—(Xaa)ₙ-NH-PEG-NHCO(CH₂)ₙCOONHS, wherein Xaa can be any canonical or non-canonical amino acid, wherein n may be any integer from 0 to 10, and wherein z may be any integer from 0 to 10;
R³ and R^{3′} are independently selected from the group comprising H, NH₂, NHCO (C₁-C₆-alkyl), NHCO(C₁-C₆-haloalkyl), NHBoc, NHCbz, NHalloc, NH(CH₂)ₙCH₃, NHCH((CH₂)ₙCH₃)((CH₂)ₓCH₃), NHC((CH₂)ₙCH₃)((CH₂)ₓCH₃)((CH₂)_{z}CH₃), NH(CH₂)ₙCH((CH₂)ₓCH₃)((CH₂)_{z}CH₃), NH(CH₂)ₙC((CH₂)ₓCH₃)((CH₂)_{y}CH₃)((CH₂)_{z}CH₃), N((CH₂)ₙCH₃)₂, N(CH((CH₂)ₙCH₃)—((CH₂)ₓCH₃))₂, N(C((CH₂)ₙCH₃)((CH₂)ₓCH₃)((CH₂)_{z}CH₃))₂, N((CH₂)ₙCH((CH₂)ₓCH₃)—((CH₂)_{z}CH₃))₂, N((CH₂)ₙC((CH₂)ₓCH₃)((CH₂)_{y}CH₃)((CH₂)_{z}CH₃))₂, aziridin-1-yl, azetidin-1-yl, pyrrolidin-1-yl, piperidin-1-yl, azepan-1-yl, azocan-1yl, morpholin-1-yl, 4-methyl-piperazin-1-yl, NHCH₂CHCH₂, N(CH₂CHCH₂)₂, NHCH₂CCH, N(CH₂CCH)₂, NHCOCCH, NHCO(CH₂)ₙN₃, NHacrylate, NH(2-chloromethyl acrylate), NH(vinyl sulfonate), N(maleimide), N(2-bromomaleimide), N(2,3-dibromomaleimide), NHCOaryl, NHCOhaloaryl, NHSO₂aryl, NHSO₂haloaryl, (CH₂)ₙN(maleimide), (CH₂)ₙN(2-bromomaleimide), (CH₂)ₙN(2,3-dibromomaleimide), NHCO(CH₂)ₙN(maleimide), NHCO(CH₂)ₙN(2-bromomaleimide), NHCO(CH₂)ₙN(2,3-dibromomaleimide), NCS, NCO, NH(CH₂)ₙCH(O)CH₂, N((CH₂)ₙCH(O)CH₂)₂, NACCH₂CH(O)CH₂ wherein n may be any integer from 0 to 10, and wherein z may be any integer from 0 to 10.

In a preferred embodiment, at least one of R¹ or R^{1′} is not H.

In one embodiment of the first aspect of the invention,
R¹ and R^{1′} are independently selected from the group comprising H, F, Cl, Br, (CH₂)ₘCH₃, CH((CH₂)ₙCH₃)((CH₂)ₓCH₃), C((CH₂)ₙCH₃)((CH₂)ₓCH₃)((CH₂)_{z}CH₃), (CH₂)ₙCH((CH₂)ₓCH₃)((CH₂)_{z}CH₃), (CH₂)ₙC((CH₂)ₓCH₃)((CH₂)_{y}CH₃)((CH₂)_{z}CH₃), O(CH₂)ₙCH₃, OCH((CH₂)ₙCH₃)((CH₂)ₓCH₃), OC((CH₂)ₙCH₃)—((CH₂)ₓCH₃)((CH₂)_{z}CH₃), O(CH₂)ₙCH((CH₂)ₓCH₃)((CH₂)_{z}CH₃), O(CH₂)ₙC((CH₂)ₓCH₃)—((CH₂)_{y}CH₃)((CH₂)_{z}CH₃), S(CH₂)ₙCH₃, SCH((CH₂)ₙCH₃)((CH₂)ₓCH₃), SC((CH₂)ₙCH₃)((CH₂)ₓCH₃)—((CH₂)_{z}CH₃), S(CH₂)ₙCH((CH₂)ₓCH₃)((CH₂)_{z}CH₃), S(CH₂)ₙC((CH₂)ₓCH₃)((CH₂)_{y}CH₃)((CH₂)_{z}CH₃), NH₂, NH(CH₂)ₙCH₃, NHCH((CH₂)ₙCH₃)—((CH₂)ₓCH₃), NHC((CH₂)ₙCH₃)((CH₂)ₓCH₃)((CH₂)_{z}CH₃), NH(CH₂)ₙCH((CH₂)ₓCH₃)((CH₂)_{z}CH₃), NH(CH₂)ₙC—((CH₂)ₓCH₃)—((CH₂)_{y}CH₃)((CH₂)_{z}CH₃), N((CH₂)ₙCH₃)₂, N(CH((CH₂)ₙCH₃)((CH₂)ₓCH₃))₂, N(C((CH₂)ₙCH₃)—((CH₂)ₓCH₃)((CH₂)_{z}CH₃))₂, N((CH₂)ₙCH((CH₂)ₓCH₃)((CH₂)_{z}CH₃))₂, N((CH₂)ₙC((CH₂)ₓCH₃)((CH₂)_{y}CH₃)—((CH₂)_{z}CH₃))₂, aziridin-1-yl, azetidin-1-yl, pyrrolidin-1-yl, piperidin-1-yl, azepan-1-yl, azocan-1yl, morpholin-1-yl, 4-methyl-piperazin-1-yl, wherein m may be any integer from 1 to 10, wherein n may be any integer from 0 to 10, wherein x may be any integer from 0 to 10, wherein y may be any integer from 0 to 10, and wherein z may be any integer from 0 to 10.

In one embodiment of the first aspect of the invention,
R³ and R^{3′} are independently selected from the group comprising H, NH₂, NHAc, NHBoc, NHCbz, NHalloc, NHTfAc, NH(CH₂)ₙCH₃, NHCH((CH₂)ₙCH₃)((CH₂)ₓCH₃), NHC((CH₂)ₙCH₃)((CH₂)ₓCH₃)—((CH₂)_{z}CH₃), NH(CH₂)ₙCH((CH₂)ₓCH₃)((CH₂)_{z}CH₃), NH(CH₂)ₙC((CH₂)ₓCH₃)((CH₂)_{y}CH₃)((CH₂)_{z}CH₃), N((CH₂)ₙCH₃)₂, N(CH((CH₂)ₙCH₃)—((CH₂)ₓCH₃))₂, N(C((CH₂)ₙCH₃)((CH₂)ₓCH₃)((CH₂)_{z}CH₃))₂, N((CH₂)ₙCH((CH₂)ₓCH₃)—((CH₂)_{z}CH₃))₂, N((CH₂)ₙC((CH₂)ₓCH₃)((CH₂)_{y}CH₃)((CH₂)_{z}CH₃))₂, aziridin-1-yl, azetidin-1-yl, pyrrolidin-1-yl, piperidin-1-yl, azepan-1-yl, azocan-1yl, morpholin-1-yl, 4-methyl-piperazin-1-yl, NHCH₂CHCH₂, N(CH₂CHCH₂)₂, NHCH₂CCH, N(CH₂CCH)₂, NHCOCCH, NHCO(CH₂)ₙN₃, NHACCI, NHAcBr, NHAcl, NHacrylate, NH(2-chloromethyl acrylate), NH(vinyl sulfonate), N(maleimide), N(2-bromomaleimide), N(2,3-dibromomaleimide), NHCOPhF₅, NHSO₂PhF₅, (CH₂)ₙN(maleimide), (CH₂)ₙN(2-bromomaleimide), (CH₂)ₙN(2,3-dibromomaleimide), NHCO(CH₂)ₙN(maleimide), NHCO(CH₂)ₙN(2-bromomaleimide), NHCO(CH₂)ₙN(2,3-dibromomaleimide), NCS, NCO, NH(CH₂)ₙCH(O)CH₂, N((CH₂)ₙCH(O)CH₂)₂, NACCH₂CH(O)CH₂ wherein n may be any integer from 0 to 10, and wherein z may be any integer from 0 to 10.

The term “alkyl” as used herein denotes in each case a straight-chain or branched alkyl group having usually from 1 to 6 carbon atoms, preferably 1 to 4 carbon atoms, more preferably 1 to 3 or 1 or 2 carbon atoms. Examples of an alkyl group are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-di-methylpropyl, 1-ethyl propyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethyl-butyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl.

The term “haloalkyl” as used herein denotes in each case a straight-chain or branched alkyl group having usually from 1 to 6 carbon atoms, frequently 1 to 4 carbon atoms, preferably 1 to 3 or 1 or 2 carbon atoms, wherein the hydrogen atoms of this group are partially or totally replaced with halogen atoms such as fluorine, bromine, chlorine or iodine. Preferred haloalkyl moieties are selected from C₁-C₄-haloalkyl, more preferably from C₁-C₃-haloalkyl or C₁-C₂-haloalkyl, in particular from C₁-C₂-fluoroalkyl such as fluoromethyl, bromomethyl, chloromethyl, iodomethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “aryl” as used herein refers to an “aromatic ring system” (i.e. fulfilling the Hückel rule-having (4n+TT2) electrons, with n being 0 or an integer of preferably 1 to 3). More specifically, those aromatic ring systems may be mono-, bi- or tricyclic with 5, 6, 7, 8, 9, 10, 11, 12, 13, 14 ring carbon atoms. Even more specifically, those aromatic ring systems may be monocyclic with 6 ring carbon atoms. Exemplary aryl groups are phenyl, biphenyl, naphthyl, anthracyl and the like.

The term “haloaryl” as used herein refers to an aryl, wherein the hydrogen atoms of this group are partially or totally replaced with halogen atoms such as fluorine, bromine, chlorine or iodine. Exemplary haloaryl are pentafluorophenyl, monofluorophenyl(ortho, meta, para), and the like.

According to the present invention, independent of the presentation of a compound in its cis or trans state, the other isomeric state is also considered to be envisaged unless expressly indicated to the contrary.

In a preferred embodiment of the first aspect of the invention, Aryl¹-Aryl² and Aryl¹′-Aryl^{2′} are multifunctionalized biaryl compounds, more preferably wherein at least one R¹ and at least one R^{1′} is not hydrogen, even more preferably wherein both R¹ and both R^{1′} are not hydrogen. In a preferred embodiment of the first aspect of the invention, at least one of R¹ or R^{1′} is not methyl such as at least one R¹ and at least one R^{1′} is not methyl or both R¹ and both R^{1′} are not methyl. Preferably, Aryl¹-Aryl² may carry at most four substituents, more preferably exactly four substituents. Also preferably, Aryl^{1′}-Aryl^{2′} may carry at most four substituents, more preferably exactly four substituents. Particularly preferably, each biaryl may carry at most four substituents, more preferably exactly four substituents.

In one preferred embodiment, R¹ and R^{1′} are independently selected from the group comprising alkoxy or halogen, more preferably from the group comprising methoxy, ethoxy, chlor or fluor.

In another preferred embodiment of the first aspect of the invention, at least one, preferably both, of R² and R^{2′} is CONH-Xaa-OH (Xaa=any canonical or non-canonical amino acid), or at least one, preferably both, of R² and R^{2′} comprise a linker selected from the group consisting of peptides, bifunctional alkanes, poly(alkylene oxides), more preferably wherein said poly(alkylene oxides) has a molecular weight selected from the group consisting of between about 100 g/mol and about 80,000 g/mol, even more preferably between about 100 g/mol and 6,000 g/mol. In another preferred embodiment of the first aspect of the invention, at least one of R² or R^{2′} is not COOH, preferably wherein both R² and R^{2′} are not COOH.

In one preferred embodiment, R² and/or R^{2′} may be an amide or peptide, more preferably-CONH—(Xaa)ₙ-OH, even more preferably —COHNH—CH₂—COOH).

In one preferred embodiment of the first aspect of the invention, at least one, preferably both, of R³ and R^{3′} is a substituent selected from the group comprising amines, acrylamides, NHCO(C₁-C₆-haloalkyl) such as α-haloacetamides, vinyl sulfonates, isothiocyanates, isocyanates, epoxides, maleimides, haloaryl carboxy- and haloaryl sulfonamides such as fluorophenyl carboxy- and fluorophenyl sulfonamides.

Preferably, R³ and/or R^{3′} is generally a substituent with reactivity to one or more of an affinity ligand, a solid support, a polymer, a polypeptide, an oligonucleotide, a polynucleotide, a nucleic acid, a carbohydrate, a liposome, a nanoparticle, a cell, a biomacromolecule, a biomolecule or a small molecule.

The ligand-reactive moiety of R³ and/or R^{3′} is preferably any of a variety of reactive groups that provides for stable association of the photoswitchable azobiaryl compound to an affinity ligand. Stable association of the photoswitchable azobiaryl compound to an affinity ligand includes covalent linkage; as well as non-covalent associations such as ionic interactions, and the like. Preferably, stable association may be caused by a covalent bond.

In general, where the stable association is a non-covalent association, the stable association is a high-affinity association. Suitable ligand-reactive moieties may preferably be a maleimide, an acrylic amide (an acrylamide), an α-haloacetamide, an epoxide, an O-succinimidyl ester, a fluorophenyl sulfonamide and a fluorophenyl carboxyamide.

In a further embodiment, the photoswitchable azobiaryl compound provides for intermolecular cross-linking of the affinity ligands to from a homodimer or multimer by two ligand-reactive moieties linked via an azobenzene-core in a homofunctional (R³═R^{3′}) or heterofunctional (R³≠R^{3′}) manner.

In another embodiment, the photoswitchable azobiaryl compound provides for stable association of the affinity ligand to a solid support, a polymer, a polypeptide, an oligonucleotide, a polynucleotide, a nucleic acid, a carbohydrate, a liposome, a nanoparticle, a cell, a biomacromolecule, a biomolecule or a small molecule by ligand-reactive moieties linked via an azobenzene-core in a homofunctional (R³═R^{3′}) or heterofunctional (R³≠R^{3′}) manner.

In some embodiments, the ligand-reactive moieties provide for covalent linkage with at least one amino acid side chain in a polypeptide. Linkage of the photoswitchable azobiaryl compound to an affinity ligand can be via a tyrosine residue, a tryptophan residue, a serine residue, a threonine residue, cysteine residue, a histidine residue, an arginine residue, a lysine residue, an aspartic acid residue, a glutamic acid residue, or any canonical or non-canonical amino acid in the polypeptide that is accessible for reacting with the ligand-reactive moiety of the photoswitchable azobiaryl compound, preferably via a cysteine, histidine, lysine or methionine residue.

In one embodiment, for example, where the amino acid to which the photoswitchable azobiaryl compound is to be linked is a cysteine residue, the ligand-reactive moiety comprises a group such as, e.g., an α-haloacetamide, a vinylsulfone group, fluorophenyl carboxyamide, fluorophenyl sulfonamide, maleimide, epoxide or a substituted maleimide (e.g., NHCOCH₂Cl, NHCOCH₂Br, NHCOCH₂I, NH(vinyl sulfonate), N(maleimide), N(2-bromomaleimide), N(2,3-dibromomaleimide), NHCOPhF₅, NHSO₂PhF₅, (CH₂)ₙN(maleimide), (CH₂)ₙN(2-bromomaleimide), (CH₂)ₙN(2,3-dibromomaleimide), NHCO(CH₂)ₙN(maleimide), NHCO(CH₂)ₙN(2-bromomaleimide), NHCO(CH₂)ₙN(2,3-dibromomaleimide, NHCH₂CH(O)CH₂, N(CH₂CH(O)CH₂)₂)) wherein n may be any integer from 0 to 10.

Where the amino acid to which the photoswitchable azobiaryl compound is to be linked is a lysine residue, the ligand-reactive moiety comprises in some embodiments a group such as, e.g., an active ester like N-hydroxysuccinimidyl ester (generated by N-hydroxysuccinimide and EDC), epoxide, isothiocyanate or isocyanate.

Preferably, R³ and R^{3′} are able to form an intramolecular linkage within an affinity ligand via amino acid side chains, more preferably comprising a cysteine residue, a histidine residue, a lysine residue, a methionine residue or any canonical or non-canonical amino acid in the polypeptide of the affinity ligand that is accessible for reacting with the ligand-reactive moiety of the photoswitchable azobiaryl compound.

For example, in some embodiments, R³ and R^{3′} are thiol-reactive moieties covalently linked via the azobenzene core, characterized in that the thiol-reactive moieties comprise a reactive electrophile for reaction with a nucleophile of the affinity ligand. In one preferred embodiment, intramolecular linkage within the affinity ligand is caused in a heterofunctional manner (R³≠R^{3′}). In another preferred embodiment, intramolecular linkage within the affinity ligand is caused in a homofunctional manner (R³ ═R³).

Also preferably, R³ and R^{3′} are able to form an intermolecular linkage between an affinity ligand and a second affinity ligand and/or a solid support via amino acid side chains, more preferably involving a cysteine residue, a histidine residue, a lysine residue, a methionine residue or any canonical or non-canonical amino acid in the polypeptide or functionality of the solid support that is accessible for reacting with the ligand-reactive moiety of the photoswitchable azobiaryl compound.

In one preferred embodiment, R³ and/or R^{3′} may be a haloacetamide, more preferably iodoacetamide or chloroacetamide.

In one preferred embodiment, if R³ and R^{3′} are both hydrogen, at least one R² or R^{2′} is not COOH.

In one preferred embodiment of the first aspect of the invention, the compound is 4′-(2-(4-(2-iodoacetamido)-3′,5′-dimethoxybiphenyl-3-ylcarboxyamido)acetic acid)-4′-(2-(4-(2-iodoacetamido)-3′, 5′-dimethoxybiphenyl-3-ylcarboxyamido)acetic acid)diazene (alternatively referred to herein as PS1).

The aforementioned structure PS1 may be depicted as follows:

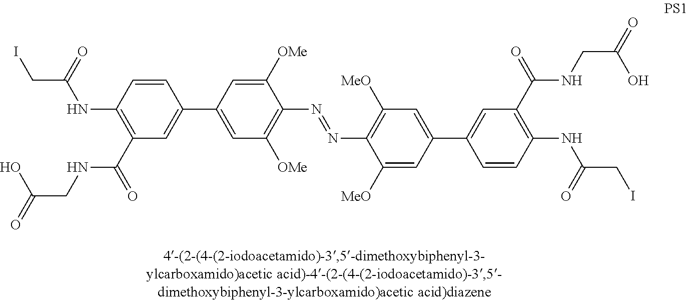 [SMILES: COc1cc(-c2ccc(NC(=O)CI)c(C(=O)NCC(=O)O)c2)cc(OC)c1/N=N/c1c(OC)cc(-c2ccc(NC(=O)CI)c(C(=O)NCC(=O)O)c2)cc1OC]

In another preferred embodiment of the first aspect of the invention, the compound is 4′-(2-(4-(2-chloroacetamido)-3′,5′-dimethoxybiphenyl-3-ylcarboxyamido)acetic acid)-4′-(2-(4-(2-chloroacetamido)-3′,5′-dimethoxybiphenyl-3-ylcarboxyamido)acetic acid)diazene (alternatively referred to herein as PS2).

The aforementioned structure PS2 may be depicted as follows:

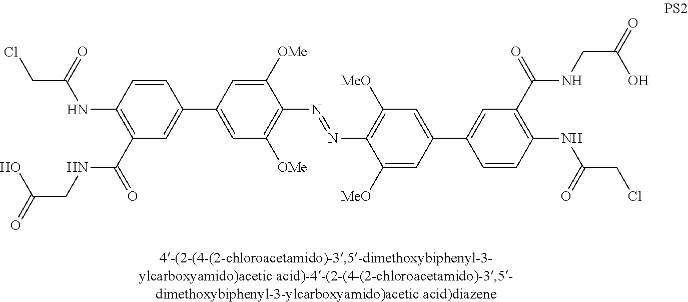 [SMILES: COc1cc(-c2ccc(NC(=O)CCl)c(C(=O)NCC(=O)O)c2)cc(OC)c1/N=N/c1c(OC)cc(-c2ccc(NC(=O)CCl)c(C(=O)NCC(=O)O)c2)cc1OC]

In another preferred embodiment of the first aspect of the invention, the compound is 4′-(2-(4-(2,5-dioxo-2,5-dihydro-1H-pyrrol-1-yl)acetamido)-3′,5′-dimethoxybiphenyl-3-ylcarboxamido)acetic acid)-4′-(2-(4-(2,5-dioxo-2,5-dihydro-1H-pyrrol-1-yl)acetamido)-3′,5′-dimethoxybiphenyl-3-ylcarboxamido)acetic acid)diazene (alternatively referred to herein as PS3).

The aforementioned structure PS3 may be depicted as follows:

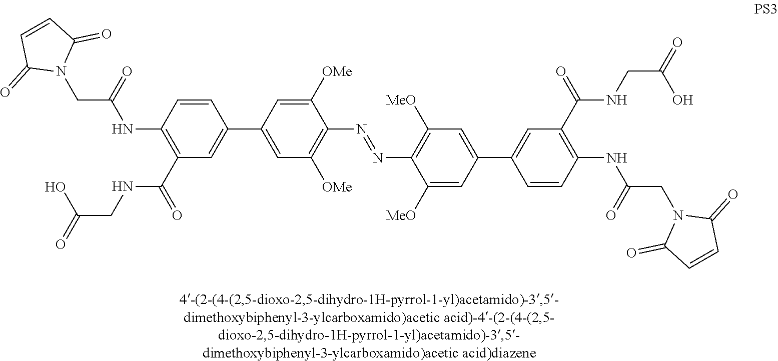 [SMILES: COc1cc(-c2ccc(NC(=O)CN3C(=O)C=CC3=O)c(C(=O)NCC(=O)O)c2)cc(OC)c1/N=N/c1c(OC)cc(-c2ccc(NC(=O)CN3C(=O)C=CC3=O)c(C(=O)NCC(=O)O)c2)cc1OC]

In another preferred embodiment of the first aspect of the invention, the compound is 4′-(2-(4-(2-bromoacetamido)-3′,5′-dimethoxybiphenyl-3-ylcarboxyamido)acetic acid)-4′-(2-(4-(2-bromoacetamido)-3′,5′-dimethoxybiphenyl-3-ylcarboxyamido)acetic acid)diazene.

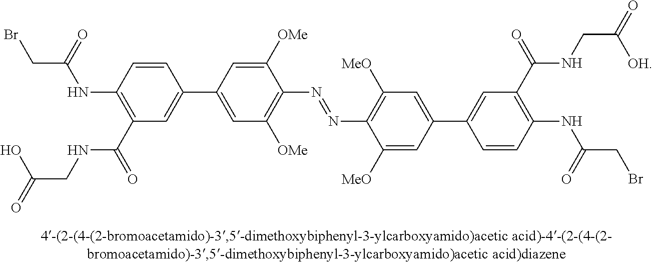 [SMILES: COc1cc(-c2ccc(NC(=O)CBr)c(C(=O)NCC(=O)O)c2)cc(OC)c1/N=N/c1c(OC)cc(-c2ccc(NC(=O)CBr)c(C(=O)NCC(=O)O)c2)cc1OC]

In another preferred embodiment of the first aspect of the invention, the compound is 4′-(2-(4-(perfluorophenyl)sulfonamido)-3′, 5′-dimethoxybiphenyl-3-ylcarboxamido)acetic acid)-4′-(2-(4-(perfluorophenyl) sulfonamido)-3′,5′-dimethoxybiphenyl-3-ylcarboxamido)acetic acid)diazene.

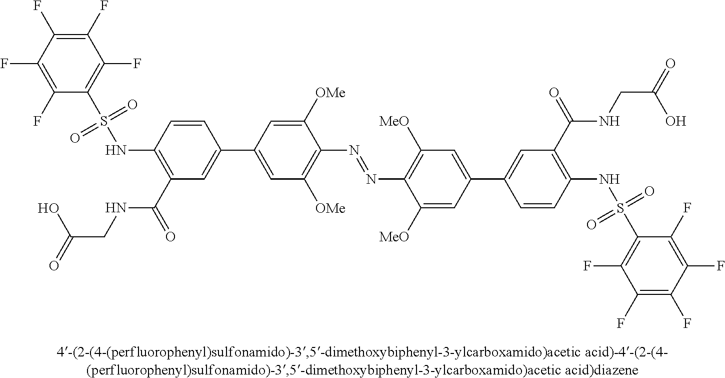 [SMILES: COc1cc(-c2ccc(NS(=O)(=O)c3c(F)c(F)c(F)c(F)c3F)c(C(=O)NCC(=O)O)c2)cc(OC)c1/N=N/c1c(OC)cc(-c2ccc(NS(=O)(=O)c3c(F)c(F)c(F)c(F)c3F)c(C(=O)NCC(=O)O)c2)cc1OC]

In another preferred embodiment of the first aspect of the invention, the compound is 4′-(2-(4-(2-chloroacetamido)-3′,5′-diethoxybiphenyl-3-ylcarboxyamido)acetic acid)-4′-(2-(4-(2-chloroacetamido)-3′,5′-diethoxybiphenyl-3-ylcarboxyamido)acetic acid)diazene.

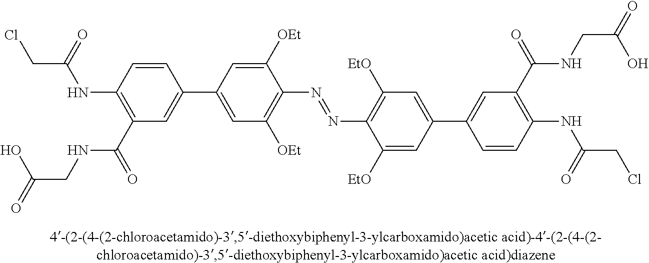 [SMILES: CCOc1cc(-c2ccc(NC(=O)CCl)c(C(=O)NCC(=O)O)c2)cc(OCC)c1/N=N/c1c(OCC)cc(-c2ccc(NC(=O)CCl)c(C(=O)NCC(=O)O)c2)cc1OCC]

In another preferred embodiment of the first aspect of the invention, the compound is 4′-(2-(4-(2-iodoacetamido)-3′,5′-difluorobiphenyl-3-ylcarboxyamido)acetic acid)-4′-(2-(4-(2-iodoacetamido)-3′,5′-difluorobiphenyl-3-ylcarboxyamido)acetic acid)diazene.

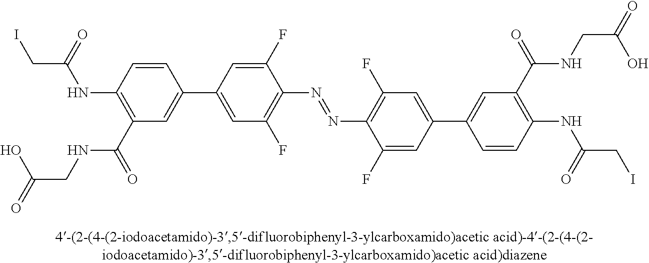 [SMILES: O=C(O)CNC(=O)c1cc(-c2cc(F)c(/N=N/c3c(F)cc(-c4ccc(NC(=O)CI)c(C(=O)NCC(=O)O)c4)cc3F)c(F)c2)ccc1NC(=O)CI]

In another preferred embodiment of the first aspect of the invention, the compound is 4′-(2-(4-(2-bromoacetamido)-3′, 5′-difluorobiphenyl-3-ylcarboxyamido)acetic acid)-4′-(2-(4-(2-bromoacetamido)-3′,5′-difluorobiphenyl-3-ylcarboxyamido)acetic acid)diazene.

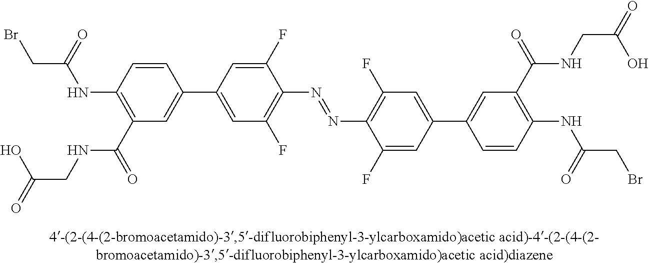 [SMILES: O=C(O)CNC(=O)c1cc(-c2cc(F)c(/N=N/c3c(F)cc(-c4ccc(NC(=O)CBr)c(C(=O)NCC(=O)O)c4)cc3F)c(F)c2)ccc1NC(=O)CBr]

In another preferred embodiment of the first aspect of the invention, the compound is 4′-(2-(4-(2-chloroacetamido)-3′,5′-difluorobiphenyl-3-ylcarboxyamido)acetic acid)-4′-(2-(4-(2-chloroacetamido)-3′,5′-difluorobiphenyl-3-ylcarboxyamido)acetic acid)diazene (alternatively referred to herein as PS4).

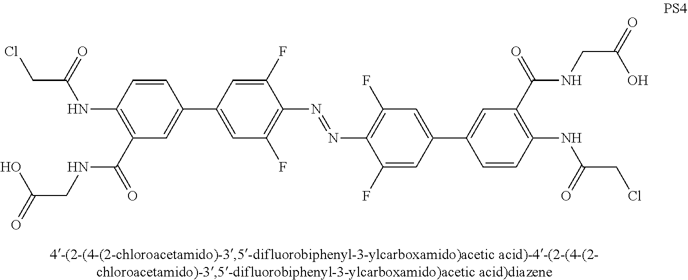 [SMILES: O=C(O)CNC(=O)c1cc(-c2cc(F)c(/N=N/c3c(F)cc(-c4ccc(NC(=O)CCl)c(C(=O)NCC(=O)O)c4)cc3F)c(F)c2)ccc1NC(=O)CCl]

In another preferred embodiment of the first aspect of the invention, the compound is 4′-(2-(4-(2,5-dioxo-2,5-dihydro-1H-pyrrol-1-yl)acetamido)-3′, 5′-difluorobiphenyl-3-ylcarboxamido)acetic acid)-4′-(2-(4-(2,5-dioxo-2,5-dihydro-1H-pyrrol-1-yl)acetamido)-3′, 5′-difluorobiphenyl-3-ylcarboxamido)acetic acid)diazene.

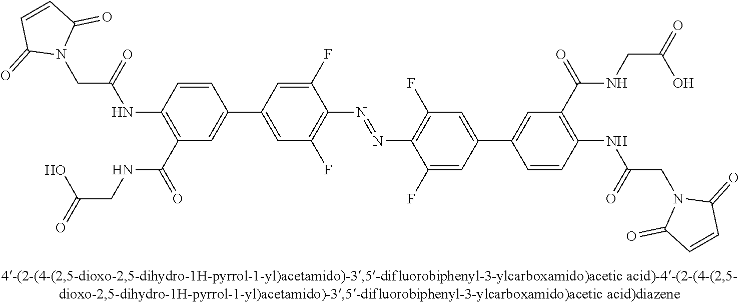 [SMILES: O=C(O)CNC(=O)c1cc(-c2cc(F)c(/N=N/c3c(F)cc(-c4ccc(NC(=O)CN5C(=O)C=CC5=O)c(C(=O)NCC(=O)O)c4)cc3F)c(F)c2)ccc1NC(=O)CN1C(=O)C=CC1=O]

In another preferred embodiment of the first aspect of the invention, the compound is 4′-(2-(4-(perfluorophenyl)sulfonamido)-3′,5′-difluorobiphenyl-3-ylcarboxamido)acetic acid)-4′-(2-(4-(perfluorophenyl) sulfonamido)-3′,5′-difluorobiphenyl-3-ylcarboxamido)acetic acid)diazene.

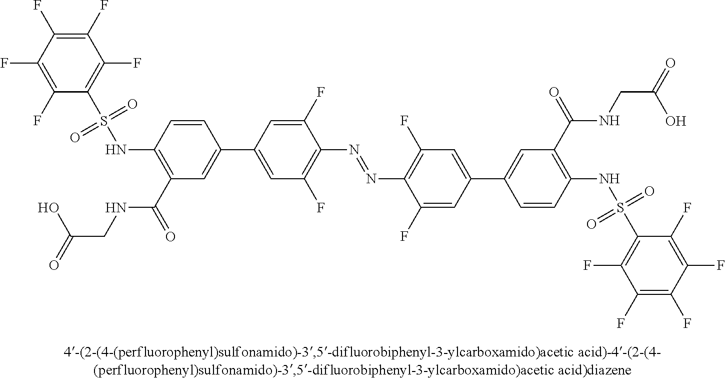 [SMILES: O=C(O)CNC(=O)c1cc(-c2cc(F)c(/N=N/c3c(F)cc(-c4ccc(NS(=O)(=O)c5c(F)c(F)c(F)c(F)c5F)c(C(=O)NCC(=O)O)c4)cc3F)c(F)c2)ccc1NS(=O)(=O)c1c(F)c(F)c(F)c(F)c1F]

In another preferred embodiment of the first aspect of the invention, the compound is 4′-(2-(4-(2-iodoacetamido)-3′,5′-dichlorobiphenyl-3-ylcarboxyamido)acetic acid)-4′-(2-(4-(2-iodoacetamido)-3′,5′-dichlorobiphenyl-3-ylcarboxyamido)acetic acid)diazene.

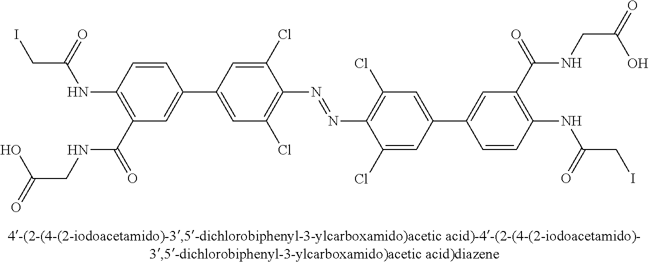 [SMILES: O=C(O)CNC(=O)c1cc(-c2cc(Cl)c(/N=N/c3c(Cl)cc(-c4ccc(NC(=O)CI)c(C(=O)NCC(=O)O)c4)cc3Cl)c(Cl)c2)ccc1NC(=O)CI]

In another preferred embodiment of the first aspect of the invention, the compound is 4′-(2-(4-(2-bromoacetamido)-3′,5′-dichlorobiphenyl-3-ylcarboxyamido)acetic acid)-4′-(2-(4-(2-bromoacetamido)-3′,5′-dichlorobiphenyl-3-ylcarboxyamido)acetic acid)diazene.

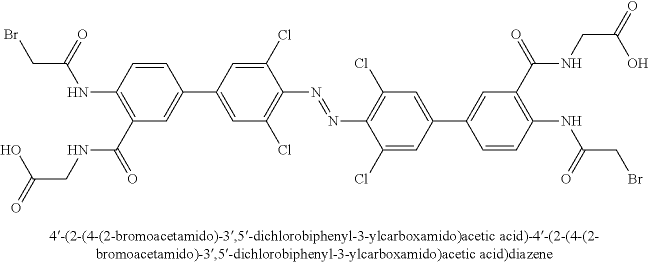 [SMILES: O=C(O)CNC(=O)c1cc(-c2cc(Cl)c(/N=N/c3c(Cl)cc(-c4ccc(NC(=O)CBr)c(C(=O)NCC(=O)O)c4)cc3Cl)c(Cl)c2)ccc1NC(=O)CBr]

In another preferred embodiment of the first aspect of the invention, the compound is 4′-(2-(4-(2-chloroacetamido)-3′,5′-dichlorobiphenyl-3-ylcarboxyamido)acetic acid)-4′-(2-(4-(2-chloroacetamido)-3′,5′-dichlorobiphenyl-3-ylcarboxyamido)acetic acid)diazene.

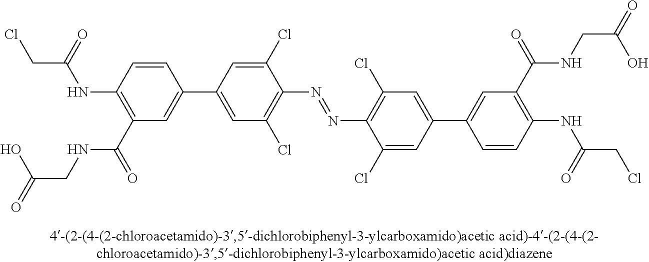 [SMILES: O=C(O)CNC(=O)c1cc(-c2cc(Cl)c(/N=N/c3c(Cl)cc(-c4ccc(NC(=O)CCl)c(C(=O)NCC(=O)O)c4)cc3Cl)c(Cl)c2)ccc1NC(=O)CCl]

In another preferred embodiment of the first aspect of the invention, the compound is 4′-(2-(4-(2,5-dioxo-2,5-dihydro-1H-pyrrol-1-yl)acetamido)-3′, 5′-dichlorobiphenyl-3-ylcarboxamido)acetic acid)-4′-(2-(4-(2,5-dioxo-2,5-dihydro-1H-pyrrol-1-yl)acetamido)-3′,5′-dichlorobiphenyl-3-ylcarboxamido)acetic acid)diazene.

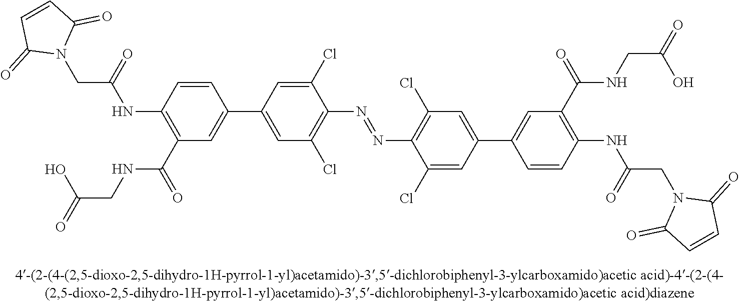 [SMILES: O=C(O)CNC(=O)c1cc(-c2cc(Cl)c(/N=N/c3c(Cl)cc(-c4ccc(NC(=O)CN5C(=O)C=CC5=O)c(C(=O)NCC(=O)O)c4)cc3Cl)c(Cl)c2)ccc1NC(=O)CN1C(=O)C=CC1=O]

In another preferred embodiment of the first aspect of the invention, the compound is 4′-(2-(4-(perfluorophenyl)sulfonamido)-3′,5′-dichlorobiphenyl-3-ylcarboxamido)acetic acid)-4′-(2-(4-(perfluorophenyl) sulfonamido)-3′,5′-dichlorobiphenyl-3-ylcarboxamido)acetic acid)diazene.

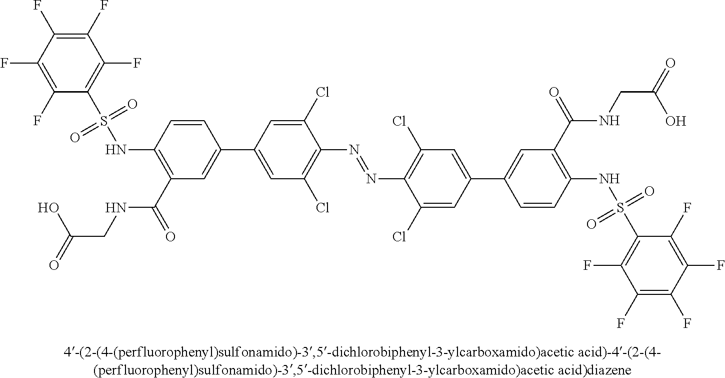 [SMILES: O=C(O)CNC(=O)c1cc(-c2cc(Cl)c(/N=N/c3c(Cl)cc(-c4ccc(NS(=O)(=O)c5c(F)c(F)c(F)c(F)c5F)c(C(=O)NCC(=O)O)c4)cc3Cl)c(Cl)c2)ccc1NS(=O)(=O)c1c(F)c(F)c(F)c(F)c1F]

In another preferred embodiment of the first aspect of the invention, the compound is 4′-(2-(4-(2-chloroacetamido)-3′,5′-dibromobiphenyl-3-ylcarboxyamido)acetic acid)-4′-(2-(4-(2-chloroacetamido)-3′,5′-dibromobiphenyl-3-ylcarboxyamido)acetic acid)diazene.

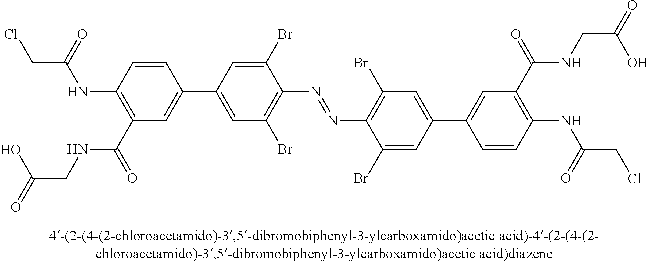 [SMILES: O=C(O)CNC(=O)c1cc(-c2cc(Br)c(/N=N/c3c(Br)cc(-c4ccc(NC(=O)CCl)c(C(=O)NCC(=O)O)c4)cc3Br)c(Br)c2)ccc1NC(=O)CCl]

In a preferred embodiment of the first aspect of the invention, the compound is more compact in the cis-state and more stretched in the trans-state, more preferably the distance between the flanking C₄-atoms of the azobiaryl differs by about 0.5 nm to about 50 nm between cis and trans, even more preferably about 1 nm to about 30 nm, most preferably about 1 nm to about 20 nm.

Cis- and trans-states were modeled via computational chemistry using an energy minimization algorithm (MM2 force field method available in ChemBio3D 19.0 from PerkinElmer Informatics, Inc.). The distance of atoms in the p-position of the second aryl was measured in each state using ChemDraw software (ChemDraw Professional Version 20.1.0.112, PerkinElmer Informatics, Inc.).

In one preferred embodiment of the first aspect of the invention, the configuration of the photoswitchable azobiaryl compound can be altered by irradiating with (a) particular wavelength(s) of light in a reversible manner, more preferably wherein the configuration is switched from a cis to a trans-state, or alternatively more preferably wherein the configuration is switched from a trans to a cis-state.

In one preferred embodiment of the preceding embodiment of the first aspect of the invention, the wavelength(s) of light are at least 400 nm, more preferably at most 750 nm, even more preferably at most 700 nm.

In a preferred embodiment of the first aspect of the invention, at least 80% of the compound is in the trans-state when exposed to wavelength(s) of light (a first wavelength λ1) from about 400 nm to 490 nm, and 80% is in the cis-state when exposed to wavelength(s) of light (a second wavelength λ2) from about 600 nm to 700 nm.

In another preferred embodiment of the first aspect of the invention, the compound is soluble at pH 8.0 in water from about 0.001 mM to about 2 mM at room temperature, more preferably more than 0.1 mM, most preferably more than 1 mM.

In a preferred embodiment of the first aspect of the invention, the thermal half-life of the cis state at room temperature in water at pH 8.0 is from about 1 minute to about 72 h, more preferably more than 1 h, most preferably more than 12 h. The ratio of cis to trans was determined by HPLC analyses.

The photoswitchable azobiaryl compound may be one that changes from a first isomeric state to a second isomeric state upon exposure to light of different wavelengths, or upon a change in exposure from dark to light, or from light to dark. For example, in some embodiments, the photoswitchable azobiaryl compound may be in a first isomeric state when exposed to light of a first wavelength λ1, and may be in a second isomeric state when exposed to light of a second wavelength λ2.

The first wavelength and the second wavelength can differ from one another by from about 1 nm to about 1000 nm or more, preferably from about 50 nm to about 500 nm, more preferably from about 80 nm to about 400 nm, particularly preferably from about 100 nm to about 300 nm. In a preferred embodiment, the first wavelength and the second wavelength differ from one another by at least 100 nm. In another preferred embodiment, the first wavelength and the second wavelength differ from one another by at most 300 nm.

In other embodiments, the photoswitchable azobiaryl compound is in a first isomeric state when exposed to light of a wavelength, and is in a second isomeric state in the absence of light (e.g., in the absence of light, the photoswitchable azobiaryl compound undergoes thermal relaxation into the second isomeric state). In these embodiments, the first isomeric state is induced by exposure to light of wavelength λ1, and the second isomeric state is induced by not exposing the photoswitchable azobiaryl compound to light, e.g., keeping the photoswitchable azobiaryl compound in darkness.

In other embodiments, the photoswitchable azobiaryl compound is in a first isomeric state in the absence of light, e.g., when the photoswitchable azobiaryl compound is in the dark; and the photoswitchable azobiaryl compound is in a second isomeric state when exposed to light of a wavelength λ2.

In other embodiments, the photoswitchable azobiaryl compound is in a first isomeric state when exposed to light of a first wavelength, and the photoswitchable azobiaryl compound is in a second isomeric state when exposed to light of second wavelength. For example, in some embodiments, the photoswitchable azobiaryl compound is in a trans-configuration in the absence of light, or when exposed to light of a first wavelength; and the trans-configuration is in a cis-configuration when exposed to light, or when exposed to light of a second wavelength that is different from the first wavelength.

As another example, in some embodiments, the photoswitchable azobiaryl compound is in a cis-configuration in the absence of light, or when exposed to light of a first wavelength; and the photoswitchable azobiaryl compound is in a trans-configuration when exposed to light, or when exposed to light of a second wavelength that is different from the first wavelength.

The wavelength of light that effects a change from a first isomeric state to a second isomeric state ranges generally from 1 nm to about 2000 nm. “Light,” as used herein, refers to electromagnetic radiation, including, but not limited to, ultraviolet light, visible light, infrared, and microwave.

In some embodiments, the intensity of the light can vary from about 1 W/m² to about 50 W/m², e.g., from about 1 W/m² to about 5 W/m², from about 5 W/m² to about 10 W/m², from about 10 W/m², from about 10 W/m² to about 15 W/m², from about 15 W/m² to about 20 W/m², from about 20 W/m² to about 30 W/m², from about 30 W/m² to about 40 W/m², or from about 40 W/m² to about 50 W/m².

In other embodiments, the intensity of the light can vary from about 1 μW/cm² to about 100 μW/cm², e.g., from about 1 μW/cm² to about 5 μW/cm², from about 5 μW/cm² to about 10 μW/cm², from about 10 μW/cm² to about 20 μW/cm², from about 20 μW/cm² to about 25 μW/cm², from about 25 μW/cm² to about 50 μW/cm², from about 50 μW/cm² to about 75 μW/cm², or from about 75 μW/cm² to about 100 μW/cm².

In further embodiments, the intensity of light varies from about 1 μW/mm² to about 1 W/mm², e.g., from about 1 μW/mm² to about 50 μW/mm². from about 50 μW/mm² to about 100 μW/mm². from about 100 ρW/mm². to about 500 μW/mm². from about 500 μW/mm² to about 1 mW/mm², from about 1 mW/mm² to about 250 mW/mm². from about 250 mW/mm² to about 500 mW/mm², or from about 500 mW/mm² to about 1 W/mm².

Also, in a second aspect of the present invention, a photoswitchable affinity ligand is provided comprising an affinity ligand in stable association with the photoswitchable azobiaryl compound according to the first aspect of the invention as described hereinabove.

In the context of the present invention, an affinity ligand may preferably be defined as a chemical entity which is able to specifically and selectively interact with and bind to a target molecule.

In a preferred embodiment of the second aspect of the invention, the affinity ligand is selected from the group consisting of a peptide, an oligopeptide, a polypeptide, a protein, an antibody or an antigen-binding fragment thereof, an immunoglobulin or a fragment thereof, an enzyme, a hormone, a cytokine, a complex, an oligonucleotide, a polynucleotide, a nucleic acid, an aptamer, a carbohydrate, a liposome, a nanoparticle, a cell, a biomacromolecule, a biomolecule or a small molecule.

In one preferred embodiment of the second aspect of the invention, the affinity ligand is selected from the group comprising immunoglobulin (Ig)-binding proteins, more preferably selected from the group comprising protein A, protein G and protein L or variants thereof with the ability to specifically bind to immunoglobulins.

Protein A, protein G or protein L affinity chromatography is often used in commercial purification processes for pharmaceutical grade monoclonal antibodies. Protein A is a bacterial cell wall protein that binds to mammalian antibodies, primarily through hydrophobic interactions along with hydrogen bonding and two salt bridges with the antibodies' Fc regions.

Thus, in the context of chromatographic purification, protein A resins allow for the affinity-based retention of antibodies on a chromatographic support, while unwanted components in a clarified harvest flow past the support and can be discarded. The retained antibodies can then be eluted from the chromatographic support by disrupting the antibody-protein A interaction. Common elution conditions by means of low pH take on a positive charge on highly conserved (de) ionizable amino acid residues that face each other on the protein A-Fc region, thus repelling each other and decreasing the hydrophobic contact area between the two molecules. However, the common elution based on acidic elution conditions and low PH levels may lead to chemical modification or denaturation of the antibody and/or affinity matrix and, thus, affect functionality.

Modifying wild-type protein A (SEQ ID NO. 1, UniProtKB entry P38507), wild-type protein G (SEQ ID NO. 2, UniProtKB entry P19909) or wild-type protein L (SEQ ID NO. 3, UniProtKB entry Q51918) by stable association of a photoswitchable azobiaryl compound to optically control their binding activity and applying them to generate a photoswitchable affinity matrix would diminish the disadvantages of this conventional purification technique.

When coupling an affinity ligand to a solid support, multi-point or single-point attachment may be used. Multi-point coupling can impede the flexibility of the 3-dimensional structure and thus the structural rearrangement by altering the photoswitchable azobiaryl compound. The advantage of single-point attachment is that it allows for greater flexibility of the 3-dimensional structure of the affinity ligand, potentially preserving its binding properties. This can result in higher specificity and activity compared to multi-point attachment. Single-point attachment can be achieved by covalently linking the affinity ligand to the solid support through a reactive functional group on the ligand, such as a primary amine or a thiol, using standard coupling reactions. Using primary amines for covalent immobilization, single-point attachment can be achieved by protein variants that carry none, one or a defined set of lysine residues at a specific position.

In one preferred embodiment, the present invention relates to the affinity ligand having a single (N-terminal) or, by the introduction of lysine residues, a defined set of amino groups for site-specific single or multipoint attachments.

In one preferred embodiment, the present invention relates to the affinity ligand being a variant of an immunoglobulin (Ig)-binding protein, e.g., protein A, protein G or protein L. The variant may comprise the Ig-binding protein having at least one residue substituted with a cysteine residue. The at least one substitution may provide a conjugation site for the stable association with a photoswitchable azobiaryl compound bearing one or two reactive sites. The modification of an Ig-binding protein with a photoswitchable azobiaryl compound may provide optical modulation of the binding activity.

Protein A comprises five homologous Ig-binding domains that each fold into a three-helix bundle. Each of these five domains is able to bind antibodies from many mammalian species, most notably those belonging to the class of immunoglobulin G (IgG). For affinity purification purposes often a recombinant fragment of protein A is used. This fragment comprises or consists of domain B of protein A. More specifically, protein A binds to the Fc region within the heavy chain of most immunoglobulins, and also to the Fab region, especially in the case of the human VH3 family.

In one preferred embodiment of the second aspect of the invention, the affinity ligand comprises the B domain of protein A (SEQ ID NO. 4), preferably comprising residues 215-268 of SEQ ID NO. 1 (UniProtKB entry P38507, amino acid numbering is based on the full length sequence), optionally substituted with one or two cysteine residues, or comprises a protein domain having at least 80% sequence identity to SEQ ID NO. 4, more preferably wherein the affinity ligand comprises a protein domain having the amino acid sequence of SEQ ID NO. 6 or SEQ ID NO. 7, even more preferably wherein the affinity ligand comprises a protein domain having the amino acid sequence of SEQ ID NO. 7.

In another preferred embodiment of the second aspect of the invention, the affinity ligand comprises a lysine-deficient B domain of protein A (SEQ ID NO. 5), optionally substituted with one or two cysteine residues, or comprises a protein domain having at least 80% sequence identity to SEQ ID NO. 5, more preferably wherein the affinity ligand comprises a protein domain having the amino acid sequence of SEQ ID NO. 8, SEQ ID NO. 9, SEQ ID NO. 10 or SEQ ID NO. 11, even more preferably wherein the affinity ligand comprises a protein domain having the amino acid sequence of SEQ ID NO. 11.

In one preferred embodiment, the affinity ligand may comprise the wild-type (SEQ ID NO. 4) or lysine-deficient B domain of protein A (SEQ ID NO. 5). According to another preferred embodiment, the wild-type or lysine-deficient B domain of protein A may be mutated to carry one or more amino acid substitutions or mutations of residues selected from the group comprising Lys215, Phe216, Asn217, Lys218, Glu219, Asn234, Glu236, Gly240, Phe241, Lys246, Asp247, Asp248, Ser250, Ala253, Asn254, Lys260, Lys261, Ala265 and Ala267 (shown in bold in SEQ ID NO. 4 represented below) may be used.

In one preferred embodiment, the single substitution may preferably be selected from Asn217Cys (i.e., Asn in position 217 substituted with or mutated to cysteine), Glu219Cys, Glu236Cys, Gly240Cys, Asp247Cys, Asp248Cys, Ser250Cys, Ala253Cys, Asn254 Cys, Ala265Cys or Ala267Cys. The double substitutions may preferably be selected from Asn217Cys/Asp248Cys, Glu219Cys/Asp248Cys, Glu219-Cys/Ala267Cys Glu236Cys/Asp247Cys, Gly240Cys/Ala265Cys or Gly240Cys/Asn254Cys.

In another preferred embodiment, residues 215-216 in SEQ ID NO. 4 or SEQ ID NO. 5 may preferably be replaced by Lys213-Ala214-Cys215-Gly216 (as in SEQ IDs NO. 6 to 9), and may preferably each have one additional substitution selected from Asp247Cys (SEQ ID NO. 7), Asp248Cys, Ser250Cys (SEQ ID NO. 9) or Ala253Cys.

In yet another embodiment, residues 215-216 in SEQ ID NO. 5 may preferably be replaced by Lys209-Gly210-Gly211-Gly212-Gly213-Ala214-Ser215-Phe216 to provide a linker between the solid support and the affinity ligand, and may preferably have double substitutions selected from Asn217Cys/Asp248Cys (SEQ ID NO. 10), Glu219Cys/Asp248Cys (SEQ ID NO. 11), Glu219Cys/Ala267Cys Glu236Cys/Asp247Cys, Gly240Cys/Ala265Cys or Gly240Cys/Asn254Cys.

Particular sequences of the Ig-binding affinity ligand may preferably be extended at the N-terminus with a Met residue and at the C-terminus by Ser-Ala-His-His-His-His-His-His.

| | |
|---|---|
| | 215 216 230 240 250 260 |
| | \| \| \| \| \| \| |
| SEQ ID NO. 4 | MK FNKEQQNAFYEILHLPNLNEEQRNGFIQSLKDDPSQSANLLAEAKKLNDAQAP |
| SEQ ID NO. 5 | MS FNMEQQNAFYEILHLPNLNEEQRNGFIQSLRDDPSQSANLLAEAQELNDAQAP |
| SEQ ID NO. 6 | MK ACGNKEQQNAFYEILHLPNLNEEQRNGFIQSLKDDPSQSANLLAEAKKLNDAQAP |
| SEQ ID NO. 7 | MK ACGNKEQQNAFYEILHLPNLNEEQRNGFIQSLKDDPCQSANLLAEAKKLNDAQAP |
| SEQ ID NO. 8 | MK ACGNMEQQNAFYEILHLPNLNEEQRNGFIQSLRDDPSQSANLLAEAQELNDAQAP |
| SEQ ID NO. 9 | MK ACGNMEQQNAFYEILHLPNLNEEQRNGFIQSLRDDPCQSANLLAEAQELNDAQAP |
| SEQ ID NO. 10 | MKGGGGASFCMEQQNAFYEILHLPNLNEEQRNGFIQSLRDCPSQSANLLAEAQELNDAQAP |
| SEQ ID NO. 11 | MKGGGGASFNMCQQNAFYEILHLPNLNEEQRNGFIQSLRDCPSQSANLLAEAQELNDAQAP |

Protein G is also an immunoglobulin-binding protein, found in group C and G Streptococci. Besides an albumin-binding region, it consists of three Ig-binding domains with specific binding affinity for the antibody Fc and Fab region.

In one embodiment of the second aspect of the present invention, the affinity ligand of the present invention may preferably comprise at least one of the three homologous domains of protein G in SEQ ID NO. 2 (UniProtKB entry P19909; amino acid numbering is based on the full length sequence), defined as C1 (residues 303-357; also called B1 elsewhere), C2 (residues 373-427) and C3 (residues 443-497; also denoted B2).

In one embodiment of the second aspect, the affinity ligand of the present invention may preferably comprise a domain of streptococcal protein G, more preferably having at least one residue of the C1, C2 or C3 domain of protein G substituted with a cysteine residue.

In another preferred embodiment of the second aspect of the invention, the affinity ligand comprises at least one of the three homologous domains of protein G, defined as C1 (SEQ ID NO. 12), C2 (SEQ ID NO. 13) and C3 (SEQ ID NO. 14) optionally carrying two substitutions of wild-type residues with cysteines or comprises a protein domain having at least 80% sequence identity to SEQ ID NO. 12, SEQ ID NO. 13 or SEQ ID NO. 14.

In another preferred embodiment of the second aspect of the present invention, the affinity ligand of the present invention comprises the lysine-deficient C1 domain of Protein G with lysine residues substituted to any other amino acid except lysine and a Asn338Tyr substitution to increase the stability against alkaline hydrolysis (SEQ ID NO. 15).

Individual domains may be extended at the N-terminus with the sequence Met-Lys (SEQ IDs NO: 12, 13 and 14) or Met-Ser (SEQ ID NO: 15) and carry single or double substitutions, such as mutations, of corresponding residues in either domain. More specifically, the protein G domain C1 used herein preferably comprises substitutions of one or more of residues Thr303, Lys305, Lys314, Lys311, Glu316, Lys314, Glu320, Val322, Lys329, Lys332, Asp337, Asn338, Thr345, Asp348, Lys351, Thr350 and Thr356 (shown in bold in SEQ ID NO. 12 represented below). Accordingly, one or more equivalent substitutions at corresponding homologous positions in domains C2 and C3 may preferably be comprised. These substitutions, either individually or in all conceivable combinations, are also considered preferred.

The single substitution within domain C1 may preferably be selected from Thr303Cys (i.e., Thr at position 303 substituted with or mutated to cysteine), Lys305Cys, Lys311Cys, Lys314Cys, Glu316Cys, Glu320Cys, Val322Cys, Lys329Cys, Lys332Cys, Asp337Cys, Asn338Cys, Thr345Cys, Asp348Cys, Thr350Cys, Lys351Cys, and Thr356Cys.

Double substitutions in domain C1 may preferably be selected from Thr303Cys/Thr356Cys, Lys305Cys/Lys314Cys, Lys305Cys/Thr445Cys, Lys305Cys/Thr356Cys, Lys311Cys/Thr350Cys, Lys314Cys/Thr350Cys, Lys314Cys/Thr356Cys, Glu316Cys/Thr345Cys, Glu320Cys/Asp337Cys, Glu320Cys/Asn338Cys, Glu320Cys/Thr350Cys, Val322Cys/Asp348Cys or Thr350Cys/Thr356Cys.

Domains C2 and C3 may preferably comprise single and double Cys mutations at corresponding, homologous positions. Individual sequences may be extended at the C-terminus with the sequence Ser-Ala-His-His-His-His-His-His.

| | |
|---|---|
| | 303 310 320 330 340 350 |
| | \| \| \| \| \| \| |
| SEQ ID NO. 12 | MKTYKLILNGKTLKGETTTEAVDAATAEKVFKQYANDNGVDGEWTYDDATKTFTVTE |
| SEQ ID NO. 15 | MSTYRLILNGVTLSGETTTEAVDAATAERVFRQYANDYGVDGEWTYDDATRTFTVTE |

Protein L is a bacterial surface protein and is important for pathogenic immune evasion. The full-length protein L of Finegoldia magna comprises an N-terminal region with an affinity for a diverse set of immunoglobulins, an additional region with albumin binding domains and cell wall-spanning as well as membrane anchor domains. The four homologous Ig-binding domains found in Finegoldia magna strain 3316 have a specific affinity for immunoglobulin light chains.

In terms of antibody purification, interaction with the (kappa) light chain is an advantage over protein A or G, since additional antibody classes like IgA, IgM, IgE or IgD can be bound by protein L. Furthermore, light chain specificity of an Ig-binding affinity ligand enables the purification of antibody fragments, like single-chain variable fragments (scFv) and Fab fragments or fusions thereof.

In one preferred embodiment of the second aspect of the invention, the affinity ligand comprises one C-domain of protein L, having a least one residue substituted with a cysteine residue or a protein domain having at least 80% sequence identity thereto, more preferably wherein the C-domain of protein L is the C2 domain of protein L (SEQ ID NO. 16), defined as residues 326-389 corresponding to UniProtKB entry Q51918 (SEQ ID NO. 3), or a protein domain having at least 80% sequence identity thereto.

In another preferred embodiment of the second aspect of the present invention, the affinity ligand of the present invention comprises the C2 domain of Protein L with lysine residues substituted to any other amino acid except lysine (SEQ ID NO. 17) to provide site-specific immobilization via amino reactive chemistry.

In one preferred embodiment, the Ig-binding affinity ligand of the present invention may comprise the C2 domain of protein L (SEQ ID NO. 16). More specifically, the protein L domain C2 used herein preferably comprises substitutions of one or more of residues Lys326, Lys332, Ile336, Lys 341, Thr342, Lys348 Glu353, Lys357, Lys367, Glu377, Asp378, Thr382 and Lys386 (shown in bold in SEQ ID NO. 16 represented below).

The single substitution may be selected from Ile336Cys (i.e. Ile in position 336 substituted with or mutated to cysteine), Thr342Cys, Glu353Cys, Lys367Cys, Glu377Cys or Asp378Cys. The double substitutions may be selected from Ile336Cys/Asp378Cys, Thr342Cys/Glu377Cys or Thr342Cys/Asp378Cys. Particular sequences of the Ig-binding affinity ligand may be extended at the N-terminus with a Met residue and at the C-terminus by the sequence His-His-His-His-His-His.

| | |
|---|---|
| | 326 330 340 350 360 370 380 389 |
| | \| \| \| \| \| \| \| \| |
| SEQ ID NO. 16 | MKEEVTIKVNLIFADGKTQTAEFKGTFEEATAKAYAYADLLAKENGEYTADLEDGGNTINIKFAG |
| SEQ ID NO. 17 | MAEEVTIRVNLIFADGSTQTAEFRGTFEEATAEAYAYADLLARENGEYTADLEDGGNTINIRFAG |

In the following, the amino acid sequences of specifically preferred individual affinity ligand protein domains are given in the common one letter order:

(B domain of protein A)
SEQ ID NO: 4
MKFNKEQQNAFYEILHLPNLNEEQRNGFIQSLKDDPSQSANLLAEAKKLNDAQAP
(lysine-deficient B domain of protein A)
SEQ ID NO: 5
MSFNMEQQNAFYEILHLPNLNEEQRNGFIQSLRDDPSQSANLLAEAQELNDAQAP
(affinity ligand protein domain)
SEQ ID NO: 6
MKACGNKEQQNAFYEILHLPNLNEEQRNGFIQSLKDDPSQSANLLAEAKKLNDAQAP
(affinity ligand protein domain)
SEQ ID NO: 7
MKACGNKEQQNAFYEILHLPNLNEEQRNGFIQSLKDDPCQSANLLAEAKKLNDAQAP
(affinity ligand protein domain)
SEQ ID NO: 8
MKACGNMEQQNAFYEILHLPNLNEEQRNGFIQSLRDDPSQSANLLAEAQELNDAQAP
(affinity ligand protein domain)
SEQ ID NO: 9
MKACGNMEQQNAFYEILHLPNLNEEQRNGFIQSLRDDPCQSANLLAEAQELNDAQAP
(affinity ligand protein domain)
SEQ ID NO: 10
MKGGGGASFCMEQQNAFYEILHLPNLNEEQRNGFIQSLRDCPSQSANLLAEAQELNDAQAP
(affinity ligand protein domain)
SEQ ID NO: 11
MKGGGGASFNMCQQNAFYEILHLPNLNEEQRNGFIQSLRDCPSQSANLLAEAQELNDAQAP
(protein G domain C1, residues 303-357 of SEQ ID NO: 2)
SEQ ID NO: 12
MKTYKLILNGKTLKGETTTEAVDAATAEKVFKQYANDNGVDGEWTYDDATKTFTVTE
(protein G domain C2, residues 373-427 of SEQ ID NO: 2)
SEQ ID NO: 13
MKTYKLVINGKTLKGETTTEAVDAATAEKVFKQYANDNGVDGEWTYDDATKTFTVTE
(protein G domain C3, residues 443-497 of SEQ ID NO: 2)
SEQ ID NO: 14
MKTYKLVINGKTLKGETTTKAVDAETAEKAFKQYANDNGVDGVWTYDDATKTFTVTE
(C1 domain of protein G)
SEQ ID NO: 15
MSTYRLILNGVTLSGETTTEAVDAATAERVFRQYANDYGVDGEWTYDDATRTFTVTE
(C2 domain of protein L)
SEQ ID NO: 16
MKEEVTIKVNLIFADGKTQTAEFKGTFEEATAKAYAYADLLAKENGEYTADLEDGGNTINIKFAG
(lysine-deficient C2-domain of protein L)
SEQ ID NO: 17
MAEEVTIRVNLIFADGSTQTAEFRGTFEEATAEAYAYADLLARENGEYTADLEDGGNTINIRFAG
(variant SpG#1 of C1 domain of protein G)
SEQ ID NO: 18
MATKASKGGGGTTYRLILNGVTLSGETTTEACDAATAERVFRQYANDYGVDGEWTYDCATRTFTVTE
GEDEDEDED
(variant PpL#1 of C2 domain of protein L)
SEQ ID NO: 19
MATKASKGGGGASEEVTIRVNLIFADGSTQCAEFRGTFEEATAEAYAYADLLARENGEYTACLEDGG
NTINIRFAGEDEDEDED

In one preferred embodiment of the second aspect of the invention, the protein L domain C2 used herein preferably comprises one or more of the amino acid substitutions selected from the list consisting of Thr344Cys, Glu346Cys, and Asp375Cys. According to a preferred embodiment, a single substitution may be selected from Thr344Cys, Glu346Cys, and Asp375Cys. Preferably, double substitutions may be selected from Glu346Cys/Asp378Cys, and Thr344Cys/Asp375Cys.

In one preferred embodiment of the second aspect of the invention, the photoswitchable azobiaryl compound is stably associated with two conjugation sites within the affinity ligand in a bi- or difunctional manner.

In another preferred embodiment of the second aspect of the invention, exposure to light of a specific wavelength induces a conformational switch causing a change of specific affinity of the photoswitchable affinity ligand to the target molecule.

As discussed above, the present invention provides a photoswitchable affinity ligand. Said ligand comprises at least one reactive moiety for stable association of the photoswitchable azobiaryl compound according to the present invention. Said affinity ligand can further comprise additional moieties for stable association to a solid support.

Modification of an affinity ligand can preferably be done with mono-, bi- or difunctional photoswitchable azobiaryl compounds with regard to the ligand-reactive moiety. Optical modulation of the ligand affinity using monofunctional photoswitches relies on steric effects (e.g., interference with ligand binding), whereas modulation using bifunctional photoswitches usually is intended to modulate the conformation of the ligand and thus its specific affinity.

One preferred embodiment of the present invention relates to an affinity ligand stably associated with bi- or difunctional photoswitchable azobiaryl compound in the cis-state. This modification of the affinity ligand is preferably achieved by introducing two conjugation sites (e.g., thiol groups of cysteine side chains), which can be cross-linked by the bifunctional photoswitchable azobiaryl compound in a highly specific manner. These photoswitchable affinity ligands can be induced to change their conformation and/or their affinity in a reversible manner by illumination with light.

The photoswitchable azobiaryl compound attached in the more compact cis-state to the affinity ligand at two specific positions (conjugation sites) is expected to preserve the native conformation of the affinity ligand upon exposure to light of a first wavelength. Photoisomerization from the more compact cis to the stretched trans-state upon exposure to light of a second wavelength preferably distorts the affinity ligand and thus disfavors binding of the target molecule.

Another preferred embodiment relates to the modification of an affinity ligand with bi- or difunctional photoswitches in the trans-state. A photoswitchable azobiaryl compound attached at two specific positions (conjugation sites) in the stretched trans-state is expected to preserve the native conformation of the affinity ligand upon exposure to light of a first wavelength. Photoisomerization from the trans to the more compact cis-state upon exposure to light of a second wavelength preferably distorts the affinity ligand and thus disfavors binding of the target molecule.

Yet another preferred embodiment relates to the modification of an affinity ligand with monofunctional photoswitches in the cis-state. A photoswitchable azobiaryl compound attached at one specific position (conjugation site) preferably allows binding of the target molecule in the more compact cis-state upon exposure to light of a first wavelength but sterically overlaps in the stretched trans-state upon exposure to light of a second wavelength and thus disfavor binding.

A further preferred embodiment relates to the modification of an affinity ligand with monofunctional photoswitch in the trans-state. A photoswitchable azobiaryl compound attached at one specific position (conjugation site) preferably allows binding of the target molecule in the stretched trans-state upon exposure to light of a first wavelength but sterically overlaps in the more compact cis-state upon exposure to light of a second wavelength and thus disfavors binding.

In one preferred embodiment, the photoswitchable affinity ligand of the present invention may be modified by recombinant means to include a spacer or linker sequence as either an N- or C-terminal extension and thereby form a fusion ligand-linker product, which may confer improved immobilization to a solid support. In this regard, as one of skill in the art will appreciate, the linker or spacer may also be chemically synthesized and covalently bound to the selected photoswitchable affinity ligand using well-established methodologies.

Alternatively, a fusion product comprising the linker and photoswitchable affinity ligand of the present invention may be made using recombinant techniques. In this regard, the linker may be generally bound or fused to a terminus of the photoswitchable affinity ligand such that the function of the photoswitchable affinity ligand may be substantially retained. Thus, depending on the protein, the linker may be bound to the N-terminus or the C-terminus of the photoswitchable affinity ligand. In certain instances, the linker may be bound to both termini, or may additionally be bound to an amino acid residue which is not a terminal residue.

In another alternative, the linker may be bound to a solid support by chemical means or through the use of an enzyme prior to the coupling of the photoswitchable affinity ligand. The linker may be suitable to immobilize the photoswitchable affinity ligand of the present invention onto a solid support while substantially retaining the function of the photoswitchable affinity ligand, i.e., retaining at least about 50% of immunoglobulin binding, in its immobilized state compared to the unbound state.

A photoswitchable affinity ligand according to the present invention may be prepared by selecting at least one conjugation site in an affinity ligand for stable association of a photoswitchable azobiaryl compound. Said conjugation site, such as a cysteine residue, may be added to the affinity ligand by substitution or insertion, if not already present. The conjugation site must be amenable to conjugation of an additional functional moiety described herein as ligand-reactive moiety or R³/R^{3′} as shown in Formula (I).

Photoswitches can be covalently associated to the selected conjugation sites through an assortment of different conjugation chemistries described here and known in the art. For example, a photoswitchable azobiaryl compound carrying reactive iodoacetyl groups targeting two accessible cysteine thiols on a polypeptide is one embodiment, but numerous conjugation or coupling chemistries targeting the side chains of either canonical or non-canonical amino acids, can be employed in accordance with the present invention.

The selection of the placement of the conjugation sites in the affinity ligand is another important facet. Any of the exposed amino acid residues on the affinity ligand surface, can be a potentially useful conjugation site and may be mutated to cysteine or some other reactive amino acid for covalent association, if not already present at the selected conjugation site of the affinity ligand sequence. Steric hindrance between cross-linked photoswitches and the affinity ligand binding cleft should be avoided so that specific binding is preserved.

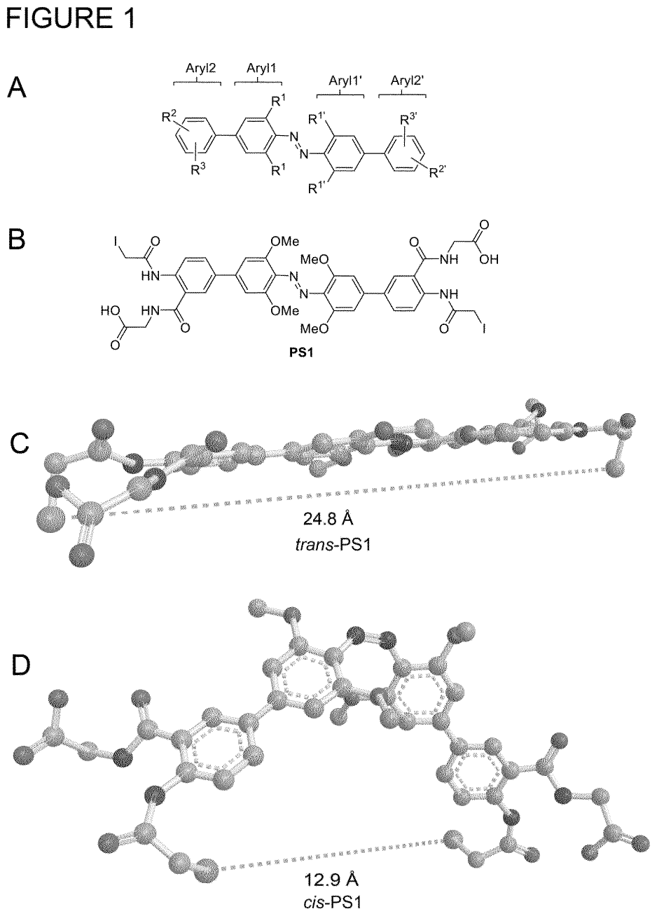

In one preferred embodiment, a water-soluble derivative of a photoswitch in accordance with the present invention is selected to stabilize the native conformation of an affinity ligand (e.g., protein A) in the more compact cis-state, and to distorted it in the stretched trans-state. This criterion determines the distance between conjugation sites in the affinity ligand that are being cross-linked via a bifunctional (thiol-reactive) photoswitch. In this regard, an ideal distance between conjugation sites can be assumed from the length of the photoswitchable azobiarylcompound in its cis-state of about 11-16 Å (FIG. 1D). Using a protein structure (model) of the affinity ligand, suitable residues (cysteine pair; Sy side chain atoms) that satisfy this distance criterion can be determined.

In a further preferred embodiment, a water-soluble derivative of a photoswitch in accordance with the present invention was selected to stabilize the native conformation of an affinity ligand in the trans-state, and to destabilize it in the more compact cis-state. This criterion determines the distance between conjugation sites that are being cross-linked via a bifunctional (thiol-reactive) photoswitches.

To that end, an ideal distance between conjugation sites can be assumed from the length of the photoswitchable azobiarylcompound in its trans-state of about 20-26 Å (FIG. 1C). Using a protein structure (model) of the affinity ligand, suitable residues (cysteine pair; Sy side chain atoms) that satisfy this distance criterion can be determined.

According to the third aspect of the present invention, a photoswitchable affinity matrix is provided comprising a solid support, and a photoswitchable azobiaryl compound according to the first aspect of the invention in stable association with an affinity ligand, wherein the photoswitchable azobiaryl compound and the affinity ligand are in stable association with the solid support, or a photoswitchable affinity matrix comprising a photoswitchable affinity ligand according to the second aspect of the invention in stable association with the solid support.

The photoswitchable matrix according to the present invention is preferably provided for optical controlled affinity separation. The matrix may comprise photoswitchable affinity ligands that may be made of any of the affinity ligands and photoswitchable azobiaryl compounds of the present invention coupled to a solid support. An affinity matrix according to embodiments of the present invention exhibits the isolation of a target from an aqueous mixture by the control of light.

The affinity ligand could preferably be coupled (covalently or non-covalently) onto a solid support before or after it is functionalized via stable association of a photoswitchable azobiaryl compound. In one preferred embodiment, coupling of the photoswitchable affinity ligand to the solid support is mediated between a moiety of the affinity ligand and the solid support. In another preferred embodiment, coupling of the photoswitchable affinity ligand to the solid support is mediated between the photoswitchable azobiaryl compound moiety and the solid support. In yet another preferred embodiment, both of the affinity ligand moiety and the photoswitchable azobiaryl compound moiety contribute to the coupling of the photoswitchable affinity ligand to the solid support. Preferably, said coupling comprises covalent bonds.

The (photoswitchable) affinity ligand of the present invention may be attached to the solid support via conventional coupling techniques utilizing, e.g. amino and/or carboxy groups present in the ligand or any other functional group of the affinity ligand and/or the photoswitchable azobiaryl compound. The use of epoxide-, CNBr-, N-hydroxysuccinimidyl ester-activated solid supports and solid supports for copper catalyzed click chemistry are well-known immobilization procedures.

Between the support and the ligand, a spacer or linker may be introduced to facilitate the chemical coupling of the affinity ligand to the support, which will improve the availability of the photoswitchable affinity ligand. Alternatively, the photoswitchable affinity ligand may be attached to the support by non-covalent association, such as physical or biospecific adsorption.

In one embodiment, the (photoswitchable) affinity ligand of the present invention may be coupled to the support via primary amines (e.g. lysine side chains or N-terminus). Methods for performing such attachment is well-known in this field and easily performed by the person of ordinary skill in this field using standard techniques and equipment.

Suitable solid supports are preferably selected from the group comprising synthetic polymers (e.g., polysulfone (PSF), polyethersulfone (PES), polyacrilonitrile (PAN), polyamide (PA), polyethylene and polypropylene (PE and PP), polymethyl methacrylate (PMMA), polyglycidyl methacrylate (PGMA), Polysterene (PS)), non-synthetic polymers, such as polysaccharide (e.g., dextran, starch, cellulose, pullulan, or agarose), inorganic support (e.g., silica or zirconium oxide, magnetic particles) and any mixed composite solid support derived from mentioned or any surfaces having the chemistry to allow covalent association (chemical coupling) of an affinity ligand and/or a photoswitch.

Examples for the material of the solid support are based on polymers having a surface chemistry for covalent association, such as but not limited to polymers having hydroxyl groups (—OH), carboxyl groups (—COOH), amino groups (—NH₂, possibly in substituted form), epoxide groups, azide or alkyne groups for click chemistry.

In one preferred embodiment, the polymer is a synthetic polymer (e.g., polyethersulfone). Such a synthetic polymer may be a commercially available product.

In another preferred embodiment, the polymer is a polysaccharide (e.g., dextran, starch, cellulose, pullulan, or agarose). Such a polysaccharide may be a commercially available product.

In another preferred embodiment, the solid support is a magnetic particle. Such a magnetic particle may be a commercially available product.

The solid support may preferably be in the shape of particles. The particles may be porous or nonporous. The solid support in the shape of particles may be used as a packed bed, or may be used in a suspended form. The suspended form may be an expanded bed or a pure suspension, in which the particles can move freely. When using a packed bed, or an expanded bed, separation processes used in known affinity chromatographic methods may be used. When using a pure suspension, a batch method may be used.

The solid support in the shape of particles according to this embodiment preferably may have a particle size (diameter) of about 1 nm to about 500 micrometers, and more preferably about 100 nm to about 100 micrometers. Particle size can be determined by light-scattering, preferably using suitable particle size analyzers manufactured by Malvern Panalytical.

In yet another embodiment, the solid support may be in another form such as a monolith, a chip, a microtiter plate, capillaries, or a membrane.

In a preferred embodiment, the solid support may be in the shape of membranes. According to a preferred embodiment of the present invention, porous membranes with a large inner surface area are used. Porous membranes are preferred that have a BET surface area between 2 and 300 m² per cm³, and those membranes with a BET Surface area between 8 and 30 m² per cm³ are even more preferred. The BET method for determining the surface area of porous membrane structures, which is based on the measurement of nitrogen adsorption, is described by K. Kaneko (Kaneko, K. (1994). Determination of pore size and pore size distribution: 1. Adsorbents and catalysts. Journal of membrane science, 96 (1-2), 59-89).

There are no restrictions with respect to the material of the membranes according to the invention. Membranes can be used that are made of inorganic materials such as glass, ceramics, SiO₂, carbon, or metal, or of organic polymers or blends thereof. The polymers can be hydrophilic and/or hydrophobic in nature.

They can be selected from the group of cellulosic polymers such as cellulose or regenerated cellulose, modified cellulose such as cellulose esters, cellulose ethers, amine-modified celluloses, or blends of cellulosic polymers, from the group of synthetic polymer such as polyacrylonitrile and corresponding copolymers, polymers containing polyurethane, polyarylsulfones and polyarylethersulfones such as poly sulfone or polyethersulfone, polyvinylidene fluoride, polyacrylamide, polytetrafluoroethylene, water-insoluble polyvinyl alcohols, aliphatic and aromatic polyamides, polyimides, polyetherimides, polyesters, polycarbonates, polyolefins such as polyethylene, polypropylene, polyvinyl chloride, polyphenylene oxide, polybenzimidazoles and polybenzimidazolones, as well as from modifications, blends, mixtures, or copolymers derived from these polymers.

Other polymers can be mixed as additives with these polymers or polymer blends, for example polyethylene oxide, polyhydroxyether, polyethylene glycol, polyvinylpyrrolidone, polyvinyl alcohol, or polycaprolactone, or inorganic materials such as SiO₂. In individual cases, the membrane can also have been subjected to a surface modification, for example, in order to establish certain properties of the membrane surface such as in the form of certain functional groups. When using polyolefin polymers, it can be necessary to coat at least the inner surface of the membrane with a polymer permitting functionalization.

According to the fourth aspect of the present invention, the use of a photoswitchable compound is provided for isolating and/or purifying a target molecule.

Intriguingly, there is a lack of sufficient or enabling disclosure of a use of photoswitchable compounds for isolating and/or purifying a target molecule, as disclosed and described herein. Thus, the inventor and applicant should be granted acknowledgment of the first enabled, implementable and scalable solution to the problem of providing the use of photoswitchable compounds for isolating and/or purifying a target molecule from an aqueous solution comprising a mixture of different components.

In a preferred embodiment of the fourth aspect of the invention, the compound is not 4′-carboxyphenylazophenylalanine, more preferably the compound is not 3′-carboxyphenylazophenylalanine or 4′-carboxyphenylazophenylalanine, even more preferably the compound is not 3′-carboxyphenylazophenylalanine or a derivative thereof, and not 4′-carboxyphenylazophenylalanine or a derivative thereof.

In another preferred embodiment of the fourth aspect of the invention, the photoswitchable compound is a photoswitchable azobiaryl compound comprising at least two substituted biphenyl moieties.

In another preferred embodiment of the fourth aspect of the invention, the photoswitchable compound is a photoswitchable azobiaryl compound according to the first aspect of the invention.

According to the fifth aspect of the present invention, the use of a photoswitchable affinity ligand is provided comprising an affinity ligand in stable association with a photoswitchable compound for isolating and/or purifying a target molecule.

In a preferred embodiment of the fifth aspect of the invention, the photoswitchable compound is not 4′-carboxyphenylazophenylalanine, more preferably the compound is not 3′-carboxyphenylazophenylalanine or 4′-carboxyphenylazophenylalanine, even more preferably the compound is not 3′-carboxyphenylazophenylalanine or a derivative thereof, and not 4′-carboxyphenylazophenylalanine or a derivative thereof.

In another preferred embodiment of the fifth aspect of the invention, the photoswitchable affinity ligand comprises an affinity ligand in stable association with a photoswitchable azobiaryl compound comprising at least two substituted biphenyl moieties.

In a preferred embodiment of the fifth aspect of the invention, the photoswitchable affinity ligand is a photoswitchable affinity ligand according to the first aspect of the invention.

According to the sixth aspect of the present invention, the use of a photoswitchable affinity matrix comprising a solid support, and a photoswitchable compound in stable association with an affinity ligand, wherein the photoswitchable compound and the affinity ligand are in stable association with the solid support, or a photoswitchable affinity ligand in stable association with the solid support is provided for isolating and/or purifying a target molecule.

Again, such uses are not disclosed in the prior art and have not been successfully or enablingly identified and disclosed before the filing date of the present application.

In a preferred embodiment of the fifth or sixth aspect of the invention, the photoswitchable compound is not 4′-carboxyphenylazophenylalanine, more preferably the compound is not 3′-carboxyphenylazophenylalanine or 4′-carboxyphenylazophenylalanine, even more preferably the compound is not 3′-carboxyphenylazophenylalanine or a derivative thereof, and not 4′-carboxyphenylazophenylalanine or a derivative thereof.

In a preferred embodiment of the fifth or sixth aspect of the invention, the photoswitchable affinity ligand is a photoswitchable affinity ligand according to the second aspect of the invention. In one preferred embodiment of the sixth aspect of the present invention, the photoswitchable affinity matrix is the photoswitchable affinity matrix according to the third aspect of the present invention.

In a preferred embodiment of the fourth, fifth or sixth aspect of the invention, the target molecule is an immunoglobulin, more preferably the target molecule is an IgG type immunoglobulin, even more preferably an IgG-fragment or modality thereof (e.g. Fabs, diabodies (dAb), a single chain Fragment variable (scFv), a bispecific scFv (Bis-scFv), a ScFv-Fab, a Fc modified full IgG, a Dual-affinity Re-targeting Antibody (DART)) or fusion protein comprising the previously enumerated molecules.

According to the seventh aspect of the present invention, a method for isolating and/or purifying a target molecule is provided, wherein the method comprises the steps of providing a composition comprising a target molecule, contacting the composition with an affinity matrix comprising a photoswitchable compound in stable association with an affinity ligand and a solid support to form an affinity matrix, wherein, preferably, the compound is not 3′-carboxyphenylazophenylalanine or 4′-carboxyphenylazophenylalanine for a time sufficient to allow specific binding of the target molecule to the affinity matrix, washing the affinity matrix with a washing solution to remove the components of the composition not specifically bound to the affinity matrix, irradiating the affinity matrix with (a) wavelength(s) of light of at least about 400 nm in order to cause a loss of specific binding or affinity of the affinity matrix to the target molecule, and eluting the target molecule from the affinity matrix with an eluent.

In a preferred embodiment of the seventh aspect of the present invention, irradiating in step d) is conducted at (a) wavelength(s) in the range of about 400 nm to about 750 nm, preferably of about 400 nm to about 700 nm.

According to the eighth aspect of the present invention, a method of isolating and/or purifying a target molecule is provided, wherein the method comprises the steps of providing a composition comprising a target molecule, contacting the composition with an affinity matrix comprising a photoswitchable azobiaryl compound comprising at least two substituted biphenyl moieties, preferably the photoswitchable azobiaryl compound according to the first aspect of the invention in stable association with an affinity ligand, wherein the photoswitchable azobiaryl compound and the affinity ligand are in stable association with a solid support to form an affinity matrix; or a photoswitchable affinity ligand according to the second aspect of the invention in stable association with a solid support to form an affinity matrix; or a photoswitchable affinity matrix according to the third aspect of the present invention, for a time sufficient to allow specific binding of the target molecule to the affinity matrix, washing the affinity matrix with a washing solution to remove the components of the composition not specifically bound to the affinity matrix, irradiating the affinity matrix with (a) particular wavelength(s) of light in order to cause a loss of specific binding or affinity of the affinity matrix to the target molecule, and eluting the target molecule from the affinity matrix with an eluent.

In a preferred embodiment of the eighth aspect of the present invention, irradiating in step d) is conducted at (a) wavelength(s) of at least about 400 nm such as in the range of about 400 nm to about 750 nm, preferably of about 400 nm to about 700 nm.

According to a further aspect of the present invention, a method of isolating and/or purifying a target molecule is provided, wherein the method comprises the steps of providing a composition comprising a target molecule, contacting the composition with an affinity matrix comprising a photoswitchable compound in stable association with an affinity ligand and a solid support to form an affinity matrix, wherein, preferably, the compound is not 3′-carboxyphenylazophenylalanine or 4′-carboxyphenylazophenylalanine, for a time sufficient to allow specific binding of the target molecule to the affinity matrix, washing the affinity matrix with a washing solution to remove the components of the composition not specifically bound to the affinity matrix, irradiating the affinity matrix with (a) particular wavelength(s) of light in order to cause a loss of specific binding or affinity of the affinity matrix to the target molecule, and eluting the target molecule from the affinity matrix with an eluent.

All embodiments and aspects as described and/or claimed herein are deemed to be combinable within the present invention in any combination, unless the skilled person considers such a combination to not make any technical sense or to be excluded by contradiction.

### EXAMPLES

### Example 1: Synthesis Of 4′-(2-(4-(2-iodoacetamido)-3′,5′-dimethoxybiphenyl-3-ylcarboxamido)-acetic acid)-4′-(2-(4-(2-iodoacetamido)-3′,5′-dimethoxybiphenyl-3-ylcarboxamido)acetic acid)diazene (PS1)

When not otherwise stated, all chemicals and reagents were available from common suppliers (Carbolution, Sigma Aldrich, Alfa Aesar, Thermo Scientific, VWR). NMR spectra were recorded with Bruker Avance 500 spectrometer and Avance 500 spectrometer with QNP-Cryo. Coupling constants are reported in Hz. The compounds were characterized by the ¹H and ¹³C resonances from a set of 1D and 2D NMR experiments. ESI-MS analysis was performed with a Thermo Scientific LCQ-Fleet mass spectrometer coupled to a Thermo Scientific Dionex Ultimate 3000 HPLC system.

### Step 1: Synthesis of 2-(Tert-Butoxycarbonylamino)-5-Iodobenzoic Acid (1)

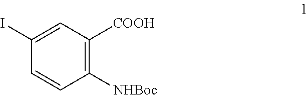 [SMILES: CC(C)(C)OC(=O)Nc1ccc(I)cc1C(=O)O]

26.3 g (100 mmol, 1 eq) 5-Iodoanthranilic acid were dissolved in 100 ml 1 M NaOH_{aq} and the pH was adjusted to 7-8 with additional 1 M NaOH_{aq} compared to universal indicator paper. 27.8 ml (130 mmol, 1.3 eq) Boc₂O were added and the mixture was stirred vigorously at RT (room temperature) for 8 h. The pH was adjusted to 7-8 a second time in the same manner as before. Additional 27.8 ml (130 mmol, 1.3 eq) Boc₂O were added and the mixture was stirred vigorously at RT for 16 h. The mixture was diluted with 300 ml H₂O, cooled in an ice bath and the pH was adjusted to 3-4 with 1 M HCl_{aq} to 3-4. The suspension was stirred for 30 min at 0° C. before the product was collected by vacuum filtration and was washed with 3 portions of H₂O. After air drying to constant weight 34.9 g (96 mmol, 96%) of a slightly beige solid were obtained.

¹H NMR (500 MHz, CDCl₃): δ [ppm]=9.94 (s, 1H, NH), 8.39 (d, Jₘₑₜₐ=2.2 Hz, 1H, C⁶Hₐᵣₒₘ), 8.28 (d, Jₒᵣₜₕₒ=9.1 Hz, 1H, C³Hₐᵣₒₘ), 7.81 (dd, Jₒᵣₜₕₒ=12.9 Hz, Jₘₑₜₐ=2.2 Hz, 1H, C4Hₐᵣₒₘ), 1.54 (s, 9H, Boc). ¹³C NMR (126 MHz, CDCl₃): δ [ppm]=171.5 (COOH), 152.6 (NHCOOC(CH₃)₃), 144.1 (C⁴ₐᵣₒₘ), 142.8 (C²ₐᵣₒₘ), 140.3 (C⁶ₐᵣₒₘ), 121.1 (C³ₐᵣₒₘ), 115.1 (C¹ₐᵣₒₘ), 83.4 (C⁵ₐᵣₒₘ), 81.5 (COOC(CH₃)₃), 28.4 (NHCOOC(CH₃)₃).

ESI-MS m/z (exact mass): calculated: 245.94 [M−Boc−OH]⁺, 263.95 [M−Boc+H]⁺, 363.00 [M+H]⁺, 1127.95 [3M+K]⁺; found: 245.96, 263.77, 362.00, 1127.96

### Step 2: Synthesis of Methyl 2-(2-(tert-butoxycarbonylamino)-5-iodobenzamido)acetate (2)

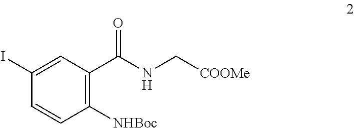 [SMILES: COC(=O)CNC(=O)c1cc(I)ccc1NC(=O)OC(C)(C)C]

To 2.542 g (7 mmol, 1 eq)N-Boc-5-iodoanthranilic acid (1) and 0.967 g (7.7 mmol, 1.1 eq) HCl·H-Gly-OMe 4.17 ml (24.5 mmol, 3.5 eq) DIPEA and 7 ml DMF were added and the mixture was stirred for 3 min before 4.007 g (7.7 mmol, 1.1 eq) PyBOP were added. After complete dissolution of the solids the reaction was stirred for 1 h at RT. 35 ml sat. NaHCO_{3 aq} were added and the mixture was stirred for 5 min. The mixture was extracted with 70 ml DCM and the organic phase was washed with 1×35 ml sat. NaHCO_{3 aq}, 2×35 ml 0.5 M HCl_{aq}, 1×35 ml brine, dried over MgSO₄, filtered and evaporated under vacuum to obtain 5.67 g crude product as clear yellow oil. The product was purified by column chromatography (SiO₂, ethylacetate/cyclohexane, step gradient 5%, 10%, 15% and 20%). The product containing fractions were combined and evaporated under reduced pressure to obtain 2.559 g (5.90 mmol, 84%).

¹H NMR (500 MHz, CDCl₃): δ [ppm]=9.93 (s, 1H, NHBoc), 8.17 (d, Jₒᵣₜₕₒ=9.0 Hz, 1H, C³Hₐᵣₒₘ), 7.78 (d, Jₘₑₜₐ=2.1 Hz, 1H, C⁶Hₐᵣₒₘ), 7.70 (dd, Jₒᵣₜₕₒ=8.9 Hz, Jₘₑₜₐ=2.1 Hz, 1H, C⁴Hₐᵣₒₘ), 6.67 (t, 3J=5.1 Hz, 1H, CONHCH₂), 4.20 (d, 3J=5.1 Hz, 2 H, CONHCH₂), 3.82 (s, 3H, COOCH₃), 1.50 (s, 9H, Boc).

¹³C NMR (126 MHz, CDCl₃): δ [ppm]=170.2 (COOMe), 167.6 (NHCOCH₂), 152.8 (NHCOOC(CH₃)₃) 141.6 (C⁴ₐᵣₒₘ), 140.3 (C²ₐᵣₒₘ), 135.4 (C⁶ₐᵣₒₘ), 121.9 (C³ₐᵣₒₘ), 121.0 (C¹ₐᵣₒₘ), 83.6 (C⁵ₐᵣₒₘ), 80.9 (COOC(CH₃)₃), 52.9 (COOCH₃), 41.7 (CH₂), 28.4 (NHCOOC(CH₃)₃).

ESI-MS m/z (exact mass): calculated: 245.94 [M-Boc-Gly]⁺, 334.99 [M−Boc+H]⁺, 457.02 [M+Na]⁺, 891.06 [2M+Na]⁺; found: 246.03, 334.84, 456.76, 890.99.

### Step 3: Synthesis of Methyl 2-(2-(Tert-Butoxycarbonylamino)-5-(4,4,5,5-Tetramethyl-1,3,2-dioxaborolan-2-yl)benzamido)acetate (3)

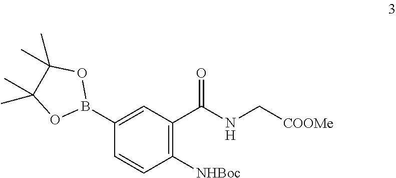 [SMILES: COC(=O)CNC(=O)c1cc(B2OC(C)(C)C(C)(C)O2)ccc1NC(=O)OC(C)(C)C]

The following reaction steps were performed under N₂-atmosphere. 11.8 ml degassed DMF (bubbled with N₂ for 30 min) were added to 2.554 g (5.88 mmol, 1 eq) 2, 2.240 g (8.82 mmol, 1.5 eq) bis(pinacolato)diboron and 1.731 g (17.64 mmol, 3 eq) potassium acetate (dried in high vacuum). The mixture was stirred and bubbled for 10 min with N₂ before 129 mg (0.18 mmol, 0.03 eq) Pd(dppf)Cl₂ were added. The mixture was stirred and bubbled for additional 10 min with N₂ before the reaction was stirred for 10 h at 90° C. in an oil bath with exclusion of light. After cooling to RT 25 ml of Et₂O was added and the mixture was filtered through a thin layer of celite which was washed with 13 ml Et₂O and 38 ml cyclohexane. The filtrate was washed 2× with 75 ml H₂O, 1× with 75 ml brine, dried over MgSO₄ and vacuum filtered through a pad of SiO₂. The pad was washed with 75 ml Et₂O/cyclohexane 1:1 and evaporated under reduced pressure to ≤10 ml. The solution was stored at 8° C. over night to obtain colorless white crystals which were carefully broken up with a spatula and collected by vacuum filtration. After washing the crystals with two small amounts of cold cyclohexane and air drying to constant weight 2.170 g (5.00 mmol, 85%) were obtained.

¹H NMR (500 MHz, CDCl₃): δ [ppm]=10.36 (s, 1H, NHBoc), 8.40 (d, Jₒᵣₜₕₒ=8.4 Hz, 1H, C³Hₐᵣₒₘ), 7.94 (d, Jₘₑₜₐ=1.4 Hz, 1H, C⁶Hₐᵣₒₘ), 7.86 (dd, Jₒᵣₜₕₒ=8.4 Hz, Jₘₑₜₐ=1.5 Hz, 1H, C⁴Hₐᵣₒₘ), 6.81 (t, 3J=5.3 Hz, 1H, CONHCH₂), 4.22 (d, 3J=5.2 Hz, 2 H, CONHCH₂), 3.81 (s, 3H, COOCH₃), 1.52 (s, 9H, Boc), 1.34 (s, 12H, Bpin).

¹³C NMR (126 MHz, CDCl₃): 0 [ppm]=170.3 (COOMe), 169.2 (NHCOCH₂), 152.9 (NHCOOC(CH₃)₃), 143.4 (C²ₐᵣₒₘ), 139.6 (C⁴ₐᵣₒₘ), 133.7 (Carom), 118.7 (C³ₐᵣₒₘ), 117.7 (C¹ₐᵣₒₘ), 84.13 (BO₂C₂ (CH₃)₄), 80.6 (COOC(CH₃)₃), 52.7 (COOCH₃), 41.7 (CH₂), 28.5 (NHCOOC(CH₃)₃), 25.0 (BO₂C² (CH₃)₄). (Signal of C⁵ₐᵣₒₘ which is directly attached to the boronic acid is not visible)

ESI-MS m/z (exact mass): calculated: 246.13 [M−Boc−Gly]⁺, 335.15 [M−Boc+H]⁺, 435.23 [M+H]+, 457.21 [M+Na]⁺, 891.43 [2M+Na]⁺; found: 246.19, 334.94, 434.72, 456.95, 891.42.

### Step 4: Synthesis of 4-bromo-2,6-dimethoxyaniline (4)

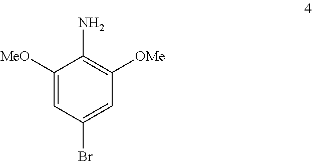 [SMILES: COc1cc(Br)cc(OC)c1N]

2.0 g (13.06 mmol, 1 eq) 2,6-dimethoxyaniline were dissolved in 26 ml acetic acid and 3.52 ml (30.04 mmol, 2.3 eq) 47% HBr_{aq} were added with stirring. 3.02 g (16.98 mmol, 1.3 eq)N-bromosuccinimide were added in one portion and the reaction was stirred for 1 h at RT. 125 ml DCM were added and the mixture was extracted 3× with 125 ml 1 M HCl_{aq}. The combined aqueous extracts were cooled to 0° C., made strongly alkaline with 50% NaOH_{aq} while stirring and extracted 3× with 50 ml DCM. The combined organic extracts were dried over MgSO₄ and filtered through a tightly packed pad of SiO₂ in a sintered glass funnel which was washed with additional 100 ml DCM. The filtrate was evaporated under reduced pressure to obtain 1.98 g (8.50 mmol, 65%) of a slightly brown-reddish solid, which was first an oil and crystallized spontaneously at RT.

¹H NMR (500 MHz, CDCl₃): δ [ppm]=6.65 (s, 2H, C^{3,5}Hₐᵣₒₘ), 3.83 (s, 6H, 2×OCH₃). (Signal of NH₂ is not visible in CDCl₃).

¹³C NMR (126 MHz, CDCl₃): δ [ppm]=147.8 (C^{2,6}ₐᵣₒₘ), 124.7 (C¹ₐᵣₒₘ), 108.8 (C⁴ₐᵣₒₘ), 107.7 (C^{3,5}ₐᵣₒₘ), 56.1 (2×OCH₃).

ESI-MS m/z (exact mass): calculated: 232.00 [M(⁷⁹Br)+H]⁺, 234.00 [M(⁸¹Br)+H]⁺, found: 232.06, 233.96.

### Step 5: Synthesis of Methyl 2-(4′-amino-4-(tert-butoxycarbonylamino)-3′,5′-dimethoxybiphenyl-3-ylcarboxamido)acetate (5)

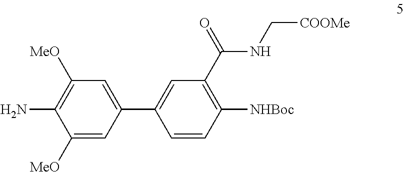 [SMILES: COC(=O)CNC(=O)c1cc(-c2cc(OC)c(N)c(OC)c2)ccc1NC(=O)OC(C)(C)C]

The following reaction steps were performed under N₂-atmosphere. 1.950 g (4.49 mmol, 1 eq) 3, 1.033 g (4.49 mmol, 1 eq) 4 and 1.870 g K₂CO₃ (13.47 mmol, 3 eq) were dissolved in 22.5 ml degassed toluene (bubbled with N₂ for 30 min) and 11.3 ml H₂O (bubbled with N₂ for 30 min). The mixture was stirred and bubbled with N₂ for 10 min before 197 mg (0.27 mmol, 0.06 eq) Pd(dppf)Cl₂ were added. The biphasic mixture was stirred and bubbled with N₂ for additional 10 min before it was stirred at 90° C. in an oil bath for 5 h. After the reaction was cooled to RT it was diluted with 200 ml ethyl acetate and stirred till most of the solids dissolved. The biphasic mixture was vacuum filtered through a thin pad of celite and washed with 25 ml ethyl acetate/toluene 9:1 and 125 ml H₂O. The filtrate is shaken and the phases are separated. The organic layer was washed once more with 125 ml H₂O, 1× with 125 ml brine, dried over MgSO₄ and filtered through a tightly packed pad of SiO₂ in a sintered glass funnel which was washed with additional 100 ml ethyl acetate/toluene 9:1. The filtrate was evaporated under reduced pressure to obtain a brown solid with an oily character (ca. 2 g). The solid was completely dissolved in refluxing isopropanol. The solution was evaporated under reduced pressure at 50° C. to app. 10 ml (everything stayed in solution), heated to reflux with stirring before 50 ml of hot hexane were added while stirring. Everything was stirred under reflux for 5 min before it was cooled to RT and kept at 4° C. over night. The solid was collected by vacuum filtration, washed with two small amounts of cold hexane and air dried to obtain 1.052 g (2.29 mmol, 51%) of a beige solid.

¹H NMR (500 MHz, CDCl₃): 0 [ppm]=9.96 (s, 1H, NHBoc), 8.39 (d, Jₒᵣₜₕₒ=8.7 Hz, 1H, C⁵Hₐᵣₒₘ), 7.65-7.56 (m, 2H, C^{2,6}Hₐᵣₒₘ), 6.83 (t, 3J=5.1 Hz, 1H, CONHCH₂), 6.68 (s, 2H, C^{2,6′}Hₐᵣₒₘ), 4.24 (d, 3J=5.0 Hz, 2 H, CONHCH₂), 3.92 (s, 6H, OCH₃), 3.81 (s, 3H, COOCH₃), 1.52 (s, 9H, Boc). (Signal of NH₂ is not visible in CDCl₃)

¹³C NMR (126 MHz, CDCl₃): δ [ppm]=170.4 (COOMe), 169.2 (NHCOCH₂), 153.1 (NHCOOC(CH₃)₃), 147.8 (C^{3′,5′}ₐᵣₒₘ), 138.9 (C⁴ₐᵣₒₘ), 135.6 (C¹ₐᵣₒₘ), 131.3 (C⁶ₐᵣₒₘ), 129.3 (C^{1′}ₐᵣₒₘ), 125.2 (C³ₐᵣₒₘ), 125.2 (C³ₐᵣₒₘ), 125.0 (C²ₐᵣₒₘ), 120.3 (C⁵ₐᵣₒₘ), 119.4 (C^{4′}ₐᵣₒₘ), 103.1 (C^{2′,6′}ₐᵣₒₘ), 80.5 (COOC(CH₃)₃), 56.2 (2×OCH₃) 52.7 (COOCH₃), 41.8 (CH₂), 28.5 (NHCOOC(CH₃)₃).

ESI-MS m/z (exact mass): calculated: 360.15 [M−Boc+H]⁺, 460.21 [M+H]⁺, 919.41 [2M+H]⁺; found: 359.91, 460.01, 919.32.

### Step 6a: Synthesis of Mn^{III}SalophenCl

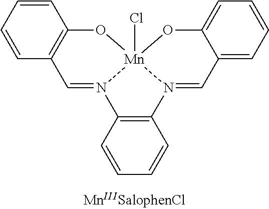

2.163 g (20 mmol, 1 eq) phenylendiamine and 4.175 ml (40 mmol, 2 eq) salicylaldehyde were dissolved in 40 ml EtOH and were refluxed with stirring for 2 h. The reaction containing an orange crystalline precipitate was diluted with 460 ml EtOH, 6.921 g (40 mmol, 2 eq) Mn(OAc) 2 were added and reaction was refluxed with stirring for 5 h under a slight stream of air introduced through the condenser. 1.696 g (40 mmol, 2 eq) LiCl were added and stirring under reflux was continued for additional 2 h. The mixture was evaporated under reduced pressure to an app. volume of 100 ml, 500 ml H₂O were added while stirring and the mixture was stirred vigorously for 30 min at RT. The precipitate was collected by vacuum filtration, washed three times with water and air dried to yield 6.050 g (14.95 mmol, 75%) of a pale brown solid. The product was only characterized by its catalytic activity according to Mirkhani et al., Biorg Med Chem (2004), 12, 4376-7.

### Step 6b: Synthesis of (E)-4′-(methyl 2-(4-(tert-butoxycarbonylamino)-3′,5′-dimethoxybiphenyl-3-ylcarboxamido)acetate)-4′-(methyl 2-(4-(tert-butoxycarbonylamino)-3′,5′-dimethoxybiphenyl-3-ylcarboxamido)acetate)diazene (6)

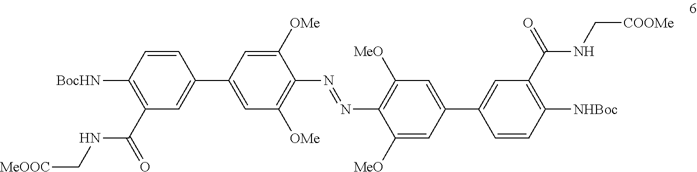 [SMILES: COC(=O)CNC(=O)c1cc(-c2cc(OC)c(/N=N/c3c(OC)cc(-c4ccc(NC(=O)OC(C)(C)C)c(C(=O)NCC(=O)OC)c4)cc3OC)c(OC)c2)ccc1NC(=O)OC(C)(C)C]

### Synthetic Procedure:

19.3 ml MeCN were added to 0.890 g (1.94 mmol, 1 eq) 5, 39.2 mg (97.00 μmol, 0.05 eq) Mn^{III}′SalophenCl and 6.6 mg (97.00 μmol, 0.05 eq) imidazole. The mixture was stirred 5 min before 0.829 g (3.88 mmol, 2 eq) NaIO₄ dissolved in 9.7 ml H₂O were added and the reaction was stirred for 90 min at RT. 80 ml ethyl acetate were added, stirring was continued for 5 min before the biphasic mixture was filtered through celite which was washed twice with 10 ml ethyl acetate each. The filtrate was washed 2× with 50 ml H₂O, 1× with 50 ml brine, dried over MgSO₄, filtered and evaporated under reduced pressure to yield the crude product as a dark solid. To a solution of the crude product in 5 ml DCM were added 50 ml MeOH while stirring. The mixture was kept at −20° C. overnight, the precipitate was collected by vacuum filtration, washed twice with a small amount of cold MeOH. After air drying 0.453 g (0.50 mmol, 51%) of a dark red solid were obtained. The mother liquor was evaporated to dryness under reduced pressure and the residue was subjected to column chromatography (SiO₂, ethylacetate/cyclohexane (2:1)+1% AcOH). Evaporation of the solvent of product containing fractions under reduced pressure yielded additional 0.275 g (0.30 mmol, 31%) of the product as a burgundy red solid and an overall yield of 0.728 g (0.80 mmol, 82%).

¹H NMR (500 MHz, CDCl₃): 0 [ppm]=10.48 (s, 2H, NHBoc), 8.49 (d, Jₒᵣₜₕₒ=8.8 Hz, 2 H, C⁵Hₐᵣₒₘ), 7.92 (d, Jₘₑₜₐ=2.2 Hz, 2 H, C²Hₐᵣₒₘ), 7.75 (t, 3J=5.4 Hz, 2 H, CONHCH₂), 7.62 (dd, Jₒᵣₜₕₒ=8.8 Hz, Jₘₑₜₐ=2.1 Hz, 2 H, C⁶Hₐᵣₒₘ) 6.64 (s, 4H, C^{2,6′}Hₐᵣₒₘ), 3.85 (d, 3J=5.2 Hz, 4 H, CONHCH₂), 3.70 (s, 12H, OCH₃), 3.67 (s, 6H, COOCH₃), 1.52 (s, 18H, Boc).

¹³C NMR (126 MHz, CDCl₃): 0 [ppm]=170.2 (2×COOMe), 169.2 (2×NHCOCH₂), 153.1 (2×C^{3′,5′}ₐᵣₒₘ), 152.8 (2×NHCOOC(CH₃)₃), 142.3 (2×C⁴ₐᵣₒₘ), 140.9 (2×C¹ₐᵣₒₘ), 133.4 (2×C^{1′}ₐᵣₒₘ), 132.2 (2×C³ₐᵣₒₘ), 131.4 (2×C⁶ₐᵣₒₘ), 126.2 (2×C²ₐᵣₒₘ), 119.8 (2×C⁵ₐᵣₒₘ), 118.7 (2×C⁴ₐᵣₒₘ), 103.2 (2×C^{2,6′}ₐᵣₒₘ), 80.6 (2×COOC(CH₃)₃), 56.0 (4×OCH₃) 52.4 (2×COOCH₃), 41.4 (2×CH₂), 28.5 (2×NHCOOC(CH₃)₃).

ESI-MS m/z (exact mass): calculated: 915.37 [M+H]⁺, 1829.74 [2M+H]⁺; found: 915.37, 1829.05.

### Step 7: Synthesis of (E)-4′-(2-(4-amino-3′,5′-dimethoxybiphenyl-3-ylcarboxamido)acetic acid)-4′-(2-(4-amino-3′,5′-dimethoxybiphenyl-3-ylcarboxamido)acetic acid)diazene (7)

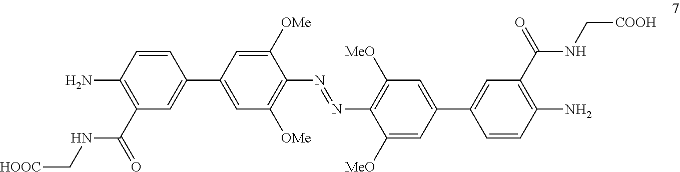 [SMILES: COc1cc(-c2ccc(N)c(C(=O)NCC(=O)O)c2)cc(OC)c1/N=N/c1c(OC)cc(-c2ccc(N)c(C(=O)NCC(=O)O)c2)cc1OC]

321 μl acetyl chloride (4.5 mmol, 30 eq) were added to a solution of 182 μl (4.5 mmol, 30 eq) dry MeOH in 1.15 ml ethyl acetate at 0° C. The solution was left at 0° C. for 5 min and was then added to 137.2 mg (0.15 mmol, 1 eq) 6 dissolved in 1.5 ml dry DCM at RT. The reaction was stirred at RT for 2.5 h. The precipitated dianilinium chloride was collected by centrifugation, the supernatant was decanted and the solid was washed twice with DCM by resuspension, centrifugation and decantation. After air drying and drying under reduced pressure the dianilinium chloride was obtained in quantitative yield as a blue-purple solid and was directly used in the next step.

Therefore, the solid was dissolved/suspended in 1.5 ml THF/MeOH (1:1) containing 150 μl (0.3 mmol, 2 eq) 2 M NaOH_{aq} and the mixture was stirred for 5 min, while stirring 1.35 ml (2.7 mmol, 18 eq) 2 M NaOH_{aq} were added, and the reaction was stirred for 1 h at RT. Then 1.5 ml H₂O were added, the reaction was stirred for additional 1.5 h, then 6 ml H₂O were added, and the reaction was stirred for additional 7 h. Afterwards everything was dissolved by the addition of 7.5 ml H₂O, the pH was adjusted to ~4 with 1 M HCl_{aq} compared to universal indicator paper and the precipitate was collected by centrifugation. The supernatant was decanted and the solid was washed 3× with 5 ml H₂O each by reuspension, centrifugation and decantation. After drying in high vacuum, the product was obtained as a dark blue extremely electrostatic solid in quantitative yield (0.15 mmol, 103.0 mg).

The product was directly used in the next step without further purification or characterization.

### Step 8: Synthesis of (E)-4′-(benzhydryl 2-(4-amino-3′,5′-dimethoxybiphenyl-3-ylcarboxamido)acetate)-4′-(benzhydryl 2-(4-amino-3′,5′-dimethoxybiphenyl-3-ylcarboxamido)acetate)diazene (8)

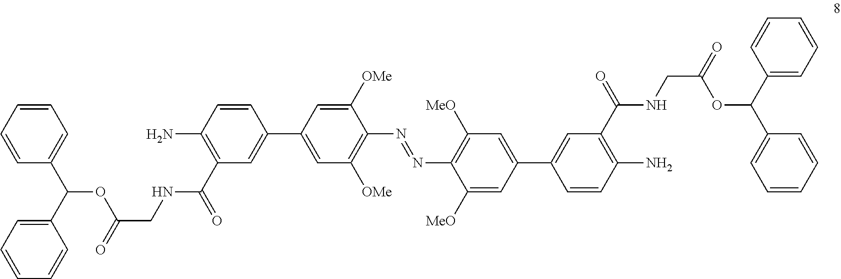 [SMILES: COc1cc(-c2ccc(N)c(C(=O)NCC(=O)OC(c3ccccc3)c3ccccc3)c2)cc(OC)c1/N=N/c1c(OC)cc(-c2ccc(N)c(C(=O)NCC(=O)OC(c3ccccc3)c3ccccc3)c2)cc1OC]

103 mg (0.15 mmol, 1 eq) 7 were dissolved in 750 μl DMF. 750 μl THF were added to this solution with stirring and subsequently 450 μl (0.9 mmol, 6 eq) 2 M diazodiphenylmethane (Javed and Brewer, Org. Synth. (2008), 85, 189, DOI: 10.15227/orgsyn.085.0189) diazodiphenylmethane in THF. The reaction was stirred for 15 h at RT before 225 μl (0.45 mmol, 3 eq) 2 M diazodiphenylmethane in THF were added and the reaction was stirred for additional 7 h. Afterwards 150 μl (0.3 mmol, 2 eq) 2 M diazodiphenylmethane in THF were added and the reaction was stirred for additional 2 h before 25 ml cold Et₂O were added to precipitate the product. Precipitation was completed at −20° C. for 1 h, the dark blue solid was collected by centrifugation, the supernatant was decanted and the solid was washed 2×with cold Et₂₀ by resuspension, centrifugation and decantation. The solid was subjected to column chromatography (SiO₂, DCM/MeOH, step gradient of 0, 2, 4, . . . , 20% MeOH). The product containing fractions were evaporated under reduced pressure to leave 122 mg of a blue-reddish solid which consists of the desired product and a significant amount of the product which had formed an imine with benzophenone (traces of diimine were also present). The solid was dissolved in 750 μl DMF, 750 μl THF were added to the solution with stirring and subsequently 1.5 ml 95% AcOH_{aq} were added. The mixture was stirred for 15 h at RT before 30 ml cold Et₂O were added to precipitate the product. Precipitation was completed at −20° C. for 1 h, the dark blue solid was collected by centrifugation, the supernatant was decanted and the solid was washed 2× with cold Et₂O by resuspension, centrifugation and decantation. After residual solvent was removed in vacuum 107 mg (104.99 μmol, 70%) of the product as dark blue solid was obtained without the imine byproduct.

¹H NMR (500 MHz, CDCl₃): 0 [ppm]=7.73 (d, Jₘₑₜₐ=2.1 Hz, 2 H, C²Hₐᵣₒₘ), 7.39 (dd, Jₒᵣₜₕₒ=8.4 Hz, Jₘₑₜₐ=2.1 Hz, 2 H, C⁶Hₐᵣₒₘ), 7.36-7.29 (m, 2H, CONHCH₂), 7.28-7.19 (m, 20H, DPMₐᵣₒₘ), 6.83 (s, 2H, PhCHPh), 6.69 (d, Jₒᵣₜₕₒ=8.5 Hz, 2 H, C⁵Hₐᵣₒₘ), 6.55 (s, 4H, C^{2′,6}′Hₐᵣₒₘ), 5.82-5.52 (bs, 4 H, NH₂), 3.96 (d, 3J=5.2 Hz, 4 H, CONHCH₂), 3.62 (s, 12H, OCH₃).

¹³C NMR (126 MHz, CDCl₃): δ [ppm]=169.6 (2×COODPM), 169.4 (2×NHCOCH₂), 152.9 (2×C^{3′,5′}ₐᵣₒₘ), 148.9 (2×C⁴ₐᵣₒₘ), 142.6 (2×C¹ₐᵣₒₘ), 139.8 (2×DPM), 132.1 (2×C^{1′}ₐᵣₒₘ), 131.6 (2×C⁶ₐᵣₒₘ), 129.1 (2×C³ₐᵣₒₘ), 128.7 (2×DPMₒᵣₜₕₒ), 128.2 (2×DPMₘₑₜₐ), 127.2 (2×DPMₚₐᵣₐ), 126.8 (2×C²ₐᵣₒₘ), 117.4 (2×C⁵ₐᵣₒₘ), 115.3 (2×C^{4′}ₐᵣₒₘ), 102.7 (2×C^{2,6′}ₐᵣₒₘ), 78.0 (2×PhCHPh), 55.9 (4×OCH₃), 41.7 (2×CH₂).

ESI-MS m/z (exact mass): calculated: 1019.39 [M+H]⁺; found: 1019.68.

### Step 9: Synthesis of (E)-4′-(2-(4-(2-iodoacetamido)-3′, 5′-dimethoxybiphenyl-3-ylcarboxamido)acetic acid)-4′-(2-(4-(2-iodoacetamido)-3′,5′-dimethoxybiphenyl-3-ylcarboxamido)acetic acid)diazene (PS1)

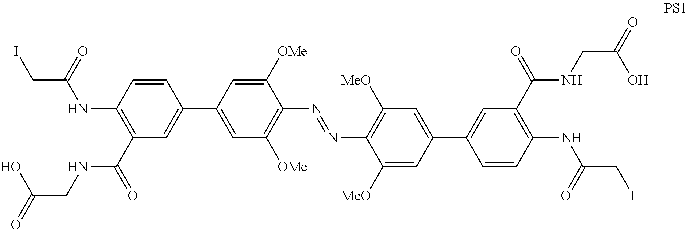 [SMILES: COc1cc(-c2ccc(NC(=O)CI)c(C(=O)NCC(=O)O)c2)cc(OC)c1/N=N/c1c(OC)cc(-c2ccc(NC(=O)CI)c(C(=O)NCC(=O)O)c2)cc1OC]

102 mg (100.10 μmol, 1 eq) 8 were dissolved in 2 ml dry DCM, 34.7 mg (0.25 mmol, 2.5 eq) K₂CO₃ were added, the mixture was stirred and cooled to 0° C. 18.4 μl (205.21 μmol, 2.05 eq) iodoacetyl chloride were added and the reaction was stirred for 1 h at 0° C. Then additional 1.34 μl (15.02 μmol, 0.15 eq) iodoacetyl chloride were added and the mixture was stirred for additional 1 h at 0° C. before once more 1.79 μl (20.03 μmol, 0.2 eq) iodoacetyl chloride were added. After stirring for additional 1 h at 0° C. the reaction was quenched by addition of 1 ml H₂O, the mixture was diluted with 18 ml DCM, washed 3×with 10 ml H₂O by cautious swirling and shaking to prevent emulsion formation, the organic phase was dried over MgSO₄ and filtered through a thin layer of celite which was washed with DCM till the filtrate was colorless. Evaporation of the solvent under reduced pressure led to a dark red to purple solid which was dissolved in 1.5 ml DCM with 0.1 ml anisole. The solution was cooled to 0° C. before 0.4 ml TFA were added while mixing. The reaction was kept at 0° C. for 2 h before 20 ml of cold Et₂O ware added to precipitate the product. Precipitation was completed for 1 h at −20° C., the dark purple solid was collected by centrifugation, the supernatant was decanted and the solid was washed 2× with cold Et₂O by resuspension, centrifugation and decantation. After air drying and drying in vacuum 102 mg (99.73 μmol, 99.6%) of a dark purple solid was obtained.

¹H NMR (500 MHz, DMSO-d6): δ [ppm]=11.95 (s, 1H, NHAcl), 11.47 (s, 1H, NHAcl), 9.37 (t, 3J=5.8 Hz 1 H, CONHCH₂), 9.32 (t, 3J=5.8 Hz 1 H, CONHCH₂), 8.76 (d, Jₒᵣₜₕₒ=8.7 Hz, 1H, C⁵Hₐᵣₒₘ), 8.48 (d, Jₒᵣₜₕₒ=8.7 Hz, 1H, C⁵Hₐᵣₒₘ), 8.18-8.11 (m, 2H, C²Hₐᵣₒₘ), 8.07-7.99 (m, 2H, C⁶Hₐᵣₒₘ), 7.18-7.13 (m, 4H, C^{2,6′}Hₐᵣₒₘ), 4.06-4.02 (m, 4H, CH₂COOH), 4.01-3.97 (m, 4H, CH₂₁), 3.87 (s, 12H, OCH₃).

¹³C NMR (126 MHz, DMSO-d6): δ [ppm]=171.6 (COOH), 171.2 (2×NHCO), 168.2 (2×NHCO), 158.5 (2×C¹ₐᵣₒₘ), 158.2 (2×C^{1′}ₐᵣₒₘ), 152.1 (2×C^{3′,5′}ₐᵣₒₘ), 138.4 (2×C⁴ₐᵣₒₘ), 133.9 (2×C³ₐᵣₒₘ), 132.7 (2×C^{4′}ₐᵣₒₘ), 130.7 (2×C⁶ₐᵣₒₘ), 126.6 (2×C²ₐᵣₒₘ), 120.4 (2×C⁵ₐᵣₒₘ), 103.9 (2×C^{2,6′}ₐᵣₒₘ), 62.1 (2×CH₂l), 56.6 (4×OCH₃), 41.2 (2×CH₂COOH).

ESI-MS m/z (exact mass): calculated: 1023.06 [M+H]⁺, found: 1022.81

### Example 2: Coupling to Cysteine Side Chains and Light-Induced Isomerization of 4′-(2-(4-(2-iodoacetamido)-3′,5′-dimethoxybiphenyl-3-ylcarboxamido)acetic acid)-4′-(2-(4-(2-iodoacetamido)-3′,5′-dimethoxybiphenyl-3-ylcarboxamido)acetic acid)diazene (PS1)

The UV-VIS absorption spectrum of azobenzene reveals two characteristic absorption bands corresponding to π-π* and π-π* electronic transitions, which differ in amplitude and precise location of the absorption maximum (A) for the trans- and cis-configuration. The electronic transition π-π* is usually in the near UV region around 340 nm (Sension et al. 1993) whereas the electronic transition π-π* is usually located in the visible (VIS) region around 420 nm and is due to the presence of unshared electron pairs of the nitrogen atoms (Nägele et al. 1997).

A key feature of the claimed photoswitchable azobenzene compound designed to generate a photoswitchable affinity matrix is the wavelength of light required to cause the photoisomerization and thus modulation of the affinity. Introducing electron-donating or push/pull substituents at the para positions delocalizes the azobenzene chromophore and leads to long wavelength absorption but usually also lowers the thermal barrier to interconversion of the isomers (Dong et al. 2015). Fast thermal relaxation means it is difficult to produce a large steady state fraction of the cis-isomer. Thus, specifically preserve binding activity of a photoswichtable affinity ligand with the cis-isomer would require an impractically bright light source. Introducing substituents at all four ortho positions leads to azo compounds with several unusual properties that are useful for the generation of a photoswitchable affinity matrix. Tetra-ortho substituted azo compounds show unusually slow thermal relaxation rates and enhanced separation of n-π* transitions of cis- and trans-isomers compared to analogues without ortho substituents. Ortho methoxy groups greatly stabilize the azonium form of the compounds, in which the azo group is protonated. Azonium ions absorb strongly in the red region of the spectrum and can reach into the near-IR. These azonium ions can exhibit robust cis-trans isomerization in aqueous solutions at neutral pH. By varying the nature of ortho substituents, together with the number and nature of meta and para substituents, long wavelength switching, stability to photobleaching, stability to hydrolysis, and stability to reduction by thiols can all be crafted into a photoswitch (Dong et al. 2015).

### HPLC and Spectral Analysis:

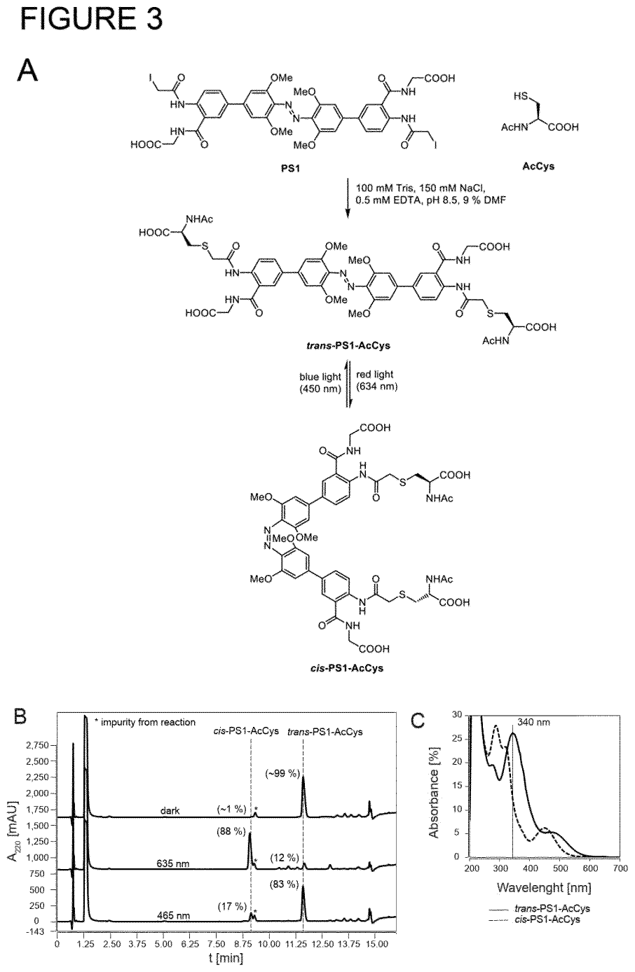

To examine whether the synthesized PS1 can respond to photoswitching induced by visible light, when attached to cysteine sidechains, the compound was coupled with N-acetyl cysteines (FIG. 3A) and subjected to alternating irradiation cycles with subsequent analysis.

In a typical experiment performed at 25° C. 1.0 mg of PS1 were dissolved in 97 μl DMF with exclusion of light to obtain a 10 mM solution. 0.5 μl of this solution was analyzed by HPLC using method A (data not shown). 10 μl of this solution were combined with 100 μl of a 5 mM solution N-acetyl cysteine in an aqueous buffer (100 mM Tris/Cl, 150 mM NaCl, 0.5 EDTA, pH 8.5). The mixture was shaken for 30 min in the dark. 5.5 μl each of this solution was analyzed by HPLC using method A (data not shown) and method B. The residual solution was illuminated with red light (LED-635 nm; NCSR219B-V1, Nichia, Tokushima, Japan) for 2 min and 5.5 μl were analyzed by HPLC using method B (FIG. 3B). Afterwards this solution was illuminated with blue light (LED-465 nm; NCSR219B-V1, Nichia, Tokushima, Japan) for 2 min and 5.5 μl were analyzed by HPLC using method B (FIG. 3B).

The chromatogram of the upper panel in FIG. 3B reveals mostly the trans-isomer when adapted in the dark; the chromatogram in the middle panel reveals mostly the cis-isomer, with the trans-isomer as minor species when illuminated with 635 nm; the chromatogram in the lower panel reveals mostly trans-isomer, with the cis-isomer as minor species when illuminated with 465 nm.

To determine t_{1/2}(cis→trans) at 25° C., the solution was again illuminated with red light for 2 min, kept in the dark and 5.5 μl each were analyzed by HPLC method B at 0, 20, 40, . . . , 120 min. t_{1/2} (cis→trans) was determined to be >20 h at 25° C. (data not shown). UV-Vis spectra, HPLC-chromatograms and isomer ratios were recorded using a Thermo Scientific Dionex Ultimate 3000 HPLC system with Diode Array Detector.

### Method A:

Column: Agilent Zorbax Eclipse® XDB-C⁸4.6×150 mm 5-Micron, eluent A: 10 mM NH₄OAc in H₂O, eluent B: 10 mM NH₄OAc in H₂O/MeCN(1:9), gradient: 5→5% B (1 min) 5→95% B (12.5 min) 95→95% B (3.5 min), flow: 2 ml/min.

### Method B:

Column: Agilent Zorbax Eclipse® XDB-C8 4.6×150 mm 5-Micron, eluent A: 10 mM NH₄OAc in H₂O, eluent B: 10 mM NH₄OAc in H₂O/MeCN(1:9), gradient: 5→5% B (1 min) 5→20% B (12.5 min) 20→95% B (0.1 min), 95→95% B (3.4 min), flow: 2 ml/min. Ratios of the cis- and trans-isomeres were determined by peak integration at 220 nm.

The change in intensity of the π-π* band at around 340 nm corresponds to photoswitching between the trans-(high absorbance at 340 nm) and cis-(low absorbance at 340 nm) configuration of PS1-AcCys in aqueous buffer (FIG. 3C). High absorption at 340 nm indicates the trans-configuration whereas low absorption at 340 nm indicates the cis-configuration.

FIG. 3B shows the corresponding HPLC chromatograms with absorbance at λ=220 nm (wavelength at which trans-PS1-AcCys and cis-PS-AcCys show the same molar extinction coefficient, allowing direct comparison of peak integrals). The chromatograms reveal that the cis- and trans-isomers of PS1-AcCys can be separated by HPLC (cis-PS1-AcCys t_{R}=9.1 min, trans-PS1-AcCys t_{R}=11.6 min). Irradiation with red light causes an increase in the proportion of cis-PS1-AcCys, here up to 88% (FIG. 3B), which can be reversed by irradiation with blue light, thus recovering the ground state via photochemical reisomerization.

Thus, if the photoswitchable affinity matrix of the present invention is applied for an affinity chromatography procedure, the cis-configuration may correspond to the high affinity state whereas the trans-configuration may correspond to the low affinity conformation. High states of occupation of the respective configurations were achievable by illumination with light of 635 nm and 465 nm, respectively.

### Example 3: Generation OF SpA VARIANT SpA #1

The Ig-binding protein of the present invention, as used in this example, comprises the B domain of protein A (SEQ ID NO. 4), defined as residues 215-268 corresponding to UniProtKB entry P38507 (SEQ ID NO. 1). Residues 215-216 in SEQ ID NO. 4 were replaced by Lys213-Ala214-Cys215-Gly216 (resulting in SEQ ID NO. 6) and the single substitution Ser250Cys was further introduced, resulting in SpA #1 (SEQ ID NO. 7). The sequence of the Ig-binding affinity ligand SpA #1 was extended at the N-terminus by Met212 and at the C-terminus by Ser269-Ala270-His271-His272-His273-His274-His275-His276.

To generate SpA variants suitable for the derivatization with PS1, the DNA sequence encoding SEQ ID NO. 4 was altered using a PCR assembly approach. First, two separate DNA fragments were generated, sharing overlapping sequences that carry the respective mutation. In a final PCR reaction, these two fragments were assembled to create a mutated DNA sequence, poised to serve as a PCR template for the introduction of further mutations or to be sub-cloned in the expression vector.

To generate SpA #1, two consecutive mutations were introduced by the aforementioned approach. Using the wild type DNA sequence as a PCR template, two pairs of forward and reverse primers were used to introduce the N-terminal protein sequence alteration. Fragments were synthesized in a 20 μl PCR reaction, including 1×Phusion HF buffer, 0.5 UM of forward and reverse primer, 5 ng template DNA, 200 UM of each dNTP and 0.02 U Phusion DNA polymerase per μl reaction mixture (New England Biolabs, Ipswich, Mass., USA). After initial DNA denaturation at 98° C. for 180 s, 30 cycles of PCR, comprising the steps denaturation (10 s, 98° C.), primer annealing (10 s, 61° C.) and polymerization (15 s, 72° C.), were performed using a thermocycler. The reaction was completed after final 180 s at 72° C. Fragments were assembled in a second 50 μl PCR reaction, using 1 μl of each fragment reaction mixture as template DNA and the flanking forward and reverse primers. Reaction conditions were the same as before. All primers were supplied by MWG Eurofins (Ebersberg, Germany). The final PCR product was purified using QIAquick PCR Purification Kit (Qiagen, Hilden, Germany).

The PCR fragment described above served as a template for the introduction of the second mutation (Ser250Cys) by a similar approach. Fragments were generated using forward and reverse primer pairs. The final PCR product was assembled using the flanking forward and reverse primers.

The mutated DNA fragment, encoding SpA #1, was sub-cloned in the modified expression vector backbone based on pD451sr (ATUM, Newark, CA, USA) using the flanking DNA restriction sites Xba I and Hind III. Target vector and DNA insert were digested with the respective enzymes in a 50 μl reaction mixture (5 μg DNA; 1×CutSmart buffer; 0.04 U/μl Xba I; 0.04 U/μl Hind III-HF) and were incubated 60 min at 37° C. Digested DNA fragments were purified using a 1% (w/v) agarose gel and extracted using the QIAquick Gel Extraction Kit (Qiagen, Hilden, Germany). Vector backbone and DNA insert were ligated in a 20 μl ligation reaction mixture (1×T4 DNA ligase reaction buffer; 100 ng purified and linearized vector; 3 molar equivalents of purified DNA insert; 0.05U/μl T4 DNA ligase), incubated 20 min at 20° C. Transformation (Inoue et al. 1990) of chemical competent E. coli NEB Turbo cells (New England Biolabs, Ipswich, Mass., USA) was performed by adding 5 μl of ligation reaction mixture to 50 μl cell suspension, incubation on ice for 15 min and followed by a heat shock 30 s at 42° C. and 30 s on ice. After isolation of plasmid DNA from single clone transformants, the correct sequence of the resulting expression plasmid pD451sr-SpA #1 was confirmed by Sanger sequencing.

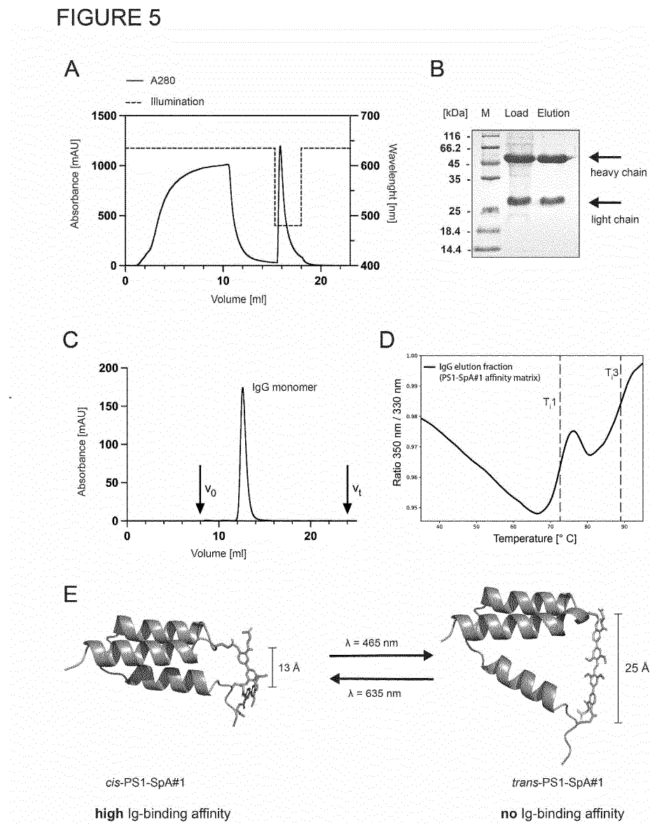

The residues substituted with Cys were intended to provide attachment points for PS1 and preserve antibody binding when the photoswitch adopts the cis-conformation (i.e., after illumination at 635 nm) but disturb binding in the trans-conformation (i.e., after illumination at 465 nm) (FIG. 5E). Position Cys215 is located at the N-terminus of the three-helix bundle, position Cys250 is located in the loop between helix 2 and 3. Isomerization of PS1 to the trans-state was supposed to change the overall protein conformation and thus the overall geometry necessary for the interaction with an antibody molecule.

### Example 4: Expression and Purification of Spa #1

The SpA variant SpA #1 was produced recombinantly as a soluble protein in the cytoplasm of E. coli, isolated with high yields by immobilized metal ion affinity chromatography (IMAC) via the C-terminal 6×His-tag, purified by size-exclusion chromatography (SEC) and analyzed by SDS-PAGE.

First, a single colony of E. coli NEB T7 Express transformed with the expression plasmid pD451sr-SpA #1, encoding SpA #1 (SEQ ID NO. 7), was used to inoculate 3 ml LB medium supplemented with 50 μg/ml kanamycin. After incubation overnight at 37° C. and sufficient agitation, 2 ml culture suspension were diluted in 200 ml LB medium in a 1 L shake flask, again supplemented with 50 μg/ml kanamycin. The production culture was cultivated at 37° C. and 250 rpm at 25 mm stroke of orbital motion until an optical density at 600 nm (OD 600) of 0.5 was reached. At this point, the T7 promoter regulated expression of SpA #1 was induced using 0.5 mM isopropyl B-d-1-thiogalactopyranoside (IPTG) and was continued for additional 4 h with unchanged growth conditions. At the end of fermentation, E. coli cells were harvested by centrifugation (12,000×rcf, 10 min, 4° C.). The sedimented cells were resuspended in 18 ml cold IMAC buffer A (50 mM Tris/Cl pH 8.0 at 25° C.; 150 mM NaCl; 20 mM imidazole) and were lysed by the addition of 2 ml 10×BugBuster (Merck Millipore). Furthermore, viscosity was reduced with 0.4 Units/ml Benzonase (Merck Millipore). After 20 min incubation at 20° C. and mild agitation, the crude extract was centrifuged (12,000 rcf, 20 min, 4° C.) to sediment cell debris. The clarified supernatant was filtered using a PES membrane with 0.22 μm filter fineness.

Liquid handling during isolation of the target protein with IMAC was performed using an FPLC system (ÄKTA Pure 25, Cytiva Life Science) in combination with a 5 ml Ni-HisTrapHP column (Cytiva Life Science). The filtered supernatant containing the target protein SpA #1 was applied with a flow rate of 5 ml/min onto the IMAC column, previously equilibrated with IMAC buffer A. Unbound proteins were washed out with running buffer until a stable baseline in the UV (280 nm) absorption reading was reached. Bound SpA #1 was eluted stepwise with the elution buffer IMAC B, containing 350 mM imidazole, 50 mM Tris/C¹ and 150 mM NaCl at pH 8.0. Elution fractions containing SpA #1 were collected, supplemented with EDTA (5 mM final) and DTT (20 mM final), and analyzed by SDS-PAGE (Fling und Gregerson 1986). In a final purification step prior to the derivatization with PS1, the IMAC eluate of SpA #1 was subjected to a size-exclusion chromatography on a Superdex 75 Increase 10/300 GL (Cytiva Life Science) column. By this means, monodispersity of the isolated SpA #1 was confirmed and the protein was transferred in the coupling buffer (100 mM Tris/Cl pH 8.5; 150 mM NaCl; 0.5 mM EDTA).

### Example 5: Derivatization of SpA #1 with PS1

Freshly degassed and reduced SpA #1 (304.2 μM, 1.16 μmol, 8.5 mg, 1 eq) in a total volume of 3.8 ml was diluted with the same degassed buffer (100 mM Tris/Cl, 150 mM NaCl, 0.5 mM ETDA, pH 8.5) to a final protein concentration of 20 μM. This solution was stirred under nitrogen atmosphere while 3.5 mg PS1 (3.40 μmol, 3 eq) dissolved in 1.14 ml DMF, which has been illuminated with red light (635 nm) for 5 min, was added in 20 portions over 1 h. The reaction was kept in the dark and stirred for additional 2 h at RT. We found that constant light irradiation was not necessary to maintain the cis-conformation of PS1 during the reaction. Stepwise addition of PS1 helped to prevent the formation of doubly-cross-linked species. The derivatization reaction yielded a high amount of singly-cross-linked species. Further purification by SEC was done to remove only small amounts of cross-linked species and higher oligomers.

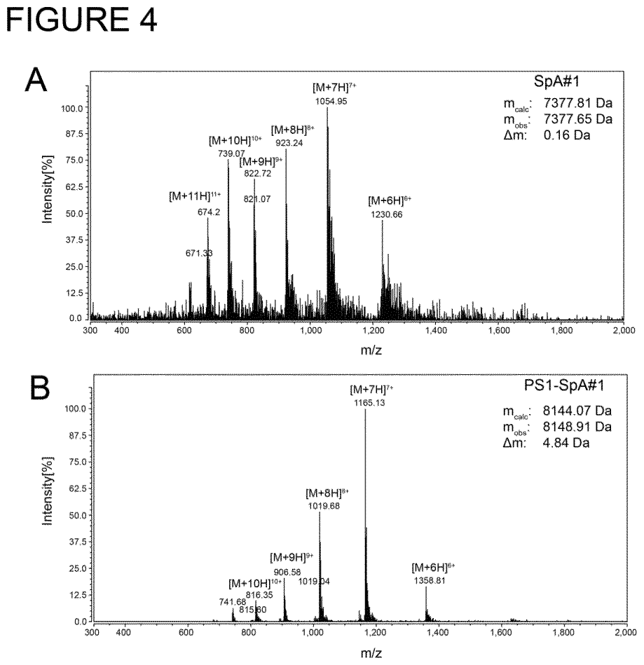

The precise chemical constitution of PS1-SpA #1 was analyzed by electrospray ionization mass spectrometry (ESI-MS) (FIG. 4). ESI-MS analysis was performed with a Thermo Scientific LCQ-Fleet mass spectrometer coupled to a Thermo Scientific Dionex Ultimate 3000 HPLC system. These measurements revealed the correct covalent coupling of PS1 to SpA #1 (FIG. 4B) accompanied by a gain in mass of 767.26 Da.

### Analytical Data:

ESI-MS m/z of SpA #1 (exact mass): calculated: 671.71 [M+11H]¹¹⁺, 738.78 [M+10H]¹⁰⁺, 820.75 [M+9H]⁹⁺, 923.21 [M+8H]⁸⁺, 1054.96 [M+7H]⁷⁺, 1054.96 [M+6H]⁶⁺; found: 671.33, 739.07, 821.07, 923.24, 1054.95, 1230.66.

ESI-MS m/z of PS1-SpA #1 (exact mass): calculated: 741.37 [M+11H]¹¹⁺, 815.40 [M+10H]¹⁰⁺, 905.89 [M+9H]⁹⁺, 1018.99 [M+8H]⁸⁺, 1164.42 [M+7H]⁷⁺, 1358.31 [M+6H]⁶⁺; found: 741.68, 815.60, 906.58, 1019.68, 1165.13, 1358.81.

### Example 6: Affinity Purification of Immunoglobulin G from Cell Culture Supernatant Using a Photoswitchable Affinity Matrix

### Affinity Resin Preparation:

The photoswitchable affinity ligand PS1-SpA #1 was immobilized on a nickel charged IMAC resin (Ni-Sepharose High Performance, Cytiva Life Sciences) via its C-terminal 6×His-tag. The resin was packed into a transparent acryl glass column with 4 mm inner diameter and a packed bed height of 20 mm, corresponding to a settled bed volume (SBV) of 250 μl. An LED array with switchable peak wavelength of 635 nm (red) or 465 nm (blue) was mounted alongside the column housing, surrounded by reflective surfaces, to enable a complete and sufficient illumination of the entire resin material within the column. The assembly was shielded from interfering stray light with an enclosure. At first, the column was equilibrated with 20 CV running buffer (50 mM Tris/Cl pH 8.5, 500 mM NaCl, 40 mM imidazole) at a constant flow rate of 1 ml/min, operated with an FPLC system (ÄKTA Pure 25, Cytiva Life Science). Then, 1.2 mg of purified photoswitchable affinity ligand PS1-SpA #1 was loaded onto the column. The flowthrough was discarded and the column was washed with additional 20 CV of running buffer.

Affinity Purification of Immunoglobulin G from Cell Culture Supernatant:

The chromatography was operated and monitored using an FPLC system (ÄKTA Pure 25, Cytiva Life Science). To follow the course of protein adsorption and desorption the absorbance at 280 nm was recorded (FIG. 5A). A sample of 10 ml cell culture supernatant, containing immunoglobulin G, was dialyzed against running buffer and loaded onto the light switchable affinity matrix (SpA #1-PS1) column at a flow rate of 0.5 ml/min (240 cm/h).

Unbound proteins and impurities (including host cell components) were washed out of the column with 20 CV of running buffer. Sample application and washing steps were conducted under illumination with visible light at 635 nm (red).

Subsequently, elution of bound immunoglobulin G was triggered by illumination of the resin with visible light at 465 nm (blue). By such irradiation, the conformation of the binding protein in the affinity matrix is changed in such a way as to lose binding activity towards the bound IgG, thus effecting instant elution (under constant buffer flow). Regeneration of the resin was done by illumination with visible light at 635 nm (red). All the chromatographic steps after loading were performed at a flow rate of 1 ml/min (480 cm/h) and running buffer as the solely operating fluid. Elution fractions were collected and analyzed in terms of purity and yield using SDS-PAGE, SEC and nanoDSF (FIGS. 5B, C and D).

### Sample Analysis:

SDS-PAGE analysis of the raw material and the elution sample showed characteristic bands for the heavy (approx. 50 kDa) and light (approx. 25 kDa) chain of IgG molecules, indicating a specific binding and subsequent, light-controlled elution of immunoglobulin G. Furthermore, the sample collected during illumination with blue light (465 nm) appeared to be of higher purity compared to the raw material (FIG. 5B). High protein quality of the isolated IgG was also demonstrated by an analytical SEC of the elution sample (FIG. 5C), showing a monodisperse peak corresponding to the IgG molecule (approx. 150 kDa) without any detectable impurities, aggregates or fragments. In addition, the isolated IgG was also analyzed by nano differential scanning fluorimetry (nanoDSF) using a Tycho NT.6 (NanoTemper Technologies, Munich, Germany). The recorded absorbance ratio (350 nm to 330 nm) indicated an immunoglobulin G molecule in its native, folded state with the characteristic thermal unfolding events of the distinct antibody domains (FIG. 5D).

Taken together, the data collected during the isolation of a human IgG1 using a light-controlled affinity matrix, that employs the photoswitchable ligand PS1-SpA #1, shows the general applicability of the invention. Furthermore, the concept of a photoswitch, that can be attached to a protein ligand and modify its binding affinity towards a target in a light dependent manner, appears to be valid. The effect of affinity alteration is most likely a consequence of the distortion of the protein ligand secondary structure upon the photo isomerization of the attached photoswitch PS1 (FIG. 5E).

### Example 7: Synthesis Of 4′-(2-(4-(2-CHIoroacetamido)-3′,5′-dimethoxybiphenyl-3-ylcarbox-amido)acetic acid)-4′-(2-(4-(2-chloroacetamido)-3′,5′-dimethoxybiphenyl-3-ylcarboxamido)acetic acid)-diazene (PS2)

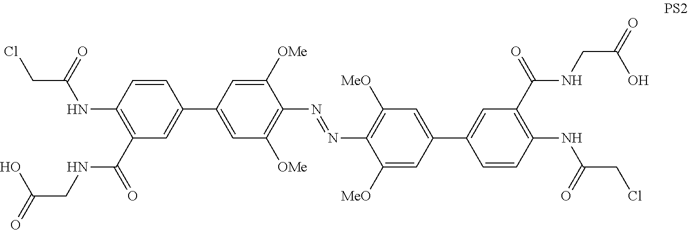 [SMILES: COc1cc(-c2ccc(NC(=O)CCl)c(C(=O)NCC(=O)O)c2)cc(OC)c1/N=N/c1c(OC)cc(-c2ccc(NC(=O)CCl)c(C(=O)NCC(=O)O)c2)cc1OC]

85.0 mg (123.80 μmol, 1 eq) 4′-(2-(4-amino-3′,5′-dimethoxybiphenyl-3-ylcarboxamido)acetic acid)-4′-(2-(4-amino-3′,5′-dimethoxybiphenyl-3-ylcarboxamido)acetic acid)diazene 7 were dissolved in 2.48 mL dry DMF. The solution was stirred and cooled to 0° C. 24.6 μL (309.50 μmol, 2.5 eq) chloroacetyl chloride were added and the reaction was stirred for 1 h at 0° C. Then additional 4.9 μL (0.5 eq) chloroacetyl chloride were added, the reaction was stirred for additional 1 h at 0° C. before 9.8 μL (1 eq) chloroacetyl chloride were added. After stirring for additional 1 h at 0° C. once more 4.9 μL (0.5 eq) chloroacetyl chloride were added and the reaction stirred for additional 7 h at 0° C. 12.5 mL of cold aqueous 0.5 M NaH₂PO₄ (pH 3.5) were added and stirring was continued for 5 min. The dark purple solid was collected by centrifugation and supernatant decantation, washed 3× with H₂O by resuspension centrifugation and decantation and was dried under reduced pressure at 50° C. The product was purified by dissolving the solid in 750 μL DMF at 65° C., 2.25 mL of hot CHCl₃ were added, while stirring was continued for 5 min at 65° C. The mixture was cooled to RT and then kept at 4° C. for 15 h. The dark blue solid was collected by filtration, was washed 2× with 400 μL CHCl₃, dried at atmospheric pressure and afterwards under high vacuum, to yield 90.5 mg (108.78 μmol, 87%).

### Analytical Data:

¹H NMR (500 MHz, DMSO-d6): δ [ppm]=11.97 (s, 2H, NHACCI), 9.35 (t, ³J=5.3 Hz, 2 H, CONHCH₂), 8.59 (d, Jₒᵣₜₕₒ=8.7 Hz, 2 H, C⁵Hₐᵣₒₘ), 8.16 (d, Jₘₑₜₐ=2.2 Hz, 2 H, C²Hₐᵣₒₘ), 8.03 (dd, Jₒᵣₜₕₒ=8.7 Hz, Jₘₑₜₐ=2.2 Hz, 2 H, C⁶Hₐᵣₒₘ), 7.17 (s, 4H, C^{2,6′}Hₐᵣₒₘ), 4.46 (s, 4H, CH₂Cl), 3.99 (d, 3J=5.3 Hz 4 H, CH₂COOH), 3.87 (s, 12H, OCH₃).

¹³C NMR (126 MHz, DMSO-d6): δ [ppm]=171.3 (2×COOH), 168.3 (2×NHCO), 165.2 (2×NHCO), 162.4 (2×C^{3′,5′}ₐᵣₒₘ), 149.8 (2×C^{1′}ₐᵣₒₘ), 140.2 (2×C¹ₐᵣₒₘ), 138.0 (2×C⁴ₐᵣₒₘ), 134.6 (2×C³ₐᵣₒₘ), 132.8 (2×C^{4′}ₐᵣₒₘ), 130.7 (2×C⁶ₐᵣₒₘ), 126.5 (2×C²ₐᵣₒₘ), 120.7 (2×C⁵ₐᵣₒₘ), 104.0 (2×C^{2,6′}ₐᵣₒₘ), 56.6 (4×OCH₃), 43.5 (2×CH₂Cl), 41.5 (2×CH₂COOH).

ESI-MS m/z (exact mass): calculated: 839.18 [M+H]⁺, found: 839.27

### Example 8: Generation Of SpA Variant SpA #2

The Ig-binding protein of the present invention comprises the B domain of protein A (SEQ ID NO. 4), defined as residues 215-268 corresponding to UniProtKB entry P38507. Residues 215-216 in SEQ ID NO. 4 were replaced by Lys213-Ala214-Cys215-Gly216. To provide a strategy for a covalent single point attachment via primary amines, all remaining lysine residues were substituted (Lys218Met, Lys246Arg, Lys260Gln and Lys261Glu). A second cysteine residue was introduced (Ser250Cys), resulting in SpA #2 (SEQ ID NO. 9). The sequence of the Ig-binding affinity ligand SpA #2 was extended at the N-terminus by Met212 and at the C-terminus by Ser269-Ala270-His271-His272-His273-His274-His275-His276.

To generate the SpA #2 variant, suitable for the derivatization with PS1 and PS2, the DNA fragment encoding the SpA #1 variant was mutated via site-directed mutagenesis using appropriate mutagenesis oligo-nucleotides. By this means, amino acids were changed (Lys218Met, Lys246Arg, Lys260Gln and Lys261Glu) with respect to the sequence of SpA #1 (SEQ ID NO. 7) to yield SpA #2.

The mutated DNA fragment encoding SpA #2 was sub-cloned onto the modified expression vector backbone based on pD451sr (ATUM, Newark, CA, USA) by seamless introduction via flanking Sapl DNA restriction sites.

The residues substituted with Cys were intended to provide attachment points for PS1 or PS2 while preserving antibody binding when the photoswitch adopts the cis-conformation (i.e., after illumination at 635 nm) but disturb binding in the trans-conformation (i.e., after illumination at 465 nm). Position Cys215 is located at the N-terminus of the three-helix bundle, position Cys250 is located in the loop between helix 2 and 3.

### Example 9: Expression and Purification of SpA #2

The SpA variant SpA #2 was produced recombinantly as a soluble protein in the cytoplasm of E. coli, isolated with high yields by immobilized metal ion affinity chromatography (IMAC) via the C-terminal 6×His-tag and analyzed by SDS-PAGE according to the experimental procedure described above (analytical data not shown).

### Example 10: Derivatization of SpA #2 with PS2

Freshly degassed and reduced SpA #2 (650 μM, 1.3 μmol, 10 mg, 1 eq) in a total volume of 2 ml was diluted with the same degassed buffer (100 mM sodium borate, 150 mM NaCl, 1 mM TCEP, pH 8.5) to a final protein concentration of 50 μM. This solution was stirred under nitrogen atmosphere while 2.2 mg PS2 (2.6 μmol, 2 eq) dissolved in 0.26 ml DMF, which has been illuminated with red light (635 nm) for 5 min, was added in one portion. The reaction was illuminated with red light and stirred for 16 h at 35° C. It was found that PS2 is stable in the presence of 1 mM TCEP, which prevents the formation of disulfide cross-linked species. The derivatization reaction results in a high amount of intramolecular cross-linked species. Further purification steps by anion exchange chromatography and SEC were performed to remove small amounts of cross-linked species and higher oligomers.

The precise chemical constitution of PS2-SpA #2 was analyzed by electrospray ionization mass spectrometry (ESI-MS). ESI-MS analysis was performed with a Thermo Scientific LCQ-Fleet mass spectrometer coupled to a Thermo Scientific Dionex Ultimate 3000 HPLC system. These measurements verified the successful covalent coupling of PS2 to SpA #2 by a gain in mass of 767.26 Da (analytical data not shown).

### Example 11: Affinity Purification of Immunoglobulin G from Cell Culture Supernatant Using PS2-SpA #2

### Affinity Resin Preparation:

The photoswitchable affinity ligand PS2-SpA #2 was covalently immobilized on N-hydroxysuccinimide (NHS)-activated Sepharose (NHS-Sepharose Fast-Flow, Cytiva Life Sciences) via its primary amines originating from the N-terminus and Lys215.

The resin was packed into a transparent acryl glass column with 4 mm inner diameter and a packed bed height of 20 mm, corresponding to a settled bed volume (SBV) of 250 μl. An LED array with switchable peak wavelength of 635 nm (red) or 465 nm (blue) was mounted alongside the column housing, surrounded by reflective surfaces, to enable a complete and sufficient illumination of the entire resin material within the column. The assembly was shielded from interfering stray light with an enclosure. At first, the column was equilibrated with 20 CV running buffer (50 mM Tris/Cl PH 7.5, 150 mM NaCl) at a constant flow rate of 0.5 ml/min, operated with an FPLC system (ÄKTA Pure 25, Cytiva Life Science). A sample of 5 ml cell culture supernatant, containing immunoglobulin G was loaded onto the device at a flow rate of 0.5 ml/min. Unbound proteins and impurities were washed out of the column with 20 CV of running buffer. Sample application and washing steps were conducted under illumination with visible light at 635 nm (red). Subsequently, elution of bound immunoglobulin G was triggered by illumination of the resin with visible light at 465 nm (blue). Regeneration of the resin was done by illumination with visible light at 635 nm (red). All the chromatographic steps after loading were performed at a flow rate of 0.5 ml/min and running buffer as the solely operating fluid. Elution fractions were collected and analyzed in terms of purity and yield using SDS-PAGE.

### Sample Analysis:

SDS-PAGE analysis of the raw material and the elution sample showed characteristic bands for the heavy (approx. 50 kDa) and light (approx. 25 kDa) chain of IgG molecules, indicating a specific binding and subsequent, light-controlled elution of immunoglobulin G. Protein fractions eluted during illumination with blue light (465 nm) showed the highest protein purity as shown by SDS-PAGE analysis of elution fractions using PS2-SpA #2 as a photoswitchable affinity matrix (data not shown).

In addition, the isolated IgG was also analyzed by an analytical SEC and nano differential scanning fluorimetry (nanoDSF, data not shown) using a Tycho NT.6 (NanoTemper Technologies, Munich, Germany). The results showed that the quality of the protein is on the same level using PS2-SpA #2 instead of PS1-SpA #1 as affinity matrix.

The type of linkage formed between the solid support and the immobilized affinity ligand affects the performance of a photoswitchable affinity matrix in several ways. If the linkage blocks or adversely affects the structure of the immobilized ligand, it will limit the distortion of the protein ligand secondary structure upon the photo isomerization of the attached photoswitch and thus permit binding or elution of the target molecule. A linkage that allows the coupled ligand to leach from the matrix during operation or clean in place procedures will result in contamination of the purified protein and shorten the useful lifetime of the affinity matrix.

These issues were addressed by a photoswitchable affinity ligand according to the third aspect of the invention having no, only one or a defined set of lysine residues. Covalent immobilization of the photoswitchable affinity ligand PS2-SpA #2 on a solid support via amino-reactive linkers (NHS) was possible with a high degree of precision. This further improvement of the light-controlled affinity matrix paves the way for a general application.

### Example 12: 4′-(2-(4-(2,5-dioxo-2,5-dihydro-1H-pyrrol-1-yl)acetamido)-3′,5′-dimethoxybiphenyl-3-ylcarboxamido)acetic acid)-4′-(2-(4-(2,5-dioxo-2,5-dihydro-1H-pyrrol-1-yl)acetamido)-3′,5′-dimethoxybiphenyl-3-ylcarboxamido)acetic acid)diazene. (PS3)

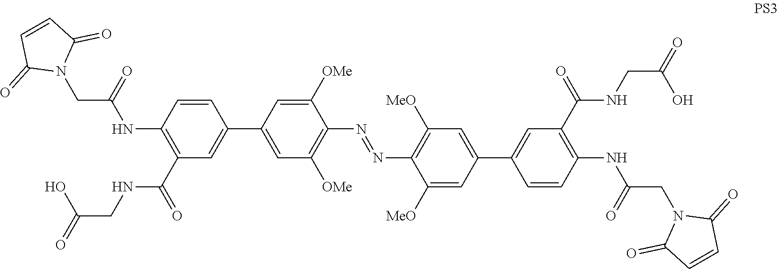 [SMILES: COc1cc(-c2ccc(NC(=O)CN3C(=O)C=CC3=O)c(C(=O)NCC(=O)O)c2)cc(OC)c1/N=N/c1c(OC)cc(-c2ccc(NC(=O)CN3C(=O)C=CC3=O)c(C(=O)NCC(=O)O)c2)cc1OC]

20.0 mg (29.13 μmol, 1 eq) 4′-(2-(4-amino-3′,5′-dimethoxybiphenyl-3-ylcarboxamido)acetic acid)-4′-(2-(4-amino-3′,5′-dimethoxybiphenyl-3-ylcarboxamido)acetic acid)diazene 7 were dissolved in 291 μL dry DMF. The solution was cooled to −20° C., 22.7 mg (131.09 μmol, 4.5 eq) 2,5-Dihydro-2,5-dioxo-1H-pyrrole-1-acetyl chloride (Ilya Nifant'ev et al. (2021) Polymers, 13, 868, doi.org/10.3390/polym13060868) were added while mixing and the reaction was kept at −10 to −20° C. for 2 h. 1.45 mL of cold aqueous 0.5 M NaH₂PO₄ (pH 3.5) were added while mixing. The dark purple solid was collected by centrifugation and supernatant decantation, washed 3× with H₂O by resuspension, centrifugation and decantation and was dried under reduced pressure. Purification was performed by column chromatography (SiO₂, CHCl₃/MeOH containing 2.5% DMF and 2.5% AcOH, step gradient 0, 2, 4, 20% MeOH). The product containing fractions were combined and most of the CHCl₃ and MeOH was removed under reduced pressure at 40° C. The product was precipitated by addition of cold Et₂O (−20° C.) and precipitation was completed for 1 h at −20° C. The solid was collected by centrifugation and supernatant decantation, washed 2× with cold Et₂₀ by resuspension, centrifugation and decantation. The dark red solid was dried at atmospheric pressure and afterwards under high vacuum to yield 15.4 mg (16.02 μmol, 55%).

### Analytical Data:

¹H NMR (500 MHz, DMSO-d6): δ [ppm]=11.96 (s, 2H, NHCO), (bs, 2 H, CONHCH₂), 8.52 (d, Jₒᵣₜₕₒ=8.7 Hz, 2 H, C⁵Hₐᵣₒₘ), 7.93 (d, Jₘₑₜₐ=2.2 Hz, 2 H, C²Hₐᵣₒₘ), 7.91 (dd, Jₒᵣₜₕₒ=8.8 Hz, Jₘₑₜₐ=2.3 Hz, 2 H, C⁶Hₐᵣₒₘ), 7.17 (s, 4H, OCCHCHCO), 7.09 (s, 4H, C^{2,6′}Hₐᵣₒₘ), 4.41 (s, 4H, NHCOCH₂), 3.76 (d, 3J=4.6 Hz 4 H, CH₂COOH), 3.85 (s, 12H, OCH₃).

¹³C NMR (126 MHz, DMSO-d6): δ [ppm]=170.8 (2×COOH), 167.8 (2×NHCO), 165.7 (2×NHCO), 162.4 (2×C^{3′,5′}ₐᵣₒₘ), 152.2 (2×OCCHCHCO), 149.7 (2×C^{1′}ₐᵣₒₘ), 140.3 (2×C¹ₐᵣₒₘ), 135.0 (2×OCCHCHCO), 134.1 (2×C⁴ₐᵣₒₘ), 133.1 (2×C³ₐᵣₒₘ), 132.6 (2×C^{4′}ₐᵣₒₘ), 129.1 (2×C⁶ₐᵣₒₘ), 125.8 (2×C²ₐᵣₒₘ), 120.4 (2× C⁵ₐᵣₒₘ), 103.6 (2×C^{2,6′}ₐᵣₒₘ), 56.4 (4×OCH₃), 43.3 (2×CH₂COOH, 41.1 (2×NHCOCH₂).

ESI-MS m/z (exact mass): calculated: 961.26 [M+H]⁺, found: 961.32

### Example 13: Generation of a Photoswitchable Protein G Variant (Ps2-SpG #1)

The immunoglobulin binding protein described herein is composed of the C1 domain (SEQ ID NO. 12), identified in groups C and G Streptococci. This domain is characterized by an immunoglobulin binding region that exhibits specific affinity for both the Fc and Fab regions of antibodies. The affinity ligand detailed in this instance encompasses a modified C1 domain, which lacks lysine residues—these have been replaced by alternative amino acids, excluding lysine. This modification facilitates targeted covalent attachment via primary amines. Additionally, a substitution of Asn338 with Tyr (Asn338Tyr) enhances stability against alkaline hydrolysis (SEQ ID NO. 15). To further this design, two cysteine residues, Val322Cys and Asp348Cys, were introduced, giving rise to the SpG #1 variant (SEQ ID NO. 18). The SpG #1 variant also features modifications at the N-terminus. The initial Met301 and Ser302 residues are replaced with a short anchor peptide, MATKASK, followed by a polyglycine linker (GGGG), which provides primary amines for solid support coupling. Additionally, the C-terminus was modified with up to eight negatively charged amino acids to aid purification.

The SpG #1 variant was engineered for post-translational modifications with PS2. The introduction of cysteine residues at positions 322 and 348 creates anchoring points for PS2, intended to preserve antibody binding when the photoswitch is in the cis-conformation (post-illumination at 635 nm) and to disrupt binding in the trans-conformation (post-illumination at 465 nm). The cysteine residues are strategically positioned within the loop regions that interconnect beta strand 2 with the alpha helix and between beta strands 3 and 4.

Protein variants were expressed in the cytoplasm of E. coli as soluble fractions and achieved purities exceeding 90% via liquid chromatography. The purified protein was reduced using 20 mM dithiothreitol for a minimum of 1 hour at 25° C. within a pH range of 7.5 to 8.5. Subsequently, excess reducing agents were eliminated via buffer exchange using a HiPrep 26/10 desalting column (Cytiva Life Science). The derivatization reaction of the purified protein was conducted over a period of 12 hours, under conditions of red light illumination. The reaction mixture was composed of 50 UM protein (SpG #1), a twofold molar excess of the photoswitchable azobiaryl compound (PS2), and a tenfold molar excess of tris(2-carboxyethyl) phosphine hydrochloride (TCEP) relative to the protein, deemed as one equivalent. This mixture was prepared in a degassed solution containing 10% (v/v) N,N-dimethylformamide (DMF), 100 mM NaCl, and 50 mM dimethylpiperazine (DMP), with the pH adjusted to 8.5 and the temperature set to 35° C. The purification of the derivatization reaction mixture was conducted through anion exchange chromatography using Capto Q ImpRes resin (Cytiva Life Science) to remove unreacted photoswitchable azobiaryl compound and unreacted protein, as well as small amounts of cross-linked species and higher oligomers. The process utilized 20 mM DMP-buffer at pH 8.5, with gradient elution progressively increasing up to 1 M NaCl. Fractions containing monomeric protein conjugated with a single molecule of PS2 were identified through SDS-PAGE and mass spectrometry analyses, subsequently pooled and concentrated. ESI-MS analysis was performed with a Thermo Scientific LCQ-Fleet mass spectrometer coupled to a Thermo Scientific Dionex Ultimate 3000 HPLC system. These measurements verified the successful covalent coupling of PS2 to SpG #1 by a gain in mass of 767.26 Da (analytical data not shown).

The derivatized and purified protein was then immobilized on NHS-Agarose (NHS-Sepharose Fast-Flow, Cytiva Life Sciences) for a minimum duration of 1 hour at pH 7.0, followed by quenching of excess reactive sites with 1 M ethanolamine at pH 9.5 for at least 1 hour.

### Example 14: Generation of A Photoswitchable Protein L Variant (Ps2-Ppl #1)

The immunoglobulin-binding protein detailed herein comprises Protein L, identified on the bacterial surface of Finegoldia magna. Protein L is notable for its four homologous immunoglobulin-binding domains, as characterized in the Finegoldia magna strain 3316, which exhibit a particular affinity for the light chains of immunoglobulins. This specificity facilitates the purification of a broader range of antibody classes, such as IgA, IgM, IgE, and IgD, which are not amenable to binding by Proteins A or G. Additionally, the light chain specificity of this Ig-binding affinity ligand enables the purification of antibody fragments, including single-chain variable fragments (scFv) and Fab fragments, as well as their fusions. In this context, the affinity ligand incorporates the C2 domain of Protein L, with its lysine residues altered to alternative amino acids, excluding lysine (SEQ ID NO. 17). This modification is strategically implemented to facilitate site-specific immobilization via amino-reactive chemistry, thereby augmenting the ligand's utility for light-controlled affinity purification processes.

Similar to the engineering of SpG #1, the residues preceding Glu327 in the C2 domain of Protein L were replaced with a short anchor peptide, MATKASK, followed by a flexible linker (GGGGAS), which provides primary amines for solid support coupling. Additionally, the C-terminus was modified with up to eight negatively charged amino acids to aid purification. Further, two cysteine residues, Thr344Cys and Asp375Cys, were introduced, giving rise to the PpL #1 variant (SEQ ID NO. 19).

The PpL #1 variant has been specifically designed to undergo post-translational modifications with photoswitchable molecules PS1 and PS2. The strategic insertion of cysteine residues at positions 344 and 375 serves as anchoring points for PS2. This design aims to maintain antibody binding affinity when the photoswitch is in its cis-conformation, which is achieved post-illumination at 635 nm, and to disrupt this binding in the trans-conformation, following illumination at 465 nm. These cysteine residues are strategically positioned on beta strands 2 and 3, effectively spanning the outer strands of a beta-sheet. The beta-sheet in turn interacts with an alpha helix, forming the characteristic fold of the C domains of Protein L. This precise placement ensures the effective modulation of binding characteristics through light-induced structural changes.

The protein was successfully expressed in E. coli and achieved a purity level exceeding 90% following the previously described methodologies. Post-reduction with dithiothreitol (DTT) and subsequent buffer exchange, the protein underwent modification with PS2 as outlined in Example 13. To purify the derivatization reaction mixture, anion exchange chromatography was employed, utilizing Capto Q ImpRes resin (Cytiva Life Science). This step was essential for the removal of unreacted photoswitchable azobiaryl compound, unreacted protein as well as minor cross-linked species and higher oligomeric forms. The procedure adopted 20 mM DMP-Buffer at a pH of 8.5, applying a gradient elution that increased progressively to 1 M NaCl. Fractions that contained the monomeric protein conjugated with a single molecule of PS2 were distinguished via SDS-PAGE and mass spectrometry, then pooled and concentrated for further analysis.

Electrospray ionization mass spectrometry (ESI-MS) analysis was conducted using a Thermo Scientific LCQ-Fleet mass spectrometer in conjunction with a Thermo Scientific Dionex Ultimate 3000 HPLC system. These analyses confirmed the successful covalent attachment of PS2 to the PpL #1 variant, as evidenced by a mass increase of 767.26 Da (analytical data not shown).

Following derivatization and purification, the protein was immobilized on NHS-Agarose (NHS-Sepharose Fast-Flow, Cytiva Life Sciences) for a minimum of 1 hour at pH 7.0. To ensure the deactivation of any remaining reactive sites, a quenching step with 1 M ethanolamine at pH 9.5 was performed for at least 1 hour, preparing the immobilized protein for subsequent applications.

### Example 15: Affinity Purification of Immunoglobulin G from a Complex Polyclonal Immunoglobulin Mixture Using Ps2-Spg #1 and Ps2-Ppl #1

For the functional assessment of the light-switchable affinity matrices, these were encased within a transparent acrylic glass housing, featuring a thickness of 2 mm, to achieve a settled bed volume (SBV) of 250 μl. A LED array equipped with lenses capable of alternating between peak wavelengths of 635 nm (red) and 465 nm (blue) was positioned adjacent to the housing. This configuration ensured thorough and efficient illumination of the entire resin material contained within. The performance of the light-switchable affinity matrix was tested using the polyclonal antibody mixture Cutaquig® (Octapharma) as a substrate.

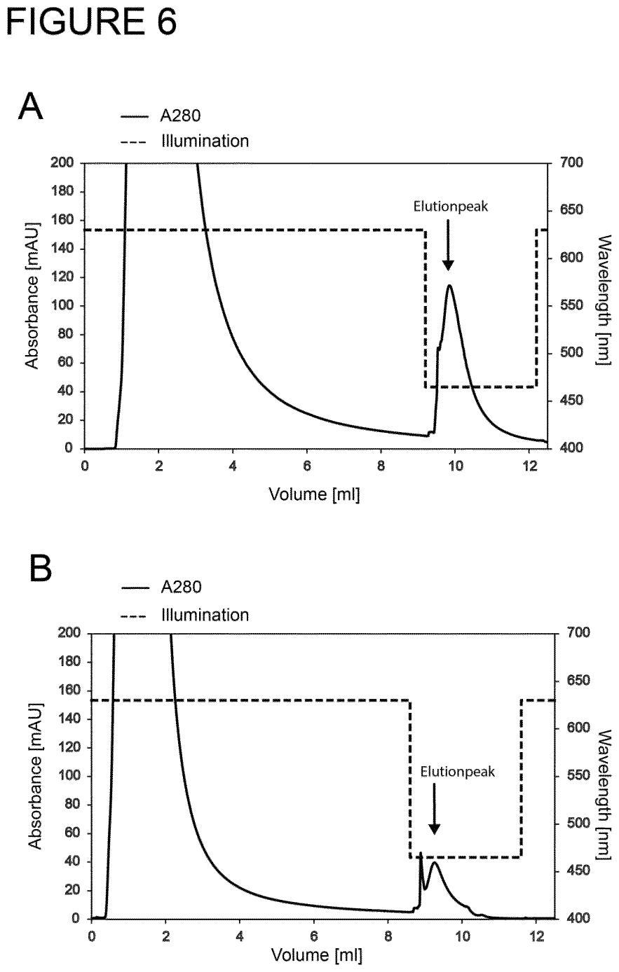

Initially, the column was equilibrated with 3 mL of running buffer (50 mM TrisHCl, pH 7.6, 150 mM NaCl) using an FPLC system (AKTA Pure 25, Cytiva Life Science). The affinity matrix was then loaded with sample to achieve a 60% breakthrough. Unbound proteins and impurities were removed by washing the column with 7.5 mL of the same running buffer. Both, the sample application and washing steps were performed under red visible light illumination at 635 nm to ensure binding conditions. Following this, the elution of bound immunoglobulin G (IgG) was initiated by switching the illumination to blue visible light at 465 nm, which induces the release of the bound IgG. The resin was regenerated by again illuminating with red visible light at 635 nm, preparing it for subsequent use. Except for the sample application, which was executed at 0.2 mL/min, all chromatographic processes steps were conducted at a constant flow rate of 0.5 ml/min, with the running buffer being the only fluid used. The eluted fractions were collected for analysis of purity and yield via SDS-PAGE. The chromatographic profiles obtained from this procedure are depicted in FIG. 6.

The development of light-switchable variants of Protein A, G and L exemplifies the broad applicability of this invention. By employing the azo-biaryl compound (PS2) to modify proteins of varying structures, we have effectively showcased a light-controlled affinity purification method. This accomplishment emphasizes the adaptability of the PS2 modification for binding proteins, facilitating their light-regulated affinity modulation. Such a technique not only improves the efficiency of immunoglobulin G (IgG) purification but also sets the stage for novel purification strategies that exploit light-responsive technologies, marking a significant advancement in the field of biotechnology.

### Example 16: 4′-(2-(4-(2-chloroacetamido)-3′,5′-difluorobiphenyl-3-ylcarboxamido)acetic acid)-4′-(2-(4-(2-chloroacetamido)-3′,5′-difluorobiphenyl-3-ylcarboxamido)acetic acid)diazene (PS4)

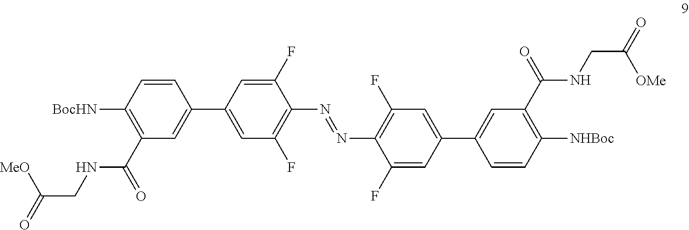 [SMILES: COC(=O)CNC(=O)c1cc(-c2cc(F)c(/N=N/c3c(F)cc(-c4ccc(NC(=O)OC(C)(C)C)c(C(=O)NCC(=O)OC)c4)cc3F)c(F)c2)ccc1NC(=O)OC(C)(C)C]

100 mg (242.70 μmol, 1 eq) 1-(4-bromo-2,6-difluorophenyl)-1-(4-bromo-2,6-difluorophenyl)diazene (S. Okumura et al., J. Org. Chem. 2013, 78, 12090-12105, doi: 10.1021/jo402120w), 221.3 mg (509.67 μmol, 2.1 eq) 3 and 412.2 mg (1.94 mmol, 8 eq) K₃PO₄ were dissolved in 2.4 mL degassed toluene and 1.2 mL degassed H₂O. The mixture was stirred and bubbled with N₂ for 10 min at RT. 17.8 mg (24.27 μmol, 0.1 eq) Pd(dppf)Cl₂ were added and the mixture was stirred and bubbled with N₂ for further 5 min. The reaction vessel was sealed under N₂-atmosphere and placed in an oil bath at 100° C. The reaction was stirred for 2 h 30 min before it was cooled to RT. 20 mL of DCM and 5 mL of H₂O were added and the pH of the aqueous layer was adjusted to 8-9 with 1 M HCl_{aq}, before adding 2 mL of MeOH. Everything was filtered through a thin pad of celite, washed with 20 mL H₂O and the filter cake was extracted with two portions of 25 mL DCM/MeOH (10:1). The filtrate was shaken, the organic layer was separated, dried with MgSO₄, filtered and the solvent was evaporated under reduced pressure yielding 220 mg of crude product, which was purified by column chromatography (SiO₂, DCM/ethyl acetate (12:1)). The solvent of the product containing fractions was evaporated under reduced pressure to yield 104 mg of 9 (119.98 μmol, 49%) as red solid containing the trans-isomer and a minor amount of cis-isomer.

¹H NMR (500 MHz, CDCl₃): Sample contained at the point of measurement app. 20% cis isomer, NMR shifts are reported for the trans isomer, δ [ppm]=10.13 (s, 2H, NHBoc), 8.43 (d, Jₒᵣₜₕₒ=8.8 Hz, 2 H, C⁵Hₐᵣₒₘ), 7.77 (d, Jₘₑₜₐ=2.3 Hz, 2 H, C²Hₐᵣₒₘ), 7.60 (dd, Jₒᵣₜₕₒ=8.9 Hz, Jₘₑₜₐ=2.2 Hz, 2 H, C⁶Hₐᵣₒₘ), 7.28 (m, 2H, CONHCH₂), 7.17 (s, 2H, C2,6′Hₐᵣₒₘ), 7.15 (s, 2H, C^{2,6′}Hₐᵣₒₘ), 4.28 (d, ³J=5.3 Hz, 4 H, CH₂COOMe), 3.86 (s, 6H, COOCH₃), 1.43 (s, 18H, Boc).

¹³C NMR (126 MHz, CDCl₃): δ [ppm]=170.5 (2×COOMe), 169.1 (2×CONHCH₂), 157.1 (1×C3′,5′ₐᵣₒₘ), 155.0 (1×C^{3′,5′}ₐᵣₒₘ), 152.6 (2×NHCOOC(CH₃)₃), 141.5 (2×C1′ₐᵣₒₘ), 131.5 (2×C1ₐᵣₒₘ), 131.2 (2×C4ₐᵣₒₘ), 130.8 (2×C⁶ₐᵣₒₘ), 125.8 (2×C4′ₐᵣₒₘ), 125.3 (2×C2ₐᵣₒₘ), 120.4 (2×C3ₐᵣₒₘ), 120.0 (2×C5ₐᵣₒₘ), 110.60 (1×C2,6′ₐᵣₒₘ), 110.4 (1×C2,6′ₐᵣₒₘ), 80.8 (2×COOC(CH₃)₃), 52.9 (2×COOCH₃), 41.8 (2×CH₂), 28.3 (2×NHCOOC(CH₃)₃).

ESI-MS m/z (exact mass): calculated: 867.30 [M+H]⁺, 889.28 [M+Na]⁺, found: 866.92, 889.15

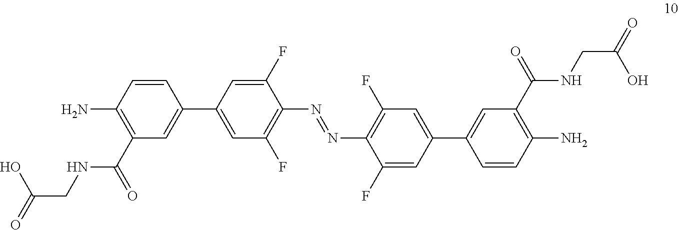 [SMILES: Nc1ccc(-c2cc(F)c(/N=N/c3c(F)cc(-c4ccc(N)c(C(=O)NCC(=O)O)c4)cc3F)c(F)c2)cc1C(=O)NCC(=O)O]

222 μL (3.11 mmol, 60 eq) acetyl chloride were added to a solution of 126 μL (3.11 mmol, 60 eq) dry MeOH in 0.79 mL ethyl acetate at 0° C. The solution was left at 0° C. for 5 min and was then added to 45 mg (51.94 μmol, 1 eq) 9 suspended in 1.04 mL dry DCM at RT. The reaction was stirred at RT for 13 h. The precipitated dianilinium chloride was collected by centrifugation, the supernatant was decanted and the solid was washed twice with DCM by resuspending, centrifugation and decantation. After air drying and drying under reduced pressure the dianilinium chloride was obtained in quantitative yield as a red solid and was directly used in the next step.

The solid was dissolved/suspended in 1.04 mL THF/MeOH (1:1) containing 104 μL (207.76 μmol, 4 eq) 2 M NaOH_{aq} and the mixture was stirred for 5 min, while stirring 936 μL (1.87 mmol, 36 eq) 2 M NaOH_{aq} were added and the reaction was stirred for 3 h at RT. 9 mL H₂O were added, the reaction was stirred for additional 30 min, the pH was adjusted to 3-4 with 1 M HCl_{aq} compared to universal indicator paper and the precipitate was collected by centrifugation. The supernatant was decanted and the solid was washed 3× with 5 mL H₂O each by resuspending, centrifugation and decantation. After drying in vacuum at 40-45° C. 10 was obtained as a red solid in quantitative yield (51.94 μmol) and was directly used in the next step.

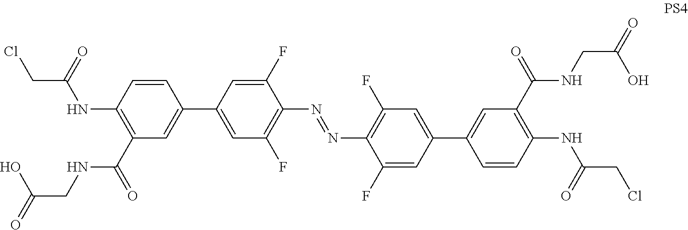 [SMILES: O=C(O)CNC(=O)c1cc(-c2cc(F)c(/N=N/c3c(F)cc(-c4ccc(NC(=O)CCl)c(C(=O)NCC(=O)O)c4)cc3F)c(F)c2)ccc1NC(=O)CCl]

51.94 μmol (1 eq) 10 were dissolved in 1.04 mL dry DMF. The solution was stirred and cooled to 0° C. 20.7 μL (0.26 mmol, 5 eq) chloroacetyl chloride were added and the reaction was stirred for 2.5 h at 0° C. 5 mL of aqueous 0.5 M NaH₂PO₄ (pH 3.5) were added and stirring was continued for 10 min. The red solid was collected by centrifugation and supernatant decantation, washed 3× with 5 mL H₂O by resuspending, centrifugation and decantation and dried under reduced pressure. The product was purified by adding 450 μl DMF to the solid and stirring at 65° C. for 15 min, while stirring 1.35 mL of hot CHCl₃ were added, stirring was continued for 5 min at 65° C. It was cooled to RT and then kept at 4° C. for 6 h. The red solid was collected by filtration, was washed 2× with 500 μL CHCl₃, dried under stream of air and afterwards in high vacuum, to yield 34 mg (42.96 μmol, 83%) of PS4 as red solid.

¹H NMR (500 MHz, DMSO-d6): Compound was characterized as its double diisopropylethyl ammonium salt, multiplet at 2.52-2.48 of DIEA coincides with solvent signal, δ [ppm]=12.28 (s, 2H, NHCO), 9.11 (bs, 2 H, CONHCH₂), 8.59 (d, Jₒᵣₜₕₒ=8.8 Hz, 2 H, C5Hₐᵣₒₘ), 8.15 (d, Jₘₑₜₐ=2.3 Hz, 2 H, C2Hₐᵣₒₘ), 8.02 (dd, Jₒᵣₜₕₒ=8.8 Hz, Jₘₑₜₐ=2.2 Hz, 2 H, C6Hₐᵣₒₘ), 7.86 (s, 2H, C2,6′Hₐᵣₒₘ), 7.83 (s, 2H, C2,6′Hₐᵣₒₘ), 4.45 (s, 4H, NHCOCH₂), 3.83 (bs, CH₂COO), 2.89-2.73 (m, 4H, DIEA), 2.52-2.48 (m, 4H, DIEA), 1.27-1.00 (m, 30H, DIEA).

¹³C NMR (126 MHz, DMSO-d6): Signal of DIEA at app. 40.0 ppm coincides with solvent signal, δ [ppm]=171.6 (2×COO), 167.4 (2×CONHCH₂), 165.6 (2×NHCO), 156.5 (1×C3′,5′ₐᵣₒₘ), 154.5 (1×C3′,5′ₐᵣₒₘ), 138.9 (2×C1′ₐᵣₒₘ), 133.3 (2×C4ₐᵣₒₘ), 130.9 (2×C1ₐᵣₒₘ), 129.9 (2×C6ₐᵣₒₘ), 128.0 (2×C4′ₐᵣₒₘ), 126.4 (2×C2ₐᵣₒₘ), 124.6 (2×C3ₐᵣₒₘ), 120.6 (2×C5ₐᵣₒₘ), 111.3 (1×C2,6′ₐᵣₒₘ), 110.5 (1×C2,6′ₐᵣₒₘ), 51.3 (4× C, DIEA), 43.6 (2×CH₂Cl), 42.7 (2×CH₂COO—), 18.6 (8×C, DIEA), 13.8 (2×C, DIEA).

ESI-MS m/z (exact mass): calculated: 789.09 [M−H]; 394.04 [M−2H]2-, found: 789.26, 394.38

### Example 17: Light Induced Isomerization of 4′-(2-(4-(2-Chloroacetamido)-3′,5′-Difluorobiphenyl-3-ylcarboxamido)acetic acid)-4′-(2-(4-(2-chloroacetamido)-3′,5′-difluorobiphenyl-3-ylcarboxamido)acetic acid)diazene (PS4)

A 1 mM solution of PS4 in DMF was prepared with exclusion of light and 5 μL of this solution were subjected to HPLC analysis as described in example 2 with an eluent B gradient of 5 to 50%. This solution was then illuminated with yellow light (LED-593 nm, 350 mA), blue light (LED-450 nm, 350 mA) and UV light (312 nm, INTAS UV Transilluminator) for 2 min each and 5 μL of each illumination step were subjected to HPLC analysis in the same manner. Analysis showed good light induced switching between the trans- and cis-state with following trans/cis ratios: dark (92:8), yellow (14:86), blue (67:33), UV (80:20).

To determine t_{1/2} (cis→trans) at 25° C. the solution was again illuminated with yellow light (LED-593 nm, 350 mA) for 2 min, kept in the dark and 5 μL each were analyzed by HPLC in the same manner at 0, 20, 40, . . . , 120 min. t_{1/2} (cis→trans) was determined to be ≥1 d at 25° C.

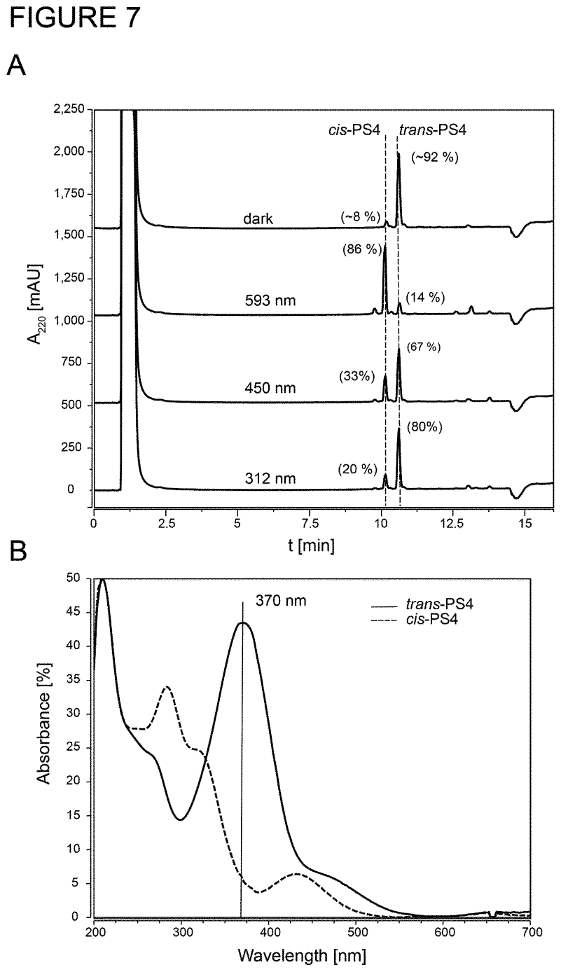

UV-Vis spectra of the cis- and trans-isomers were also recorded with the Thermo Scientific Dionex Ultimate 3000 HPLC system with Diode Array Detector (FIG. 7).

1. Photoswitchable azobiaryl compound according to formula (I) 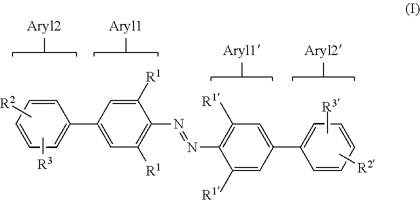
wherein:
R¹ and R^{1′} are independently selected from the group comprising H, F, Cl, Br, (CH₂),CH₃, CH((CH₂)ₙCH₃)((CH₂)ₓCH₃),C((CH₂),CH₃)((CH₂)ₓCH₃)((CH₂) 2CH₃), (CH₂),CH((CH₂)ₓCH₃)—((CH₂)_{z}CH₃), (CH₂),C((CH₂)ₓCH₃)((CH₂)_{y}CH₃)((CH₂)_{z}CH₃), O(CH₂)ₙCH₃, OCH((CH₂)ₙCH₃)((CH₂)ₓCH₃), OC((CH₂),CH₃)—((CH₂)ₓCH₃)((CH₂)_{z}CH₃), O(CH₂),CH((CH₂)ₓCH₃)((CH₂)_{z}CH₃),O(CH₂),C((CH₂)ₓCH₃)((CH₂)_{y}CH₃)((CH₂) 2CH₃), S(CH₂),CH₃, SCH((CH₂)ₙCH₃)((CH₂)ₓCH₃), SC((CH₂) CH₃)((CH₂)ₓCH₃)((CH₂) 2CH₃), S(CH₂),CH((CH₂)ₓCH₃)((CH₂)_{z}CH₃), S(CH₂) DC((CH₂)ₓCH₃)((CH₂)_{y}CH₃)((CH₂)_{z}CH₃), NH₂, NH(CH₂),CH₃, NHCH((CH₂)ₙCH₃)—((CH₂)ₓCH₃), NHC((CH₂)ₙ CH₃)((CH₂)ₓCH₃)((CH₂)_{z}CH₃), NH(CH₂),CH((CH₂)ₓCH₃)((CH₂)_{z}CH₃), NH(CH₂) DC—((CH₂)ₓCH₃)—((CH₂)_{y}CH₃)((CH₂)_{z}CH₃), N((CH₂),CH₃)₂, N(CH((CH₂)ₙCH₃)((CH₂)ₓCH₃))₂, N(C((CH₂)ₙ,CH₃)—((CH₂)ₓCH₃)((CH₂)_{z}CH₃))₂, N((CH₂)ₙCH((CH₂)ₓCH₃)((CH₂)_{z}CH₃))₂, N((CH₂)ₙC((CH₂)ₓCH₃)((CH₂)_{y}CH₃)—((CH₂)_{z}CH₃))₂, aziridin-1-yl, azetidin-1-yl, pyrrolidin-1-yl, piperidin-1-yl, azepan-1-yl, azocan-1yl, morpholin-1-yl, 4-methyl-piperazin-1-yl,
wherein n may be any integer from 0 to 10,
wherein x may be any integer from 0 to 10,
wherein y may be any integer from 0 to 10, and
wherein z may be any integer from 0 to 10;
wherein at least one of R¹ or R^{1′} is not H;
R² and R^{2′} are independently selected from the group comprising SO₃H, SO₃Li, SO₃Na, SO₃K, COOH, COONHS, CONH₂, CONH—(CH₂)ₙCH₃, CONH—((CH₂)ₙCH₃)((CH₂)_{z}CH₃), CONH—(CH₂)ₙSO₃H, CONH—(CH₂)ₙSO₃Li, CONH—(CH₂)ₙSO₃Na, CONH—(CH₂)ₙSO₃K, CONH—(CH₂)ₙCCH, CONH—(CH₂)ₙN₃, CONH—(CH₂)ₙNH₂, CONH—(CH₂)ₙCOOH, CONH—(CH₂)ₙNHAcI, CONH—(CH₂)ₙNHAcBr, CONH—(CH₂)ₙNHAcCl, CONH—(CH₂)ₙ N(maleimide), CONH—(CH₂)ₙ—NH(2-chloromethyl acrylate), CONH—(CH₂)ₙ—NH(vinylsulfonate), CONH—(CH₂)ₙ—NHCOPhF₅, CONH—(CH₂)ₙ—NHSO₂PhF₅, CONH—(CH₂)ₙCOONHS, CONH-PEG-OH, CONH-PEG-NH₂, CONH-PEG-COOH, CONH-PEG-COONHS, CONH-PEG-O(CH₂)ₙSO₃H, CONH-PEG-O(CH₂)ₙSO₃Li, CONH-PEG-O(CH₂)ₙSO₃Na, CONH-PEG-O(CH₂)ₙSO₃K, CONH—PEG-NHAcI, CONH-PEG-NHAcBr, CONH-PEG-NHAcCl, CONH-PEG-N(maleimide), CONH-PEG-NH(2-chloromethyl acrylate), CONH-PEG-NH(vinylsulfonate), CONH-PEG-NHCOPhF₅, CONH-PEG-NHSO₂PhF₅, CONH-PEG-N₃, CONH-PEG-OCH₂CCH, CONH-PEG-NHCH₂CCH, CONH-PEG-N(CH₂CCH)₂, CONH-PEG-NHCO(CH₂)ₙCOOH, CONH-PEG-NHCO(CH₂)ₙCOONHS, CONH—(Xaa)ₙ-OH, CONH—(Xaa)ₙ-NH(CH₂)ₙSO₃H, CONH—(Xaa)ₙ-NH(CH₂) SO₃Li, CONH—(Xaa),—NH(CH₂)ₙSO₃Na, CONH—(Xaa)ₙ-NH(CH₂)ₙSO₃K, CONH—(Xaa)ₙ-OMe, CONH—(Xaa)ₙ-ONHS, CONH—(Xaa)ₙ-ONHS, CONH—(Xaa)ₙ-NH(CH₂)ₙCCH, CONH—(Xaa)ₙ-NH(CH₂)ₙN₃, CONH—(Xaa)ₙ-NH(CH₂)_{z}NHAcI, CONH—(Xaa)ₙ-NH(CH₂)_{z}NHAcBr, CONH—(Xaa)ₙ-NH(CH₂)_{z}NHAcCl, CONH—(Xaa)ₙ-NH(CH₂)_{z}—N(maleimide), CONH—(Xaa)ₙ-NH(CH₂)_{z}NH(2-chloromethyl acrylate), CONH—(Xaa)ₙ-NH(CH₂)_{z}NH(vinylsulfonate), CONH—(Xaa)ₙ-NH(CH₂)_{z}NHCOPhF₅, CONH—(Xaa)ₙ-NH(CH₂)_{z}NHSO₂PhF₅, CONH—(Xaa)ₙ-N₃, CONH—(Xaa)ₙ-OCH₂CCH, CONH—(Xaa)ₙ-NHCH₂CCH, CONH—(Xaa)ₙ-N(OCH₂CCH)₂, CONH—(Xaa)ₙ-NH—(CH₂)ₙCOOH, CONH—(Xaa)ₙ-NH—(CH₂)ₙCOONHS, CONH—(Xaa)-NH-PEG-OH, CONH—(Xaa)ₙ-NH-PEG-NH₂, CONH—(Xaa)ₙ-NH-PEG-COOH, CONH—(Xaa)ₙ-NH-PEG-COONHS, CONH—(Xaa)ₙ-NH-PEG-NHAcI, CONH—(Xaa)ₙ-NH-PEG-NHAcBr, CONH—(Xaa)ₙ-NH-PEG-NHAcCl, CONH—(Xaa)ₙ-NH-PEG-N(maleimide), CONH—(Xaa)ₙNH-PEG-NH(2-chloromethyl acrylate), CONH—(Xaa)ₙ-NH-PEG-NH(vinylsulfonate), CONH—(Xaa)ₙ-NH-PEG-NHCOPhF₅, CONH—(Xaa)ₙ-NH-PEG-NHSO₂PhF₅, CONH—(Xaa)ₙ-NH-PEG-N₃, CONH—(Xaa)ₙ-NH-PEG-OCH₂CCH, CONH—(Xaa)ₙ-NH-PEG-NHCH₂CCH, CONH—(Xaa)ₙ-NH-PEG-N(CH₂CCH)₂, CONH—(Xaa)ₙ-NH-PEG-NHCO(CH₂)ₙCOOH, CONH—(Xaa)ₙ-NH-PEG-NHCO(CH₂)ₙCOONHS,
wherein Xaa can be any canonical or non-canonical amino acid,
wherein n may be any integer from 0 to 10, and
wherein z may be any integer from 0 to 10;
R³ and R^{3′} are independently selected from the group comprising H, NH₂, NHCO(C1-C6-alkyl), NHCO(C1-C6-haloalkyl),NHBoc, NHCbz, NHalloc, NH(CH₂)ₙCH₃, NHCH((CH₂) CH₃)((CH₂)ₓCH₃), NHC((CH₂)ₙ CH₃)((CH₂)ₓCH₃)((CH₂)_{z}CH₃), NH(CH₂)ₙCH((CH₂)ₓCH₃)((CH₂)_{z}CH₃), NH(CH₂)ₙC((CH₂)ₓCH₃)((CH₂)_{y}CH₃)((CH₂) 2CH₃), N((CH₂)ₙ CH₃)₂, N(CH((CH₂)ₙCH₃)—((CH₂)ₓCH₃))₂, N(C((CH₂)ₙCH₃)((CH₂)ₓCH₃)((CH₂) 2CH₃))₂, N((CH₂) CH((CH₂)ₓCH₃)—((CH₂)_{z}CH₃))₂, N((CH₂)ₙC((CH₂)ₓCH₃)((CH₂)_{y}CH₃)((CH₂)_{z}CH₃))₂, aziridin-1-yl, azetidin-1-yl, pyrrolidin-1-yl, piperidin-1-yl, azepan-1-yl, azocan-1yl, morpholin-1-yl, 4-methyl-piperazin-1-yl, NHCH₂CHCH₂, N(CH₂CHCH₂)₂, NHCH₂CCH, N(CH₂CCH)₂, NHCOCCH, NHCO(CH₂)ₙN₃, NHacrylate, NH(2-chloromethyl acrylate), NH(vinyl sulfonate), N(maleimide), N(2-bromomaleimide), N(2,3-dibromomaleimide), NHCOaryl, NHCOhaloaryl, NHSO₂aryl, NHSO₂haloaryl, (CH₂)ₙN(maleimide), (CH₂)ₙN(2-bromomaleimide), (CH₂)ₙN(2,3-dibromomaleimide), NHCO(CH₂)ₙN(maleimide), NHCO(CH₂)ₙN(2-bromomaleimide), NHCO(CH₂)ₙN(2,3-dibromomaleimide), NCS, NCO, NH(CH₂)ₙCH(O)CH₂, N((CH₂)ₙCH(O)CH₂)₂, NAcCH₂CH(O)CH₂,
wherein n may be any integer from 0 to 10, and wherein z may be any integer from 0 to 10.

2. Photoswitchable azobiaryl compound according to claim 1, wherein Aryl¹-Aryl² and Aryl^{1′}-Aryl^{2′} are multifunctionalized biaryl moieties, preferably wherein at least one R¹ and at least one R^{1′} is not hydrogen, more preferably wherein both R¹ and both R^{1′} are not hydrogen,
and/or wherein at least one, preferably both, of R² and R^{2′} is CONH-Xaa-OH (Xaa=any canonical or non-canonical amino acid), or wherein at least one, preferably both, of R² and R^{2′} comprise a linker selected from the group consisting of peptides, bifunctional alkanes, poly(alkylene oxides), preferably wherein said poly(alkylene oxides) has a molecular weight selected from the group consisting of between about 100 g/mol and about 80,000 g/mol, more preferably between about 100 g/mol and 6,000 g/mol,
and/or wherein at least one, preferably both, of R³ and R^{3′} is a substituent selected from the group comprising amines, acrylamides, NHCO(C₁-C₆-haloalkyl), vinyl sulfonates, isothiocyanates, isocyanates, epoxides, maleimides, haloaryl carboxy- and haloaryl sulfonamides as defined in claim 1.

3. Photoswitchable azobiaryl compound according to claim 1, wherein the compound is 4′-(2-(4-(2-iodoacetamido)-3′,5′-dimethoxybiphenyl-3-ylcarboxyamido)acetic acid)-4′-(2-(4-(2-iodoacetamido)-3′,5′-dimethoxybiphenyl-3-ylcarboxyamido)acetic acid)diazene.

4. Photoswitchable azobiaryl compound according claim 1, wherein the compound is 4′-(2-(4-(2-chloroacetamido)-3′,5′-dimethoxybiphenyl-3-ylcarboxyamido)acetic acid)-4′-(2-(4-(2-chloroacetamido)-3′,5′-dimethoxybiphenyl-3-ylcarboxyamido)acetic acid)diazene.

5. Photoswitchable azobiaryl compound according to claim 1, wherein the compound is 4′-(2-(4-(2,5-dioxo-2,5-dihydro-1H-pyrrol-1-yl)acetamido)-3′,5′-dimethoxybiphenyl-3-ylcarboxamido)acetic acid)-4′-(2-(4-(2,5-dioxo-2,5-dihydro-1H-pyrrol-1-yl)acetamido)-3′,5′-dimethoxybiphenyl-3-ylcarboxamido)acetic acid)diazene.

6. Photoswitchable azobiaryl compound according to claim 1, wherein the compound is 4′-(2-(4-(2-iodoacetamido)-3′,5′-difluorobiphenyl-3-ylcarboxamido)acetic acid)-4′-(2-(4-(2-iodoacetamido)-3′,5′-difluorobiphenyl-3-ylcarboxamido)acetic acid)diazene, or wherein the compound is 4′-(2-(4-(2-chloroacetamido)-3′,5′-difluorobiphenyl-3-ylcarboxyamido)acetic acid)-4′-(2-(4-(2-chloroacetamido)-3′,5′-difluorobiphenyl-3-ylcarboxyamido)acetic acid)diazene,
or wherein the compound is 4′-(2-(4-(2,5-dioxo-2,5-dihydro-1H-pyrrol-1-yl)acetamido)-3′,5′-difluorobiphenyl-3-ylcarboxamido)acetic acid)-4′-(2-(4-(2,5-dioxo-2,5-dihydro-1H-pyrrol-1-yl)acetamido)-3′,5′-difluorobiphenyl-3-ylcarboxamido)acetic acid)diazene, or wherein the compound is 4′-(2-(4-(2-iodoacetamido)-3′,5′-dichlorobiphenyl-3-ylcarboxamido)acetic acid)-4′-(2-(4-(2-iodoacetamido)-3′,5′-dichlorobiphenyl-3-ylcarboxamido)acetic acid)diazene,
or wherein the compound is 4′-(2-(4-(2-chloroacetamido)-3′,5′-dichlorobiphenyl-3-ylcarboxyamido)acetic acid)-4′-(2-(4-(2-chloroacetamido)-3′,5′-dichlorobiphenyl-3-ylcarboxyamido)acetic acid)diazene,
or wherein the compound is 4′-(2-(4-(2,5-dioxo-2,5-dihydro-1H-pyrrol-1-yl)acetamido)-3′,5′-dichlorobiphenyl-3-ylcarboxamido)acetic acid)-4′-(2-(4-(2,5-dioxo-2,5-dihydro-1H-pyrrol-1-yl)acetamido)-3′,5′-dichlorobiphenyl-3-ylcarboxamido)acetic acid)diazene.

7. Photoswitchable affinity ligand comprising an affinity ligand in stable association with the photoswitchable azobiaryl compound according to claim 1.

8. Photoswitchable affinity ligand according to claim 7, wherein the photoswitchable azobiaryl compound is stably associated with two conjugation sites within the affinity ligand in a bi- or difunctional manner.

9. Photoswitchable affinity ligand according to claim 7, wherein the affinity ligand is selected from the group comprising immunoglobulin (Ig)-binding proteins, preferably selected from the group comprising protein A, protein G and protein L or variants thereof with the ability to specifically bind to immunoglobulins.

10. Photoswitchable affinity ligand according to claim 7, wherein the affinity ligand comprises the B domain of protein A (SEQ ID NO. 4) optionally substituted with up to two cysteine residues, or comprises a protein domain having at least 80% sequence identity to SEQ ID NO. 4, preferably wherein the affinity ligand comprises a protein domain having the amino acid sequence of SEQ ID NO. 6 or SEQ ID NO. 7, more preferably wherein the affinity ligand comprises a protein domain having the amino acid sequence of SEQ ID NO. 7,
or wherein the affinity ligand comprises the lysine-deficient B domain of protein A (SEQ ID NO. 5) optionally substituted with up to two cysteine residues, or comprises a protein domain having at least 80% sequence identity to SEQ ID NO. 5, preferably wherein the affinity ligand comprises a protein domain having the amino acid sequence of SEQ ID NO. 8, SEQ ID NO. 9, SEQ ID NO. 10 or SEQ ID NO. 11, more preferably wherein the affinity ligand comprises a protein domain having the amino acid sequence of SEQ ID NO. 11,
or wherein the affinity ligand comprises at least one of the three homologous domains of protein G, defined as C1 (SEQ ID NO. 12), C2 (SEQ ID NO. 13) and C3 (SEQ ID NO. 14) optionally carrying up to two substitutions of wild-type residues with cysteines or comprises a protein domain having at least 80% sequence identity to SEQ ID NO. 12, SEQ ID NO. 13 or SEQ ID NO. 14, or wherein the affinity ligand comprises a lysine-deficient domain of protein G, defined as C1 (SEQ ID NO. 15) optionally carrying up to two substitutions of wild-type residues with cysteines or comprises a protein domain having at least 80% sequence identity to SEQ ID NO. 15,
or wherein the affinity ligand comprises a C-domain of protein L optionally carrying two substitutions of wild-type residues with up to two cysteines or a protein domain having at least 80% sequence identity thereto, preferably wherein the C-domain of protein L is the C2 (SEQ ID NO. 16) or a protein domain having at least 80% sequence identity thereto,
or wherein the affinity ligand comprises a lysine-deficient C-domain of protein L optionally carrying two substitutions of wild-type residues with up to two cysteines or a protein domain having at least 80% sequence identity thereto, preferably wherein the C-domain of protein L is C2 (SEQ ID NO. 17) or a protein domain having at least 80% sequence identity thereto.

11. Photoswitchable affinity matrix comprising:
1) a solid support, and
2) a) a photoswitchable azobiaryl compound according to claim 1 in stable association with an affinity ligand, wherein the photoswitchable azobiaryl compound and/or the affinity ligand are in stable association with the solid support,
or
b) a photoswitchable affinity ligand in stable association with the solid support.

12. Use of a photoswitchable compound for isolating and/or purifying a target molecule.

13. Use of a photoswitchable compound for isolating and/or purifying a target molecule, wherein the photoswitchable compound is a photoswitchable azobiaryl compound comprising at least two substituted biphenyl moieties, preferably wherein the photoswitchable compound is a photoswitchable azobiaryl compound according to claim 1.

14. Use of a photoswitchable affinity ligand comprising an affinity ligand in stable association with a photoswitchable compound for isolating and/or purifying a target molecule.

15. Use of a photoswitchable affinity ligand comprising an affinity ligand in stable association with a photoswitchable compound for isolating and/or purifying a target molecule, wherein the photoswitchable affinity ligand comprises an affinity ligand in stable association with a photoswitchable azobiaryl compound comprising at least two substituted biphenyl moieties, preferably wherein the photoswitchable affinity ligand is a photoswitchable affinity ligand according to claim 7.

16. Use of a photoswitchable affinity matrix according to claim 11 for isolating and/or purifying a target molecule.

17. Method of isolating and/or purifying a target molecule, wherein the method comprises the steps of:
a) providing a composition comprising a target molecule,
b) contacting the composition with an affinity matrix comprising:
a photoswitchable compound in stable association with an affinity ligand and a solid support to form an affinity matrix,
for a time sufficient to allow specific binding of the target molecule to the affinity matrix,
c) washing the affinity matrix with a washing solution to remove the components of the composition not specifically bound to the affinity matrix,
d) irradiating the affinity matrix with (a) wavelength(s) of light of at least about 400 nm in order to cause a loss of specific binding of the affinity matrix to the target molecule, and
e) eluting the target molecule from the affinity matrix with an eluent.

18. Method of isolating and/or purifying a target molecule, wherein the method comprises the steps of:
a) providing a composition comprising a target molecule,
b) contacting the composition with an affinity matrix comprising:
i) a photoswitchable azobiaryl compound comprising at least two substituted biphenyl moieties, preferably the photoswitchable azobiaryl compound according to claim 1 in stable association with an affinity ligand, wherein the photoswitchable azobiaryl compound and the affinity ligand are in stable association with a solid support to form an affinity matrix,
or
ii) a photoswitchable affinity ligand in stable association with a solid support to form an affinity matrix,
or
iii) a photoswitchable affinity matrix,
for a time sufficient to allow specific binding of the target molecule to the affinity matrix,
c) washing the affinity matrix with a washing solution to remove the components of the composition not specifically bound to the affinity matrix,
d) irradiating the affinity matrix with (a) particular wavelength(s) of light in order to cause a loss of specific binding of the affinity matrix to the target molecule, and
e) eluting the target molecule from the affinity matrix with an eluent.
